(19) United States
(12) Patent Application Publication
(10) Pub. No.: US 2025/0164474 A1
(43) Pub. Date: May 22, 2025

(54) METHODS AND RELATED ASPECTS FOR PERFORMING LABEL-FREE SINGLE-MOLECULE IMMUNOASSAYS

(71) Applicant: ARIZONA BOARD OF REGENTS ON BEHALF OF ARIZONA STATE UNIVERSITY, Scottsdale, AZ (US)

(72) Inventors: Shaopeng WANG, Chandler, AZ (US); Xiaoyan ZHOU, Tempe, AZ (US); Shuang Zhou, Tempe, AZ (US)

(73) Assignee: ARIZONA BOARD OF REGENTS ON BEHALF OF ARIZONA STATE UNIVERSITY, Scottsdale, AZ (US)

(21) Appl. No.: 18/956,466

(22) Filed: Nov. 22, 2024

Related U.S. Application Data
(60) Provisional application No. 63/602,087, filed on Nov. 22, 2023.

(51) Int. Cl. G01N 33/543; B01L 3/00; G01N 21/17; G01N 33/68

(52) U.S. Cl. CPC G01N 33/54366; B01L 3/502761; G01N 21/17; G01N 33/6869; B01L 2300/0663; B01L 2300/087; G01N 2021/1765; G01N 2333/5412; G01N 2333/58; G01N 2800/52

(57) ABSTRACT

Provided herein are methods of detecting target molecules. The methods include contacting a sample comprising the target molecule with a substrate that comprises a plurality of capture antibodies, or antigen binding portions thereof, that specifically bind to the target molecule to form captured target molecules, and contacting the captured target molecules with a plurality of detection antibodies, or antigen binding portions thereof, that bind to the captured target molecules to form target molecule complexes. The methods also include taking images of the target molecule complexes to produce imaged target molecule complexes, and quantifying an amount of target molecules in the sample using the imaged target molecule complexes. Additional methods as well as related devices and systems are also provided.

### CROSS-REFERENCE TO RELATED APPLICATIONS

This application claims priority to, and the benefit of, U.S. Provisional Patent Application Ser. No. 63/602,087, filed Nov. 22, 2023, the disclosure of which is incorporated herein by reference.

### STATEMENT REGARDING FEDERALLY SPONSORED RESEARCH OR DEVELOPMENT

This invention was made with government support under R01GM140193 awarded by the National Institutes of Health. The government has certain rights in the invention.

### BACKGROUND

Detection and quantification of biomarkers at extremely low levels is important to disease detection, monitoring and treatment. While numerous approaches have been developed, the enzyme-linked immunosorbent assay remains “gold standard” method for the detection of protein biomarkers in both clinical use and basic research. However, its dynamic range (log 3), detection limit (picomolar range) and assay time (3-8 h) fall short of the requirement of early disease diagnosis in clinical settings. Recently, the development of single-molecule imaging has made it possible to visualize individual binding complexes, leading to greatly improved sensitivity, reliability and more depth of information compared to conventional methods. Newer methods—including single-molecule enzyme-linked immunosorbent assays (SiMoAs), single-molecule recognition through equilibrium Poisson sampling (SIMREPS), single-molecule augmented capture (SMAC) and time-resolved digital immunoassay (TD-immunoassay)—have been developed to improve detection limit. However, to enable single molecule detection, all of these methods need to amplify the original binding signal, by means of enzymatic amplification, fluorescence or nanoparticle labeling. These signal-amplification techniques suffer from inherent limitations. Methods based on enzyme amplification and gold nanoparticle labeling are limited by matrix effects and unwanted background signal arising from nonspecific reagent binding to assay substrate. Fluorescence-based methods are limited by photobleaching and ubiquitous autofluorescence of sample matrix components. Such limitations make these single-molecule assays incapable of measuring biomarkers directly in undiluted complex sample matrices, especially in whole blood. Moreover, these assays predominantly rely on end-point detection and fail to fully exploit real-time kinetic data. As most interactions occurring on the sensor surface follow first-order binding kinetics, and replicate measurements at the same time point follow a Poisson distribution, real-time data at each time point can contribute meaningfully to the final outcome. Therefore, fully leveraging real-time kinetic information can significantly enhance the precision and reliability of the final results.

Accordingly, there is a need for additional methods for the detection and quantification of low-abundance blood biomarkers.

### SUMMARY

In some aspects, the present disclosure provides a single-molecule immunoassay is a reliable technique for the detection and quantification of low-abundance blood biomarkers, which are important for early disease diagnosis and biomedical research. However, current single-molecule methods predominantly rely on end-point-detection and necessitate signal amplification via labelling, which brings a variety of unwanted effects, like matrix effect and autofluorescence interference. In some aspects, the present disclosure provides a real-time mass imaging-based label-free single-molecule immunoassay (LFSMiA). Featuring plasmonic scattering microscopy-based mass imaging, a 2-step sandwich assay format-enabled background reduction, minimization of matrix effect by dynamic tracking of single binding events, and fully leveraging real-time data for improved measurement precision through a Gaussian Bayes algorithm, the LFSMiA enables ultra-sensitive and direct protein detection at single-molecule level in neat blood sample matrices. We demonstrated that the LFSMiA can measure interleukin-6 (IL-6) and prostate-specific antigen (PSA) in buffer, undiluted serum and whole blood with sub-femtomolar limit of detection and eight log of dynamic ranges. Moreover, comparable performance was achieved with an inexpensive miniaturized setup. To show its translational potential to clinical settings and point-of-care diagnostics, we examined the detection of N-terminal pro b-type natriuretic peptide (NT-proBNP) in patient whole blood samples using the LFSMIA and realized a strong linear correlation (r>0.99) with standard clinical lab results. These and other attributes of the present disclosure will be apparent upon a complete review of the specification, including the accompanying figures.

In some aspect, the present disclosure provides a method of detecting a target molecule. The method includes contacting a sample comprising the target molecule with a substrate that comprises a plurality of capture antibodies, or antigen binding portions thereof, that specifically bind to the target molecule to form captured target molecules, wherein the plurality of capture antibodies, or antigen binding portions thereof, are unlabeled; contacting the captured target molecules with a plurality of detection antibodies, or antigen binding portions thereof, that bind to the captured target molecules to form target molecule complexes, wherein the plurality of detection antibodies, or antigen binding portions thereof, are unlabeled; taking a series of dynamically tracked real-time images of the target molecule complexes over one or more selected periods of time to produce imaged target molecule complexes; and, quantifying an amount of target molecules in the sample using the imaged target molecule complexes, thereby detecting the target molecule.

In some embodiments, the detection antibodies, or antigen binding portions thereof, specifically bind to a first epitope of the target molecules, wherein the capture antibodies, or antigen binding portions thereof, specifically bind to a second epitope of the target molecules, and wherein the first and second epitopes differ from one another. In some embodiments, the target molecule complexes each comprise a single bound target molecule. In some embodiments, the quantifying step comprises filtering in terms of position, molecular weight, binding duration, and/or binding frequency of detected binding events in the imaged target molecule complexes, and determining and fitting a time course of a total count of specific binding of the detection antibodies, or antigen binding portions thereof, using a Gaussian Bayes algorithm. In some embodiments, the quantifying step comprises digitally counting the imaged target molecule complexes in the images to quantify the amount of target molecule in the sample. In some embodiments, the quantifying step comprises determining a concentration of the target molecule in the sample. In some embodiments, the target molecule is a compound selected from the group consisting of: an interleukin-6 (IL-6) molecule, a prostate-specific antigen (PSA) molecules, and a N-terminal pro b-type natriuretic peptide (NT-proBNP) molecule. In some embodiments, the sample comprises buffer, serum, and/or whole blood.

In some embodiments, the method includes flowing the captured target molecules through a plasma separator prior to contacting the captured target molecules with the plurality of detection antibodies, or antigen binding portions thereof. In some embodiments, the plurality of capture antibodies, or antigen binding portions thereof, are disposed on a surface of a solid support. In some embodiments, the method includes performing at least a portion of the method in a microfluidic device or system. In some embodiments, the method includes obtaining the sample from a subject. In some embodiments, the method includes administering, or discontinuing administering, therapy to the subject based at least in part on the amount of target molecule in the sample obtained from the subject. In some embodiments, the method includes generating a therapy recommendation for the subject based at least in part on the amount of target molecule in the sample obtained from the subject.

In another aspect, the present disclosure provides microfluidic device that comprises a body structure comprising at least one microfluidic channel disposed at least partially in the body structure; a sample inlet area disposed at least partially in the body structure and in fluid communication with the microfluidic channel, wherein the sample inlet area is configured to receive sample aliquots that comprise mixtures of substantially unprocessed target molecules and a plurality of detection antibodies, or antigen binding portions thereof, that specifically bind to the target molecules in the sample; an assay area disposed at least partially in the body structure and in fluid communication with the microfluidic channel; a plurality of capture antibodies, or antigen binding portions thereof, disposed on a surface of the assay area, wherein the capture antibodies, or antigen binding portions thereof, specifically bind to the target molecules when the target molecules are conveyed from the sample inlet area to the assay area through at least a portion of the microfluidic channel into contact with the plurality of capture antibodies, or antigen binding portions thereof, to form captured target molecules; and, wherein the detection antibodies, or antigen binding portions thereof, specifically bind to the target molecules in the captured target molecules when the detection antibodies, or antigen binding portions thereof, are conveyed from the sample inlet area to the assay area through at least a portion of the microfluidic channel into contact with the captured target molecules to form target molecule complexes; wherein the microfluidic device is configured to operably connect to a fluid conveyance mechanism that effects fluid conveyance through the microfluidic channel to and/or from the sample inlet area and the assay area; and wherein the microfluidic device is configured to operably interface with a detection mechanism that images the target molecule complexes in the assay area to produce imaged target molecule complexes such that a controller operably connected to the detection mechanism quantifies an amount of target molecule in the sample aliquots from the imaged target molecule complexes.

In some embodiments, the detection antibodies, or antigen binding portions thereof, specifically bind to a first epitope of the target molecules, wherein the capture antibodies, or antigen binding portions thereof, specifically bind to a second epitope of the target molecules, and wherein the first and second epitopes differ from one another. In some embodiments, the target molecule complexes each comprise a single bound target molecule. In some embodiments, the controller comprises a processor, and a memory communicatively coupled to the processor, the memory storing non-transitory computer executable instructions which, when executed on the processor, perform operations comprising: digitally counting the imaged target molecule complexes in the images to quantify the amount of target molecule in the sample aliquots. In some embodiments, the amount of target molecule in the sample aliquots comprises a concentration of the target molecule in the sample aliquots.

In some embodiments, the detection mechanism comprises a bright-field microscope. In some embodiments, a kit comprises the microfluidic device. In some embodiments, the sample inlet area comprises a sample inlet port. In some embodiments, a microfluidic chip or cartridge comprises the microfluidic device. In some embodiments, a point-of-care device or system comprises or is configured to receive the microfluidic device. In some embodiments, the target molecule is a compound selected from the group consisting of: an interleukin-6 (IL-6) molecule, a prostate-specific antigen (PSA) molecules, and a N-terminal pro b-type natriuretic peptide (NT-proBNP) molecule. In some embodiments, the sample comprises buffer, serum, and/or whole blood. In some embodiments, a system comprises the microfluidic device.

In another aspect, the present disclosure provides a computer readable media comprising non-transitory computer executable instruction which, when executed by at least electronic processor, perform at least: taking a rolling average of a raw image sequence to remove background noise to produce denoised images; converting the denoised images to probability images; identifying candidate pixels of a binding event in one or more frames of the probability images to produce identified candidate pixels; fitting a Gaussian function pixels of the denoised images with the same coordinates as the identified candidate pixels to produce a Gaussian fitting; filtering out invalid binding events and determining positions and intensities of valid binding events based on the Gaussian fitting to produce detected binding events; and, mapping the detected binding events in all frames of the denoised images over time with respect to spatial locations of the detected binding events.

### BRIEF DESCRIPTION OF DRAWINGS

FIG. 1 is a flow chart that schematically shows exemplary method steps of detecting target molecules in samples according to some aspects disclosed herein.

FIG. 2 is a schematic diagram of an exemplary point-of-care device and microfluidic device suitable for use with certain aspects disclosed herein.

FIG. 3 is a schematic diagram of an exemplary system suitable for use with certain aspects disclosed herein.

FIGS. 4A and 4B. The principle of LFSM-immunoassay. (a) Experimental setup. The analytes of interest are first pulled down by the capture antibodies tethered to the gold film via an alkane linker. A P-polarized incident light is focused on the gold surface with an incident angle of 71° to excite SPR and the associated evanescent field. After introducing detection antibody solution, the process of detection antibody binding to the biomarkers caught on the surface is recorded by a CMOS camera via an air objective above the microfluidic channel. (b) The raw images are processed by a custom-written algorithm to remove background noise and extract the binding events of single particles hitting the sensor surface. A time course of the total count of the specific binding of detection antibody is then determined and fitted with Gaussian Bayes algorithm to enhance the precision after filtering in terms of the position, molecular weight, binding duration, and binding frequency of the detected binding events is applied. Finally, a standard curve (binding events vs analyte concentration) is generated from triplicate tests for different analyte concentrations.

FIGS. 5A-5E. Validation of LFSM-immunoassay. (a) Workflow of LFSM-immunoassay. Capture antibodies were first immobilized on the gold film through alkane linker. Analyte samples were flowed through the microfluidic channel for 10 minutes in NT-proBNP detection or 2 hours in the other cases. 10 nM detection antibody solution was injected for 10 minutes and the binding of detection antibody to analytes captured on the sensor surface was recorded. (b) The relationship between PSM image intensity and the molecular weight (MW) of particles, determined from PSM images of different proteins in PBS solution. The mean intensity of one type of protein is the mean of its Gaussian fit to the corresponding histograms in FIG. 11. The error bars are the model fitting error in FIG. 11. The number of total binding events over time for different IL-6 concentrations in (c) pure buffer and (d) bovine serum. For clarity, the fitted results of Gaussian Bayes algorithm (solid line) and the standard deviation of original data (shadow) were plotted. (e) Standard curves of IL-6 detection in pure buffer (darker grey) and bovine serum (lighter grey). The error bars were s.d. calculated by Gaussian Bayes algorithm. The blue and yellow dashed line represent the limit of detection for pure buffer and bovine serum respectively, defined as mean+3×s.d. determined by Gaussian Bayes algorithm of blank solution.

FIGS. 6A-6D. Rapid NT-proBNP detection in human plasma and clinical evaluation. (a) Temporal profiles for different NT-proBNP concentrations in human plasma. For clarity, the fitted results of Gaussian Bayes algorithm (solid line) and the standard deviation of original data (shadow) were plotted. (b) Standard curve of NT-proBNP detection in human plasma. The error bars were s.d. calculated by Gaussian Bayes algorithm. The dashed line represents the limit of detection for NT-proBNP in human plasma, defined as mean+3×s.d. determined by Gaussian Bayes algorithm of blank solution (horse serum). (c) Pearson's correlation between LFSM-immunoassay and Roche's Elecsys proBNP II assay. The results were determined from measurements of serum samples from 28 patients using both the Elecsys proBNP II assay and LFSM-immunoassay. (d) Summary of all the measurement results for the 28 patients.

FIGS. 7A-7D. Protein detection in whole blood. Time courses of total binding events for different concentrations of (a) IL-6 and (c) PSA in bovine whole blood. For clarity, the fitted results of Gaussian Bayes algorithm (solid line) and the standard deviation of original data (shadow) were plotted. Standard curve of (b) IL-6 and (d) PSA in bovine whole blood. The error bars were s.d. calculated by Gaussian Bayes algorithm. The red dashed line represents the limit of detection, defined as mean+3×s.d. determined by Gaussian Bayes algorithm of blank solution.

FIGS. 8A-8F. Point-of-Care LFSMiA and its clinical evaluation. (a) Photograph of LFSMiA-lite. (b) and (d) are dynamic response profiles for different concentration of IL-6 in horse serum and NT-proBNP in bovine whole blood, respectively. For clarity, the fitted results of Gaussian Bayes algorithm (solid line) and the standard deviation of original data (shadow) were plotted. Stand curves of IL-6 detection in horse serum (c) and NT-proBNP detection in bovine whole blood (e). The error bars were s.d. calculated by Gaussian Bayes algorithm. The dashed line represents the limit of detection, defined as mean+3×s.d. determined by Gaussian Bayes algorithm of blank solution. (f) Linear regression plot of NT-proBNP concentration in patient's whole blood samples determined by LFSMiA-lite and its corresponding serum samples measured using Roche's Elecsys proBNP II assay.

FIGS. 9A-9E. Improvements of Gaussian process model. (a) Comparison of the detection limit with and without processed by the GP model under different samples and conditions. (b), (c) and (d) shows the coefficient of variation of IL-6 detection in pure buffer, horse serum and bovine whole blood determined with and without GP model, respectively. (e) presents the improvement of GP model on CV under different conditions.

FIG. 10. The setup for LFSM-immunoassay. (a) Optical configuration of plasmonic scattering microscopy used for LFSM-immunoassay. The light from laser is collected and collimated by a 20× objective. A lens group is used to resize the collimated light in order to increase light intensity. The incident light is directed and focused on the prism surface with an incident angle of 71° to excite SPR by a reflector and a short-focus lens. The focal lengths for the lenses are f₁=200 mm, f₂=30 mm and f₃=30 mm. The intensity of incident light is up to 2000 W/cm². The scattered light from the biomolecule and gold surface is collected by a 60× air objective (Olympus, LUCPLFLN60X, NA=0.7) equipped with a 180 mm tube lens to form an image on a CMOS camera (MQ013MG-ON, XIMEA). (b) The microfluidic channel assembly. A 50 μm thick double-sided tape with a 3×36 mm² straight channel is sandwiched between a cover glass and a gold film. The gold film is pre-modified with alkane linker and spacer. The cover glass has 2 drilled holes located at the 2 ends of the straight channel, and two PDMS pieces each with a through hole are adhered to the cover glass to form the inlet and outlet of the microfluidic channel. The microfluidic channel assembly is sealed with epoxy glue.

FIG. 11. Calibration data of PSM mass detection. To determine the relationship between PSM image intensity and protein molecular weight, 5 different proteins with known molecular weight were dissolved in PBS buffer and flowed over bare gold surface. Binding events of different proteins were recorded and extracted to obtain histograms of their image intensity. The solid lines are the Gaussian fitting results for the 5 proteins. The incident light intensity was 2 kW/cm². The exposure time was 1 ms for IgM, 2 ms for IgA, 5 ms for IgG, 10 ms for BSA and 15 ms for cTnT. All the results were normalized to exposure time of 10 ms.

FIG. 12. Complete standard curve of IL-6 detection in pure buffer, where the error bars were calculated from s.d. of triplicate tests. The dashed line is the limit of detection for IL-6 detection in pure buffer, defined as mean+3×s.d. counts of blank solution.

FIGS. 13A-13I. Temporal profiles of total binding events for the 3 replicate tests of different IL-6 concentrations in pure buffer. (a-i) The real-time counting results of total binding events for IL-6 spiked pure buffer with concentrations of 0, 21 fg/mL, 210 fg/mL, 2.1 pg/ml, 21 pg/mL, 210 pg/mL, 2.1 ng/ml, 21 ng/mL and 210 ng/ml respectively.

FIGS. 14A-14F. Time courses of total binding events for triplicate tests of different IL-6 concentrations in bovine serum. (a-f) The real-time counting results of total binding events for IL-6 spiked bovine serum with concentrations of 0, 21 fg/mL, 210 fg/mL, 2.1 pg/ml, 21 pg/mL and 210 pg/mL respectively.

FIGS. 15A-15F. Real-time counting results of total binding events for generating NT-proBNP standard curve corresponding to (a) blank buffer (horse serum), NT-proBNP spiked human plasma with concentrations of (b) 16.7 pg/mL, (c) 93.2 pg/mL, (d) 850 pg/ml, (e) 8.5 ng/ml and (f) 85 ng/mL.

FIGS. 16A-16F. Raw calibration data of IL-6 detection in bovine whole blood. (a-f) The real-time counting results of IL-6 spiked bovine whole blood with concentrations of 0, 21 fg/mL, 210 fg/mL, 2.1 pg/ml, 21 pg/mL and 210 pg/mL.

FIGS. 17A-17F. Raw calibration data of PSA detection in bovine whole blood. (a-f) The real-time counting results of PSA spiked bovine whole blood with concentrations of 0, 32 fg/mL, 320 fg/mL, 3.2 pg/ml, 32 pg/mL and 320 pg/mL.

FIG. 18. The standard curve for the detection of IL-6 spiked into bovine whole blood without passing through the binding event filters of our real-time counting algorithm. The dashed line indicates the limit of detection, defined as the mean plus three times s.d. of the counts of blank control without spiked analyte. (Details of each data point are shown in FIG. 19).

FIGS. 19A-19F. Total number of binding events over time for the data points in FIG. 18. The concentrations of IL-6 corresponding to each figure are the same as those in FIG. 16.

FIG. 20. Automatic algorithm for real-time counting of detection antibody specific binding. Raw image sequence was rolling averaged to remove the background noise. Then the denoised images were converted to probability images, from which candidate pixels of a binding event in a certain frame were identified. A Gaussian function was fitted to the pixels of the denoised images with the same coordinates as the identified candidate pixels. Based on the Gaussian fitting, invalid binding events were filtered out and the position and intensity of valid binding events were determined. The detected binding events in all the frames were mapped over time with respect to their spatial locations. Binding events from consecutive frames were deemed as binding of an individual detection antibody if their coordinates were within diffraction limit distance from each other. Finally, the temporal profile of total binding events of detection antibody was determined after nonspecific binding or surface impurities were filtered out.

FIGS. 21A and 21B. Binding event of single protein imaged with PSM. Representative images at different time points of a binding event were shown in the top panels (scale bars, 1 μm). (a) The corresponding image intensity profiles of the dashed lines shown in the top panels. (b) Corresponding maximum intensities (dark dot) of the binding particle extracted by Gaussian fitting and linear fit to the intensity time course (solid line). The result is consistent with previous research which shows that the intensity of binding would grow and fade.

FIGS. 22A-22D. Standard curves of IL-6 detection in pure buffer corresponding to different binding frequency thresholds. The threshold used are (a) 1, (b) 2, (c) 5 and (d) 10 times in ten mins (#/10 mins), respectively. The dashed lines indicate the LOD (mean+3×s.d. of the count of blank buffer). The error bars are s.d. of triplicate tests. The Details about each standard curve are shown in Table 1.

FIGS. 23A-23D. Standard curves of IL-6 detection in bovine whole blood corresponding to different binding frequency thresholds (Same set of thresholds as in FIG. 22). Details of each standard curve are shown in Table 2.

FIG. 24. Real-time counting of binding and unbinding events. Time courses of detection antibody binding to IL-6 captured by the sensor surface represented as the counting of binding events (solid line) and unbinding events (dash line). The experiments were repeated three times under the same conditions.

FIGS. 25A-25D. Influence of detection antibody's concentration on NT-proBNP detection. The number of binding events vs binding time of (a) blank buffer and (b) 100 fM NT-proBNP in human plasma. For clarity, the averaged curves of triplicate tests of (c) 50 nM detection antibody and (d) 10 nM detection antibody were plotted in (a).

FIGS. 26A and 26B. Investigation of effect of endogenous NT-proBNP in human blood. (a) Conventional ELISA measurement of the endogenous NT-proBNP level in the normal human plasma used to generate LFSM-immunoassay standard curve. Recombinant NT-proBNP was spiked into horse serum with final concentrations of 25, 50, 100 and 200 μg/mL. The spiked horse serum standard solutions were measured by conventional ELISA and the corresponding absorbance readings were used to obtain the linear standard curve fit (r-square=0.996). The dashed line is the LOD of NT-proBNP in horse serum by ELISA. Yellow star represents the measurement of endogenous NT-proBNP level. (b) LFSM-immunoassay measurement of the endogenous NT-proBNP level in the normal human plasma. Star represents the endogenous NT-proBNP measurement on the LFSM-immunoassay standard curve.

FIGS. 27A-27C. NT-proBNP detection in human plasma with 5-min counting time. The same set of raw data as in FIG. 3 was processing in the same way to plot this figure except that only the first 5 mins was considered here. (a) 5-min temporal profiles for different NT-proBNP concentrations in human plasma. (b) Standard curve of NT-proBNP detection in human plasma for 5-min counting time. (c) Pearson's correlation between LFSM-immunoassay and Roche's Elecsys proBNP II assay using 5-min counting time.

FIGS. 28A-28F. Raw 5-min temporal profiles of triplicate tests corresponding to (a) blank buffer (horse serum), NT-proBNP spiked human plasma with concentrations of (b) 16.7 pg/mL, (c) 93.2 pg/mL, (d) 850 pg/ml, (e) 8.5 ng/ml and (f) 85 ng/ml.

FIGS. 29A-29D. Calibration data of LFSMiA-lite mass detection. To determine the relationship between image intensity and protein molecular weight, 4 different proteins with known molecular weight were dissolved in PBS buffer and flowed over bare gold surface. Binding events of different proteins were recorded and extracted to obtain histograms of their image intensity. The solid lines are the Gaussian fitting results for the 4 proteins. The incident light intensity was 2 kW/cm². The exposure time was 2.5 ms for IgM, 5 ms for IgA, 10 ms for IgG, 15 ms for BSA. All the results were normalized to exposure time of 15 ms.

FIGS. 30A-30F. Raw calibration data of IL-6 detection in bovine whole blood on LFSMiA-lite. (a-f) The real-time counting results of IL-6 spiked bovine whole blood with concentrations of 0, 21 fg/mL, 210 fg/mL, 2.1 pg/ml, 21 pg/mL and 210 pg/mL.

FIGS. 31A-31F. Real-time counting results of total binding events on LFSMiA-lite for generating NT-proBNP standard curve corresponding to (a) blank buffer (bovine whole blood), NT-proBNP spiked bovine whole blood with concentrations of (b) 8.5 pg/mL, (c) 85.0 pg/mL, (d) 850 pg/ml, (e) 8.5 ng/ml and (f) 85 ng/mL.

FIGS. 32A-321. Improvements of Gaussian process model. (a), (d) and (g) are the standard curves of IL-6 detection in pure buffer, horse serum and bovine whole blood determined by the total counts in 10 mins, respectively. (b), (e) and (h) are the corresponding standard curves processed by GP model. (c), (f) and (i) show the improvements of GP model on the CV of IL-6 detection in pure buffer, horse serum and bovine whole blood, respectively.

FIGS. 33A-33F. Improvements of Gaussian process model. (a) and (d) are the standard curves of NT-proBNP detection in human plasma and PSA detection in bovine whole blood determined by the total counts in 10 mins. (b) and (e) are the corresponding standard curves processed by GP model. (c) and (f) show the improvements of GP model on the CV of NT-proBNP detection in human plasma and PSA detection in bovine whole blood, respectively.

FIGS. 34A-34F. Improvements of Gaussian process model. (a) and (d) are the standard curves of IL-6 detection in horse serum and NT-proBNP detection in bovine whole blood on LFSMiA-lite determined by the total counts in 10 mins. (b) and (e) are the corresponding standard curves processed by GP model. (c) and (f) show the improvements of GP model on the CV of IL-6 detection in horse serum and NT-proBNP detection in bovine whole blood on LFSMiA-lite, respectively.

FIG. 35. Sensor surface functionalization. The gold substrate was first dipped in an ethanol solution with 100 μM 8-mercaptooctanoic acid (MOA) and 10 mM 6-mercapto-1-hexanol (MCH) overnight. After rinsed by ethanol and DI water for three times, 60 μL EDC and NHS mixed solution (5 mM and 10 mM in DI water) was injected 3 times into the microfluidic channel at 0, 5 and 10 min in a 15-min total incubation time. The sensor was then cleaned by 200 μL MOPS buffer. Capture antibody with a concentration of 100 μg/mL in MOPS buffer was injected three times to improve the efficiency of surface modification. The capture antibody coated sensor was then quenched by 1 M ethanolamine with pH of 9.6.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and devices, the following terminology, and grammatical variants thereof, will be used in accordance with the definitions set forth below.

About. As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent (e.g., an immunological therapeutic agent) to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Antigen Binding Portion: As used herein, the term “antigen binding portion” refers to a portion of an antibody that specifically binds to a target molecule, e.g., a molecule in which one or more immunoglobulin chains is not full length, but which specifically binds to a target molecule. Examples of binding portions encompassed within the term “antigen-binding portion of an antibody include (i) a Fab fragment, a monovalent fragment consisting of the VLC, VHC, CL and CH1 domains: (ii) a F(ab′)² fragment, a bivalent fragment comprising two Fab fragments linked by a disulfide bridge at the hinge region; (iii) a Fd fragment consisting of the VHC and CH1 domains; (iv) a Fv fragment consisting of the VLC and VHC domains of a single arm of an antibody, (v) a dAb fragment, which consists of a VHC domain; and (vi) an isolated complementarity determining region (CDR) having sufficient framework to specifically bind, e.g., an antigen binding portion of a variable region. An antigen binding portion of a light chain variable region and an antigen binding portion of a heavy chain variable region, e.g., the two domains of the Fv fragment, VLC and VHC, can be joined, using recombinant methods, by a synthetic linker that enables them to be made as a single protein chain in which the VLC and VHC regions pair to form monovalent molecules (known as single chain Fv (scFV)). Such single chain antibodies are also encompassed within the term “antigen binding portion” of an antibody. The term “antigen binding portion” encompasses a single-domain antibody (sdAb), also known as a “nanobody” or “VHH antibody,” which is an antibody fragment consisting of a single monomeric variable antibody domain. These antibody portions are obtained using conventional techniques known to those with skill in the art, and the portions are screened for utility in the same manner as are intact antibodies.

Binding: As used herein, the term “binding” or “binding interaction”, typically refers to a non-covalent association between or among two or more entities. “Direct” binding involves physical contact between entities or moieties; “indirect” binding involves physical interaction by way of physical contact with one or more intermediate entities. Binding between two or more entities can be assessed in any of a variety of contexts—including where interacting entities or moieties are studied in isolation or in the context of more complex systems (e.g., while covalently or otherwise associated with a carrier entity and/or in a biological system or cell).

Binding Moiety: As used herein, the term “binding moiety” refers to a portion of a chemical compound or structure that selectively or preferentially binds to another chemical compound or structure. In some embodiments, for example, a surface (e.g., a solid surface of a microfluidic cavity or channel) is functionalized or conjugated with binding moieties (e.g., capture antibodies or antigen binding portions thereof), which selectively or preferentially bind to target molecules.

Data set. As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Detect: As used herein, the term “detect,” “detecting,” or “detection” refers to an act of determining the existence or presence of one or more target analytes (e.g., a target molecule in a sample.

Epitope: As used herein, “epitope” refers to the part of an antigen to which an antibody and/or an antigen binding portion binds.

N-terminal prohormone B-type natriuretic peptide: As used herein, the term “N-terminal prohormone B-type natriuretic peptide” or “NT-proBNP” refers to a prohormone with a 76 amino acid N-terminal inactive protein that is cleaved from the molecule to release B-type natriuretic peptide (BNP, also known as brain natriuretic peptide 32).

Sample: As used herein, “sample” or “fluidic sample” refers to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide), which is assayed as described herein. A sample may also be any body fluid or excretion (for example, but not limited to, blood, urine, stool, saliva, tears, bile) that contains cells, cell components, or non-cellular fractions.

Subject. As used herein, “subject” refers to an animal, such as a mammalian species (e.g., human, dog, cat) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). In certain embodiments, the subject is a human. In certain embodiments, the subject is a companion animal, including, but not limited to, a dog or a cat. A subject can be a healthy individual, an individual that has or is suspected of having a disease or a predisposition to the disease, or an individual that is in need of therapy or suspected of needing therapy. The terms “individual” or “patient” are intended to be interchangeable with “subject.”

System: As used herein, “system” in the context of analytical instrumentation refers a group of objects and/or devices that form a network for performing a desired objective.

### DETAILED DESCRIPTION

In some aspects, the present disclosure provides a real-time label-free single-molecule immunoassay (LFSMiA) based on mass imaging, which enables ultra-sensitive and direct protein detection in neat blood sample matrices. This novel LFSMiA employs plasmonic scattering microscopy (PSM), a technique capable of detecting scattered light from single molecules based on their mass property, to monitor the formation of individual biomarker immunocomplexes on the sensor surface. In some embodiments, a 2-step sandwich assay format is adopted in the LFSMiA to separate sample incubation from detection antibody binding. In this way, a sample in a complex matrix can be directly incubated with the capture antibody functionalized sensor surface and the sample matrix is washed away at the end of incubation to reduce background signal. As such, PSM can specifically extract and track the binding of single detection antibody in pure buffer to the biomarker molecules captured on the sensor surface. Moreover, the LFSMiA exhibits reduced susceptibility to matrix effects, such as interference from molecules structurally similar to the biomarker or heterophilic antibodies, by dynamically tracking binding events and utilizing binding kinetics data to distinguish between specific and nonspecific interactions. In some embodiments, we also use a Gaussian Bayes algorithm to fit the real-time binding data, providing improved detection accuracy. Additionally, the label-free nature of this assay eliminates complications associated with labeling reagents, which can hinder binding efficiency and lead to nonspecific interactions. To the best of our knowledge, the LFSMiA is also the first technology to realize label-free detection of protein biomarkers at single-molecule level. To validate this novel method, we have demonstrated detection of interleukin-6 (IL-6, an important infection and inflammation biomarker) and prostate-specific antigen (PSA, a widely used prostate cancer biomarker) in both pure buffer and neat serum. We also evaluated the clinical application of LFSMiA with N-terminal pro b-type natriuretic peptide (NT-proBNP), an important biomarker for heart failure, and demonstrated excellent linear correlation with clinical lab results measured with the commercial Elecsys proBNP II assay. To highlight the technology's potential for point-of-care diagnostics, we developed an inexpensive and miniaturized version of PSM microscope, which achieved comparable assay performance. Our method is also the first to realize sub-femtomolar biomarker detection in undiluted whole blood sample. Therefore, we believe that the LFSMiA will be a promising method of revolutionizing in vitro molecular diagnostics. These and other attributes will be apparent upon a complete review of this specification, including the accompanying figures.

In some embodiments, the assays of the present disclosure can be done at low concentrations of biomarker (e.g., femtomolar), are ultrasensitive and involve the direct detection of protein biomarkers. In some embodiments, the assays of the present disclosure involve label-free single molecule detection, which eliminates the cost and issues of labeling. In some embodiments, the present disclosure provides imaging based sandwich assays, can be tested in whole blood, and show strong linear correlation with current technologies and their clinical lab reported value for biomarkers in serum samples.

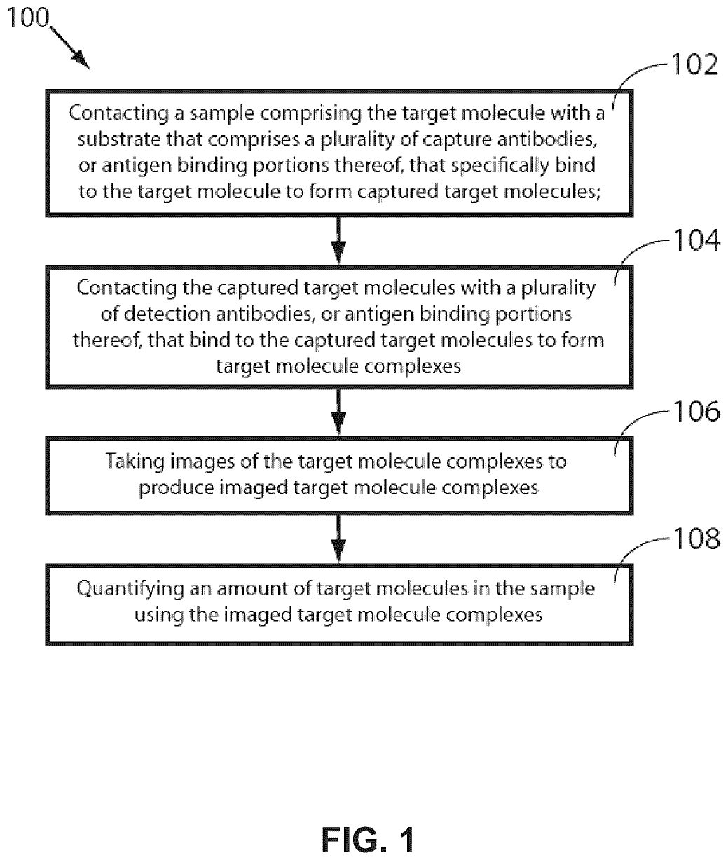

To illustrate, FIG. 1 is a flow chart that schematically shows exemplary method steps of detecting target molecules. As shown, method 100 includes contacting a sample comprising the target molecule with a substrate that comprises a plurality of capture antibodies, or antigen binding portions thereof, that specifically bind to the target molecule to form captured target molecules (step 102). Method 100 also includes contacting the captured target molecules with a plurality of detection antibodies, or antigen binding portions thereof, that bind to the captured target molecules to form target molecule complexes (step 104). As additionally shown, method 100 also includes taking images of the target molecule complexes to produce imaged target molecule complexes (step 106) and quantifying an amount of target molecules in the sample using the imaged target molecule complexes (step 108).

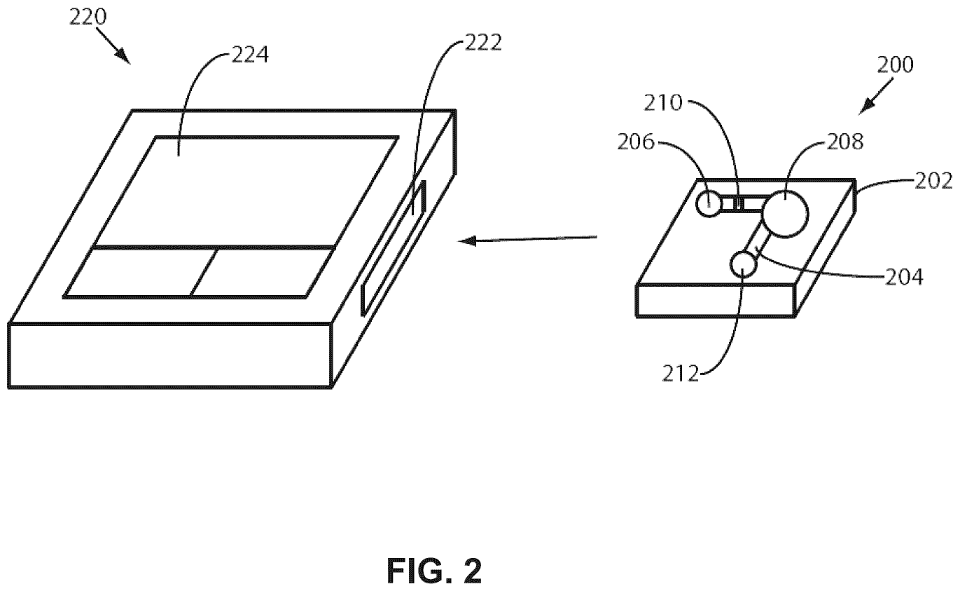

To further illustrate, FIG. 2 is a schematic diagram of an exemplary point-of-care device 220 and microfluidic device 200 (shown as, a microfluidic chip or cartridge) suitable for use with certain aspects disclosed herein. As shown, device 200 includes body structure 202 that includes microfluidic channel 204 disposed at least partially in body structure 202. Device 200 also includes sample inlet area 206 (shown as including a sample inlet port) disposed at least partially in body structure 202 and in fluid communication with microfluidic channel 204. Sample inlet area 206 is configured to receive sample aliquots that include mixtures of substantially unprocessed target molecule-containing whole blood or other sample type and a plurality of detection antibodies, or antigen binding portions thereof, that each comprise a first recognition moiety, which detection antibodies, or antigen binding portions thereof, specifically bind to the target molecules in the whole blood to form detection target molecule complexes. As shown, device 200 also includes assay area 208 disposed at least partially in body structure 202 and in fluid communication with microfluidic channel 204. Device 200 also includes plasma separator 210 disposed in a microfluidic channel between sample inlet area 206 and assay area 208.

As described herein, a plurality of capture antibodies, or antigen binding portions thereof, is typically disposed on a surface of assay area 208 in which the capture antibodies, or antigen binding portions thereof, specifically bind to the target molecules when the target molecules are conveyed from sample inlet area 206 to assay area 208 through at least a portion of a microfluidic channel through optional plasma separator 210 into contact with the plurality of capture antibodies, or antigen binding portions thereof, to form captured target molecules. As additionally shown, device 200 also includes optional nanoparticle (NP) reservoir 212 disposed at least partially in body structure 202 and in fluid communication with microfluidic channel 204. In some embodiments, NP reservoir 212 is configured to contain NPs that each comprise a second recognition moiety that binds to the first recognition moiety of the detection antibodies, or antigen binding portions thereof, of the captured target molecules when the NPs are conveyed from NP reservoir 212 to assay area 208 through at least a portion of microfluidic channel 204 into contact with the captured target molecules to form captured NP-target molecule complexes.

As additionally shown in FIG. 2, point-of-care (POC) device 220 is configured to receive device 200 in device receiving area 222. POC device 220 is also shown as including display screen 224 for inputting instructions and viewing assay results. Although not within view, device 200 is configured to operably connect to a fluid conveyance mechanism disposed in POC device 220 that effects fluid conveyance through the microfluidic channels of device 200 to and/or from sample inlet area 206, assay area 208, and optional NP reservoir 212. Although additionally not within view, device 200 is also configured to operably interface with a detection mechanism (e.g., a camera, a microscope, or other imaging device) disposed in POC device 220 that images the target molecule complexes in assay area 208 to produce imaged target molecule complexes such that a controller that is also disposed in POC device 220 and operably connected to the detection mechanism quantifies an amount of target molecule in the sample aliquots from the imaged target molecule complexes. Typically, the controller comprises a processor, and a memory communicatively coupled to the processor, the memory storing non-transitory computer executable instructions which, when executed on the processor, perform operations that include digitally counting the imaged target molecule complexes in the images to quantify the amount of target molecule in the sample aliquots.

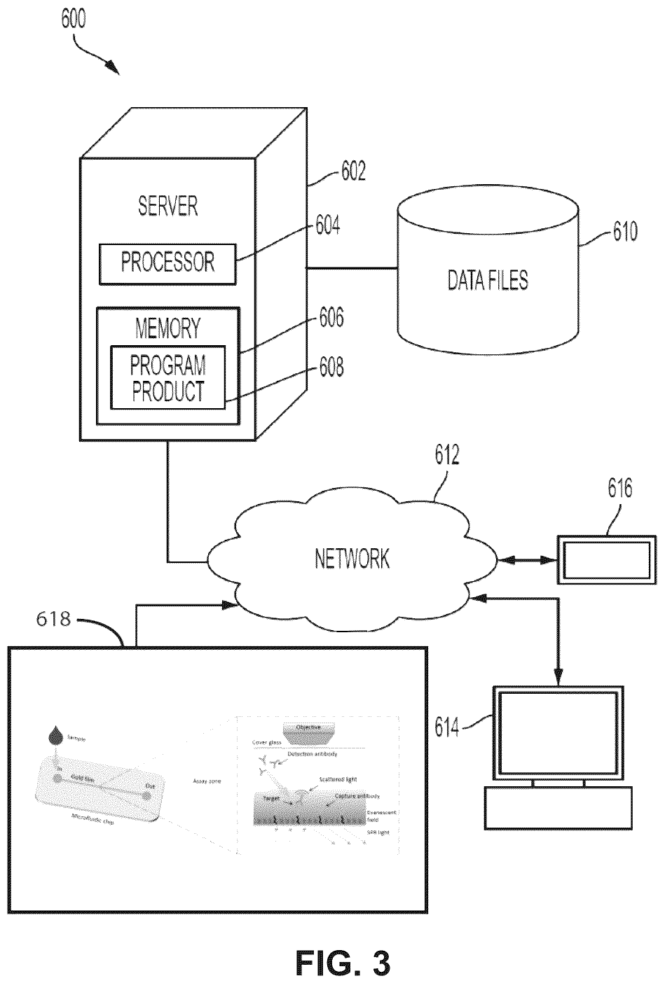

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 3 provides a schematic diagram of an exemplary system suitable for use with implementing at least aspects of the methods disclosed in this application. As shown, system 600 includes at least one controller or computer, e.g., server 602 (e.g., a search engine server), which includes processor 604 and memory, storage device, or memory component 606, and one or more other communication devices 614, 616, (e.g., client-side computer terminals, telephones, tablets, laptops, other mobile devices, etc. (e.g., for receiving imaging data sets or results, etc.) in communication with the remote server 602, through electronic communication network 612, such as the Internet or other internetwork. Communication devices 614, 616 typically include an electronic display (e.g., an internet enabled computer or the like) in communication with, e.g., server 602 computer over network 612 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 600 also includes program product 608 (e.g., for quantifying amounts of target molecule in sample aliquots using imaged target molecule complexes as described herein) stored on a computer or machine readable medium, such as, for example, one or more of various types of memory, such as memory 606 of server 602, that is readable by the server 602, to facilitate, for example, a guided search application or other executable by one or more other communication devices, such as 614 (schematically shown as a desktop or personal computer). In some aspects, system 600 optionally also includes at least one database server, such as, for example, server 610 associated with an online website having data stored thereon (e.g., entries patient data sets, etc.) searchable either directly or through search engine server 602. System 600 optionally also includes one or more other servers positioned remotely from server 602, each of which are optionally associated with one or more database servers 610 located remotely or located local to each of the other servers. The other servers can beneficially provide service to geographically remote users and enhance geographically distributed operations.

As understood by those of ordinary skill in the art, memory 606 of the server 602 optionally includes volatile and/or nonvolatile memory including, for example, RAM, ROM, and magnetic or optical disks, among others. It is also understood by those of ordinary skill in the art that although illustrated as a single server, the illustrated configuration of server 602 is given only by way of example and that other types of servers or computers configured according to various other methodologies or architectures can also be used. Server 602 shown schematically in FIG. 3, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 600. As also understood by those of ordinary skill in the art, other user communication devices 614, 616 in these aspects, for example, can be a laptop, desktop, tablet, personal digital assistant (PDA), cell phone, server, or other types of computers. As known and understood by those of ordinary skill in the art, network 612 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 608 is optionally in the form of microcode, programs, cloud computing format, routines, and/or symbolic languages that provide one or more sets of ordered operations that control the functioning of the hardware and direct its operation. Program product 608, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 608 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 608 is optionally copied from the computer-readable medium to a hard disk or a similar intermediate storage medium. When program product 608, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 608 includes non-transitory computer-executable instructions which, when executed by electronic processor 604, perform at least: conveying fluid through a microfluidic channel of an device as described herein to and/or from a sample inlet area and an assay area using a fluid conveyance mechanism; taking images of target molecule complexes in an assay area to produce imaged target molecule complexes using a detection mechanism; and quantifying an amount of target molecule in sample aliquots using imaged target molecule complexes.

Typically, imaging is obtained using microfluidic device or subassembly 218. As shown, microfluidic device or subassembly 218 includes a fluid conveyance mechanism for flowing samples and other reagents through microfluidic channels, as described herein. Target molecule complexes are counted using the optical imaging system shown, which includes an objective lens and a CCD camera.

### Example: Label-Free Single-Molecule Immunoassay

### Results

### Principle and Setup

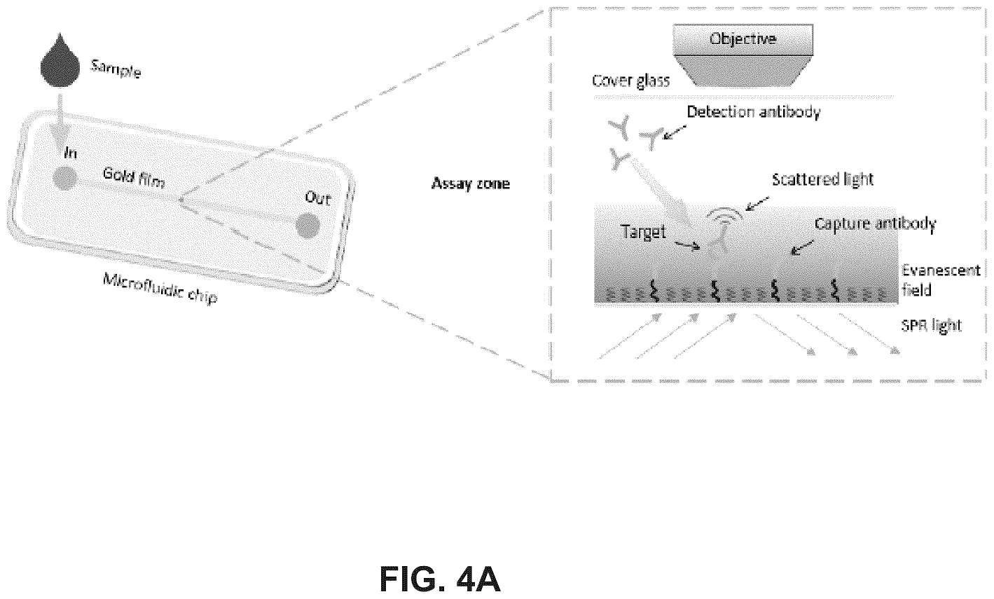

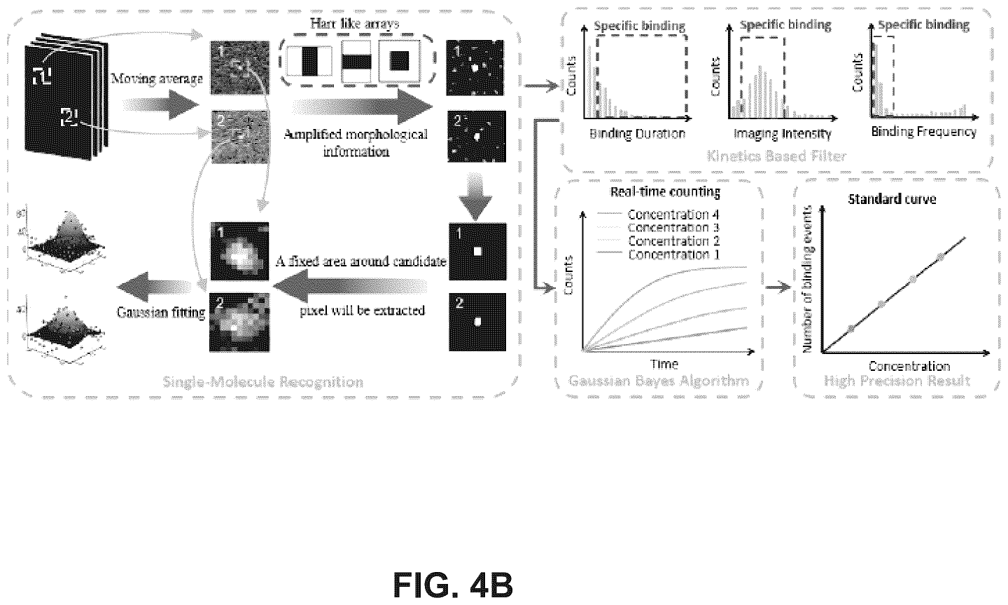

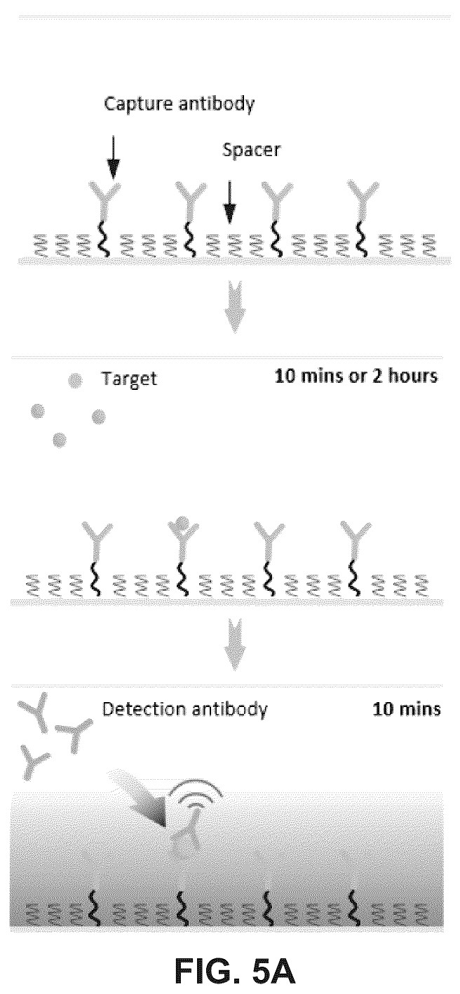

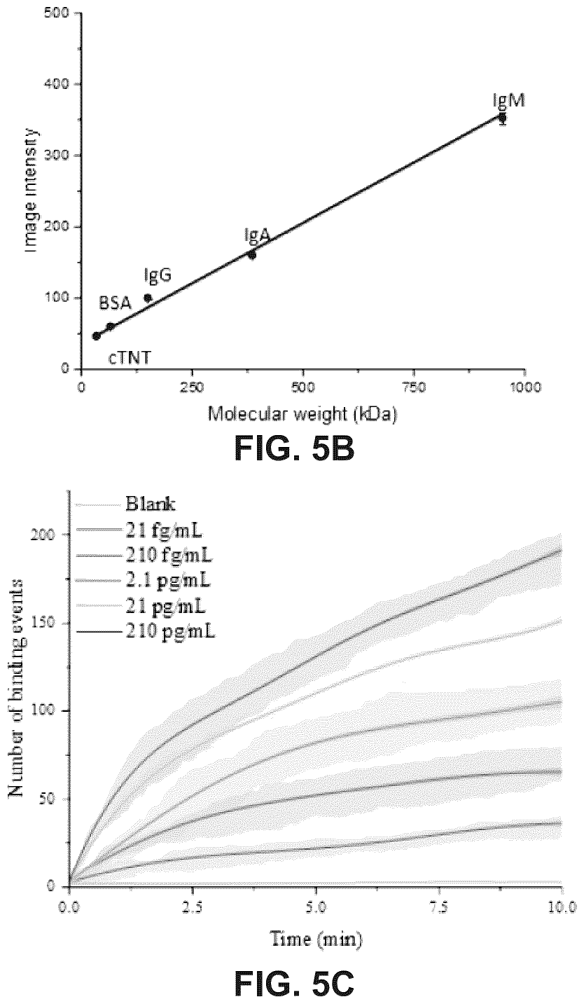

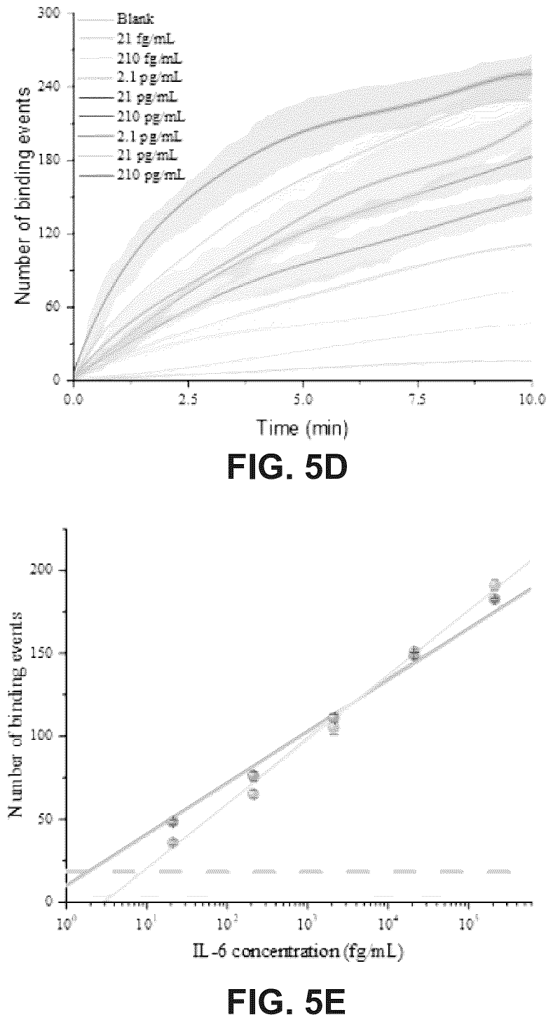

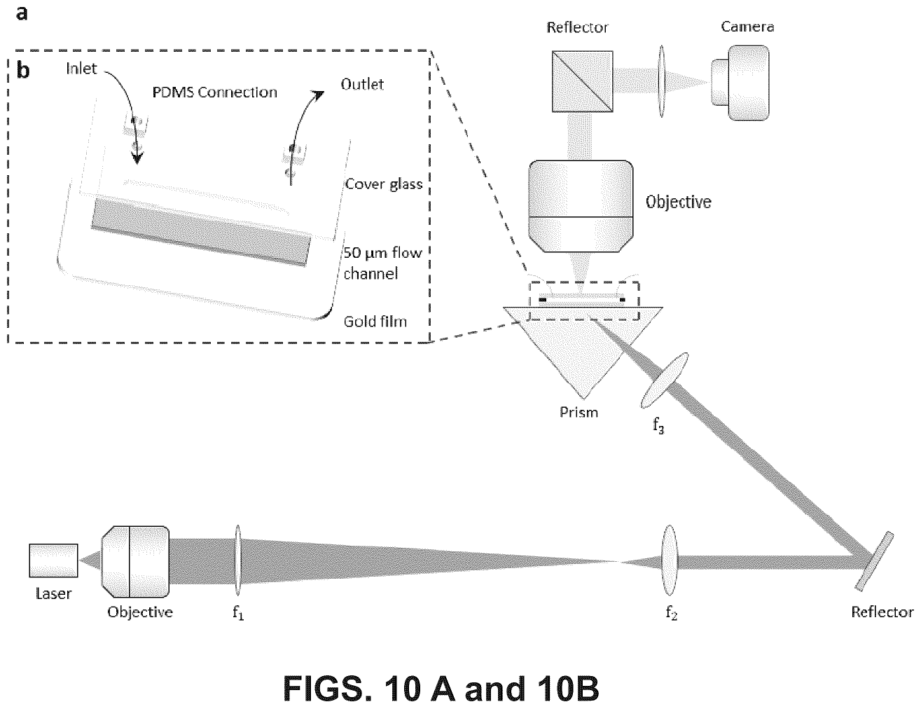

In LFSM-immunoassay, individual biomarkers of interest were pulled down by a capture antibody which was immobilized on a gold-coated glass slide via a polyethylene glycol (PEG) linker (FIG. 4a). The surface density between the PEG linker and spacer was modified to avoid steric hindrance effect (FIG. 5a). Detection antibodies were then introduced to bind with the biomarkers caught by the antibody, and the binding process was read out by plasmonic scattering microscopy (PSM) whose configuration was shown in FIG. 10. Briefly, p-polarized light from a laser was focused on the gold surface to excite surface plasmon resonance (SPR) and the associated evanescent field via a prism. The scattered light within the field was collected by an objective placed on top of the microchip and imaged with a COMS camera, which recorded the dynamic binding process of single detection antibody with a temporal resolution of ˜10 ms (FIG. 4a, FIG. 4b). Single binding event was tracked using an optimized data analysis approach, which provided high quality images and precise information of the single molecule hitting the surface. Based on the position, duration and intensity of each individual binding event, the amount of detection antibody specifically binding to the capture antibody-biomarker complex was counted over time. Viewing the binding event as a random event over time, stochastic process modeling is a natural choice to recover the random binding event at any time point. Considering that the binding counts change continuously with time and also present dependence across time, we proposed using a Gaussian process (GP) model to incorporate the dependency between binding counts to improve the estimation accuracy. A Bayesian hierarchical model built on the Gaussian process was used to fit the real-time binding counts (as described herein), and the estimated value of the binding counts were provided associated with their quantified standard deviations, Then the standard curve for measuring the biomarker concentration was obtained (FIG. 4b).

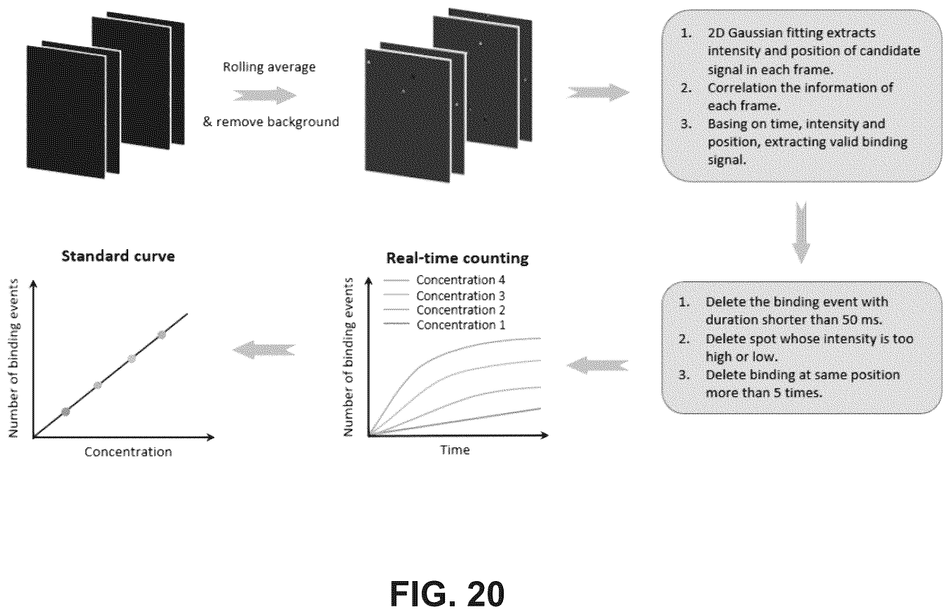

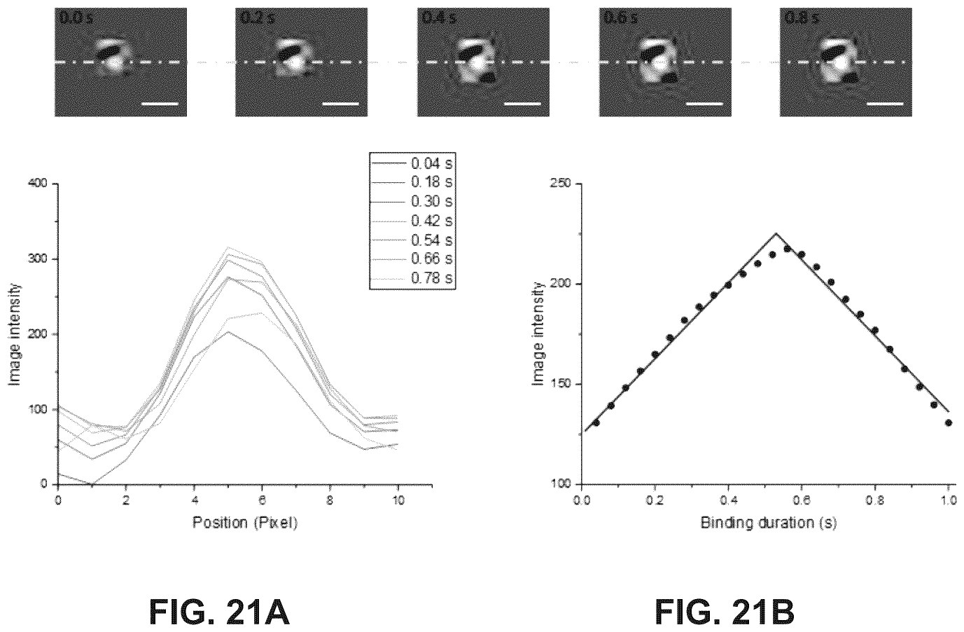

Ultra-sensitive quantitation was achieved by the optimized data analysis approach (FIG. 20). Frist, the noise in the image was reduced by a n frame rolling averaging in the raw video starting at frame m, Nₘ^{m+n}, and then normalizing each frame of the averaged video in terms of their mean pixel value to avoid the intensity change from the light source (as described further herein). Next, a differential image sequence (Nₘ₊ₙ^{m+2n}−Nₘ^{m+n}) was obtained from the normalized image sequence by subtracting each frame from the following frame, which removed static background and revealed binding or unbinding events. Second, a probability image (PI) sequence was obtained by convoluting the differential image sequence with Haar-like array, which would reveal the morphological characteristics of the particle. The candidate pixels were selected if its intensity is higher than the mean intensity+3×standard deviation (s.d.) of the whole PI. Third, a fixed region was extracted in the differential images at positions of the candidate pixels mapped from the PI and fitted with a 2D Gaussian model to obtain the precise center location and intensity of the particle. The candidate pixels with a fitting biased too much from a Gaussian blob would be deleted. Last, as the binding of a particle appears as a brightening Gaussian blob and then gradually disappears (FIG. 21), the intensity of the single particle was determined as the peak of its temporal intensity profile.

The relationship between molecular weight (MW) and image intensity of particles for PSM had been demonstrated previously. Here, several pure protein samples were measured to determine such a relationship on our setup (FIG. 5b). With this information, whether a bound single particle was a detection antibody was determined based on its image intensity. The binding events was considered nonspecific if they happened at the same location multiple times. The difference between specific and nonspecific binding is shown in the supporting video. These analyses are critical for removing confounding effects caused by complex sample matrices like blood and greatly improved the counting accuracy for specific single molecule binding events.

### Validation of Label-Free Assay by IL6 Detection

To assess the intrinsic sensitivity of LFSMiA, we first applied it to interleukin-6 (IL-6) detection, a key factor in hematopoiesis, immunomodulation and inflammation processes, in pure buffer (1×PBS containing 1% BSA) and bovine serum. A series of standard buffer solutions or spiked bovine serum samples with different concentrations of IL-6 were flowed through the microfluidic channel for 2 hours to react with the capture antibody pre-functionalized on the sensor surface. Next, 10 nM detection antibody was injected for 10 mins to bind with IL-6 captured by the surface antibody, which is monitored by PSM (FIG. 5a). The number of IL-6 caught on the sensor surface was tracked by counting the binding events of the detection antibody with a temporal resolution of ˜10 ms. The temporal profile of the binding events follows the association kinetics of binding and correlates with the concentration of IL-6 (FIGS. 5c and 5d).

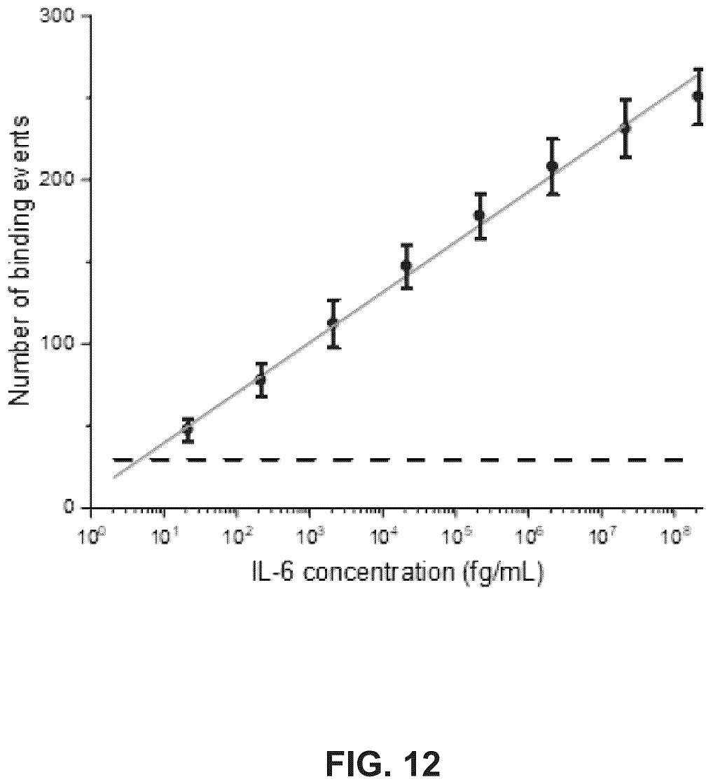

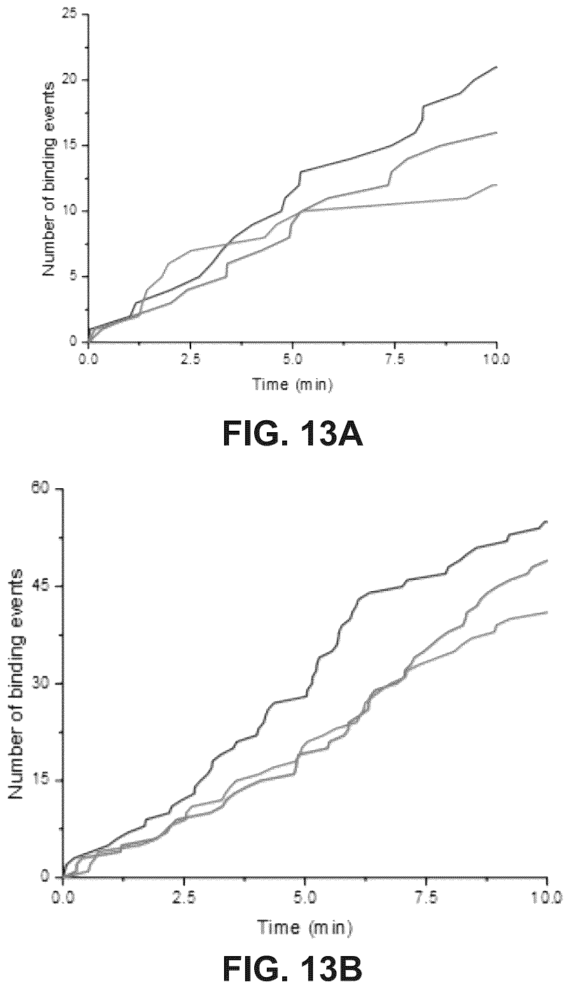

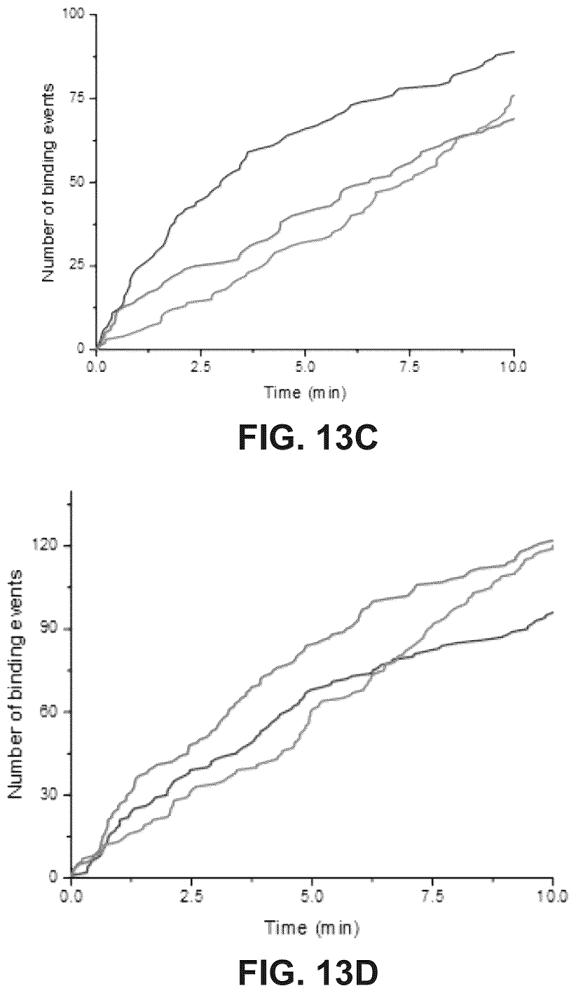

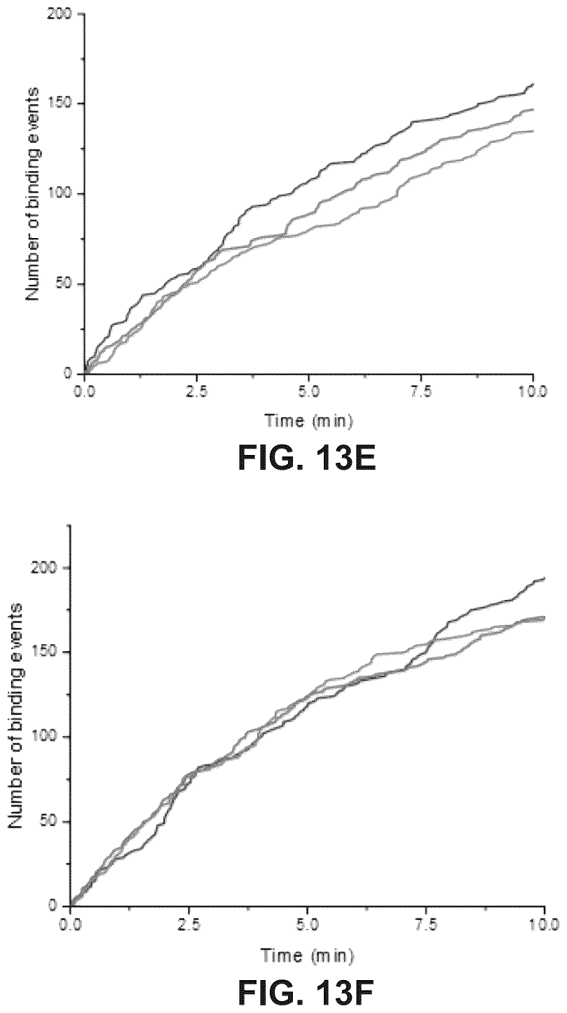

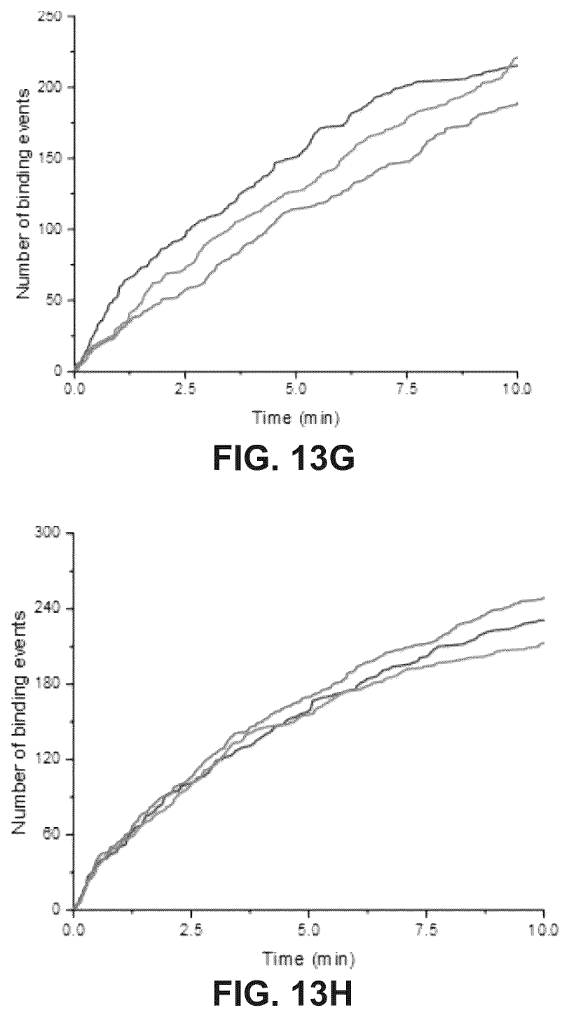

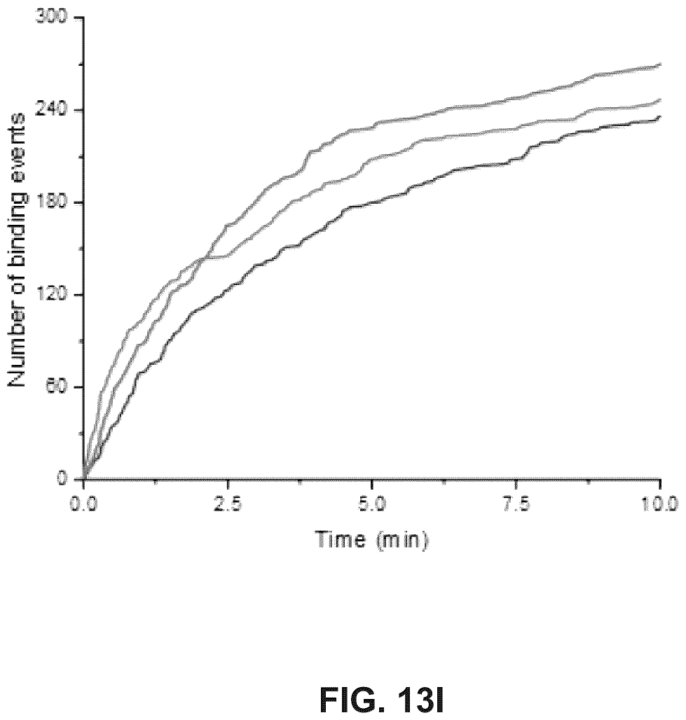

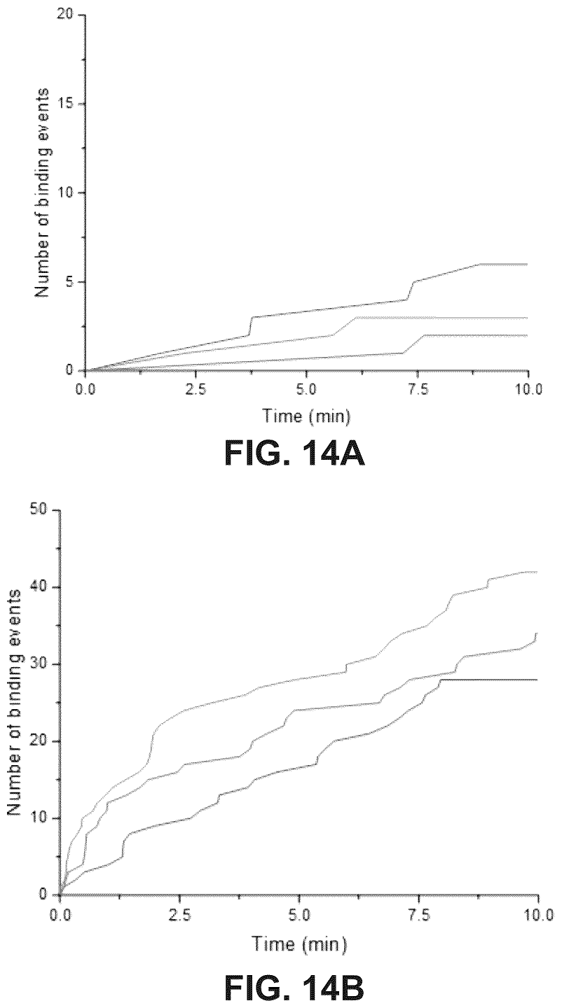

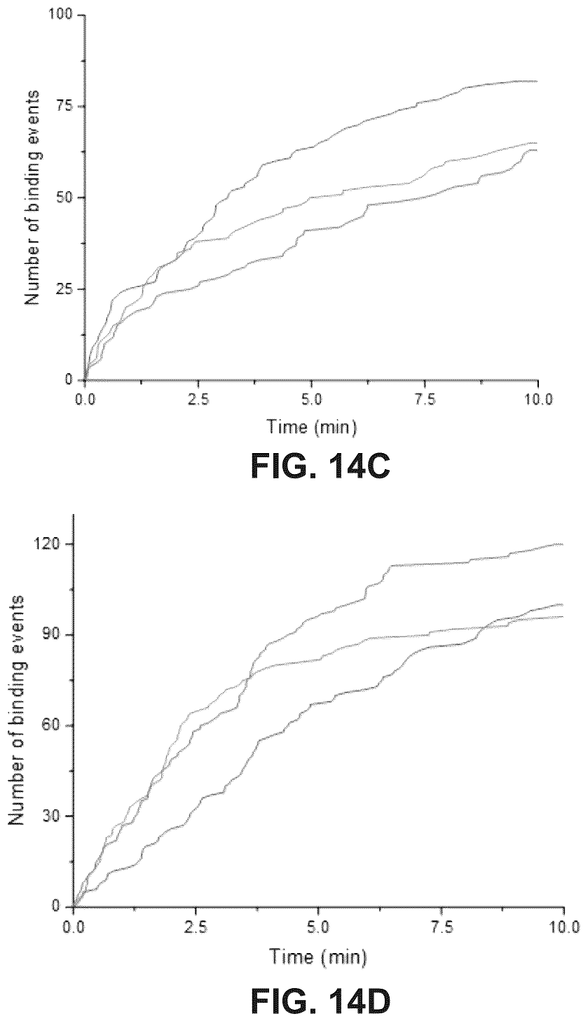

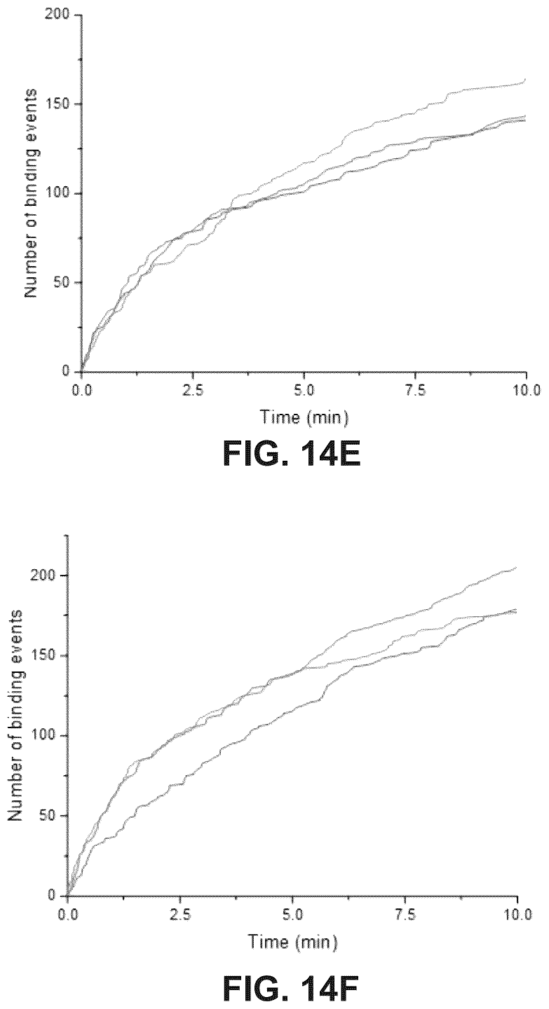

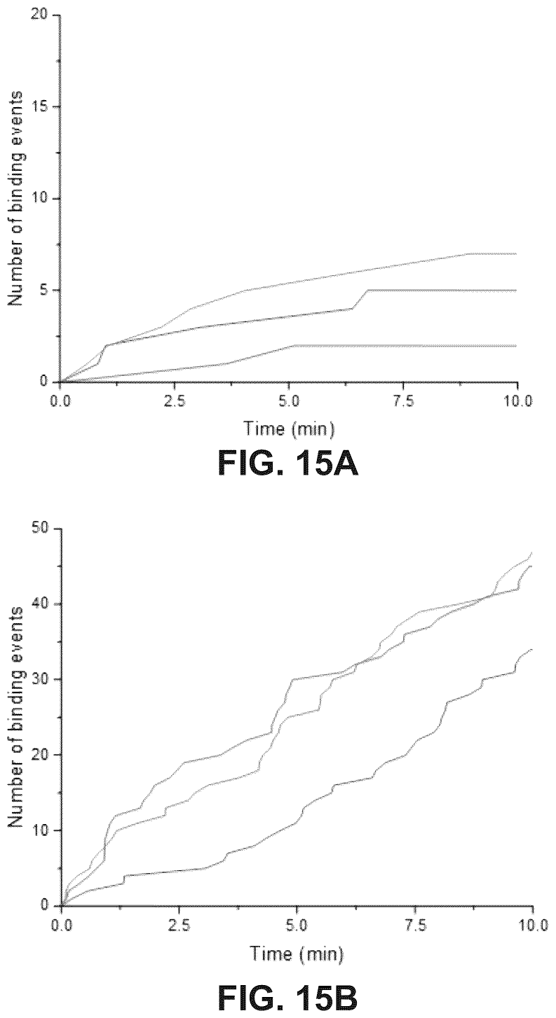

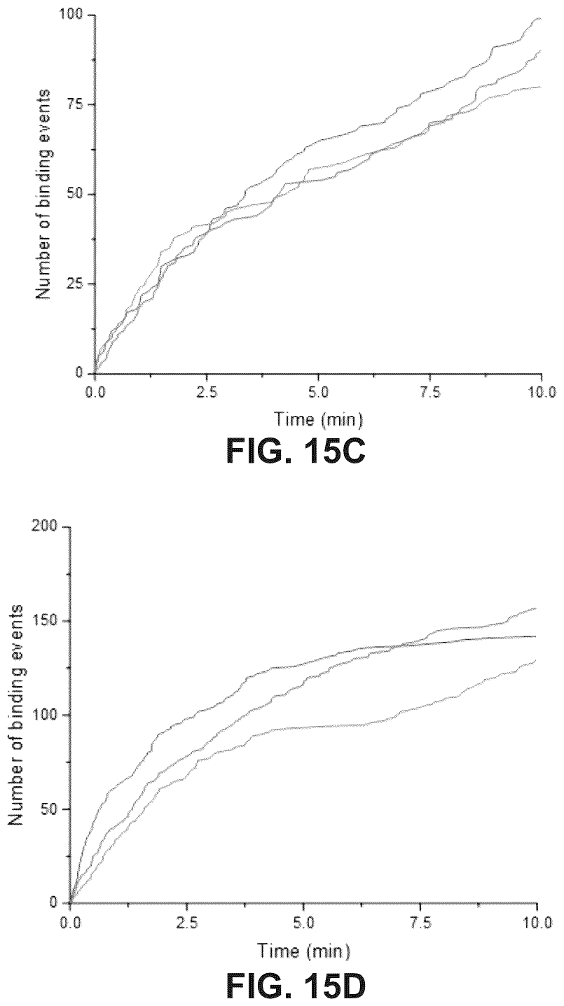

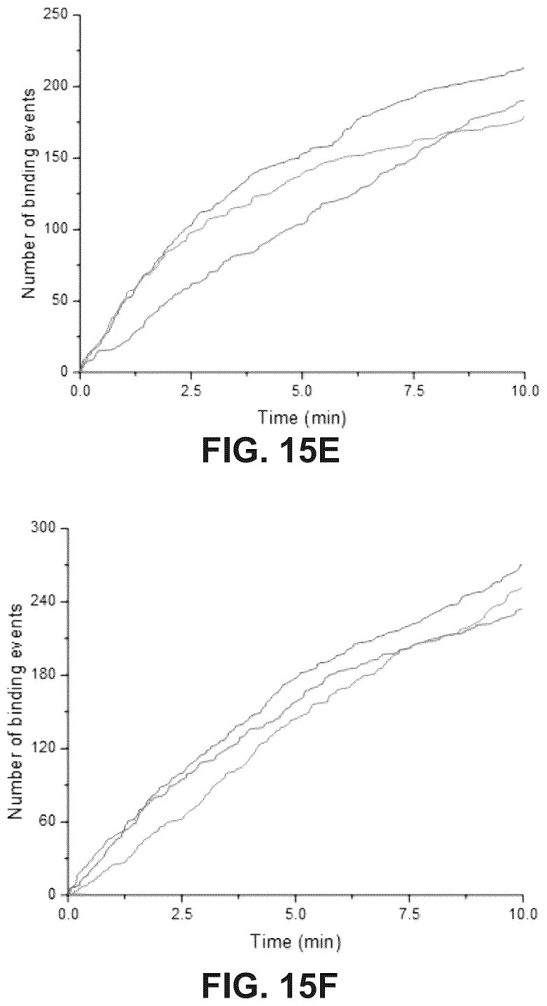

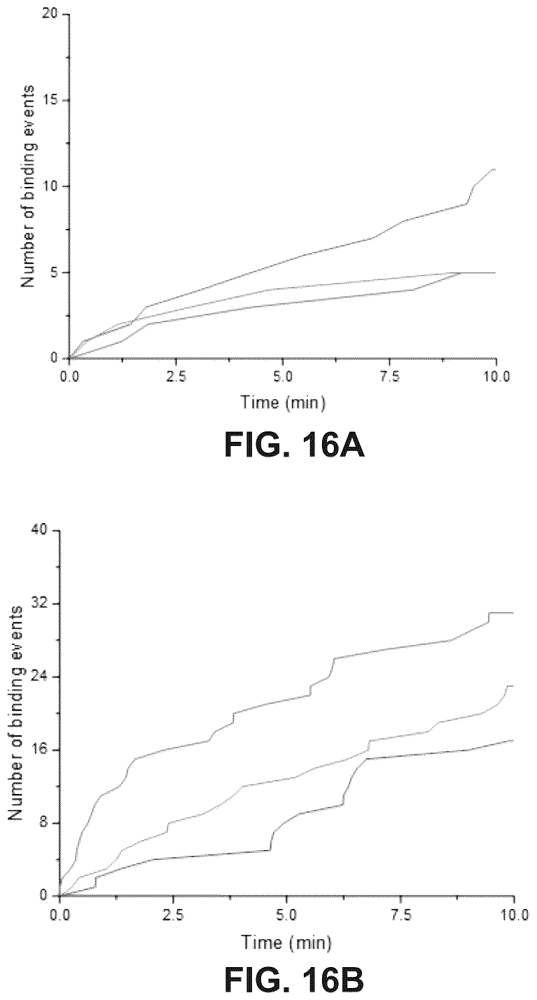

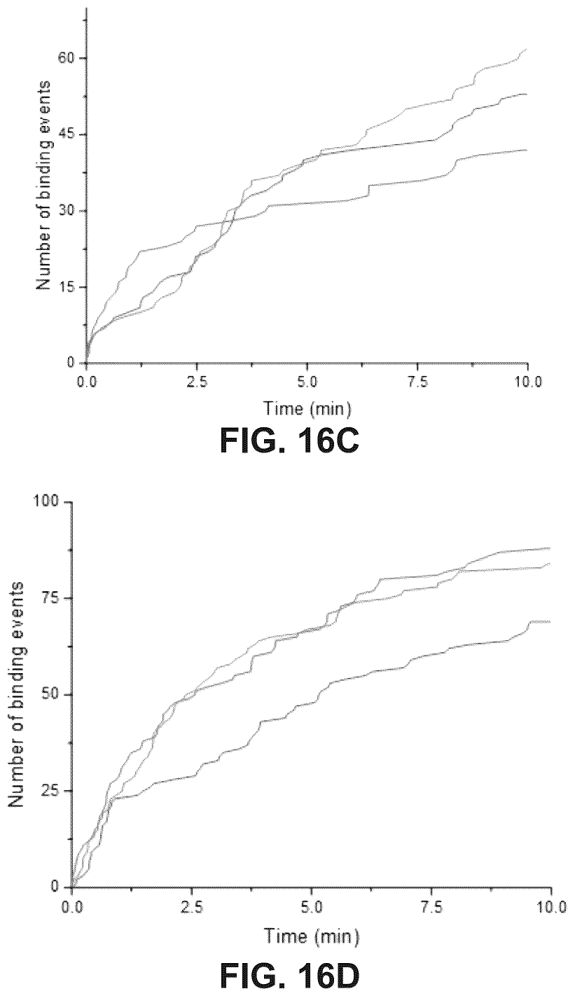

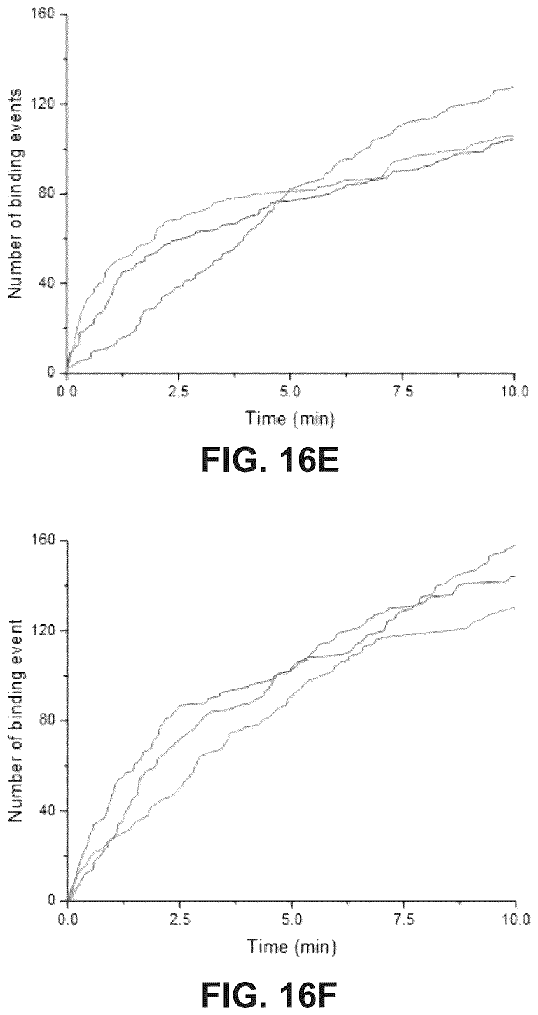

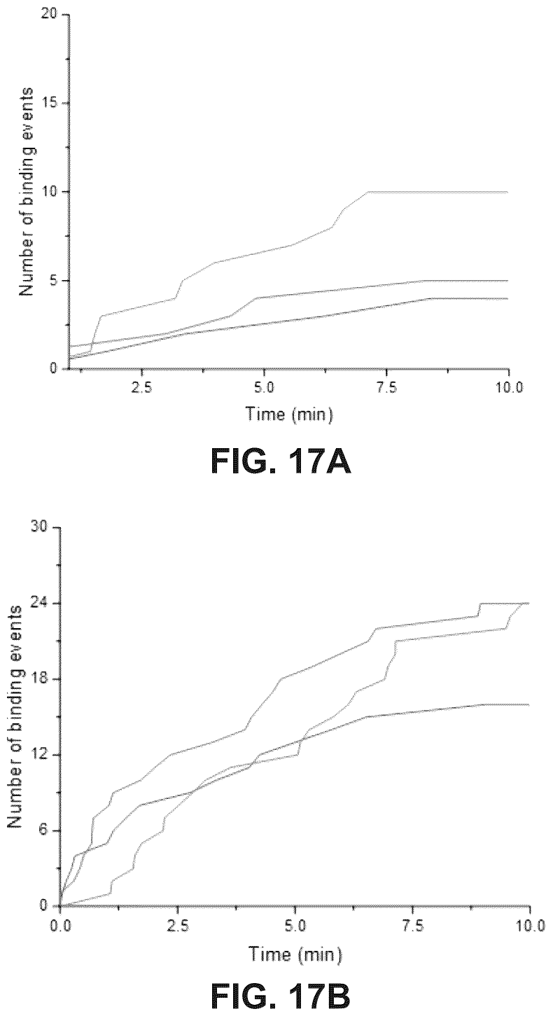

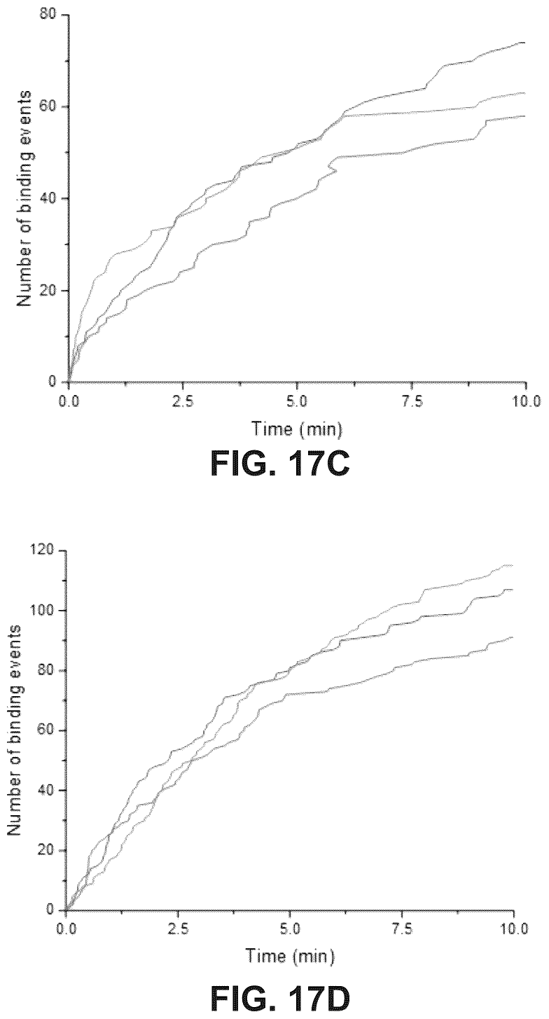

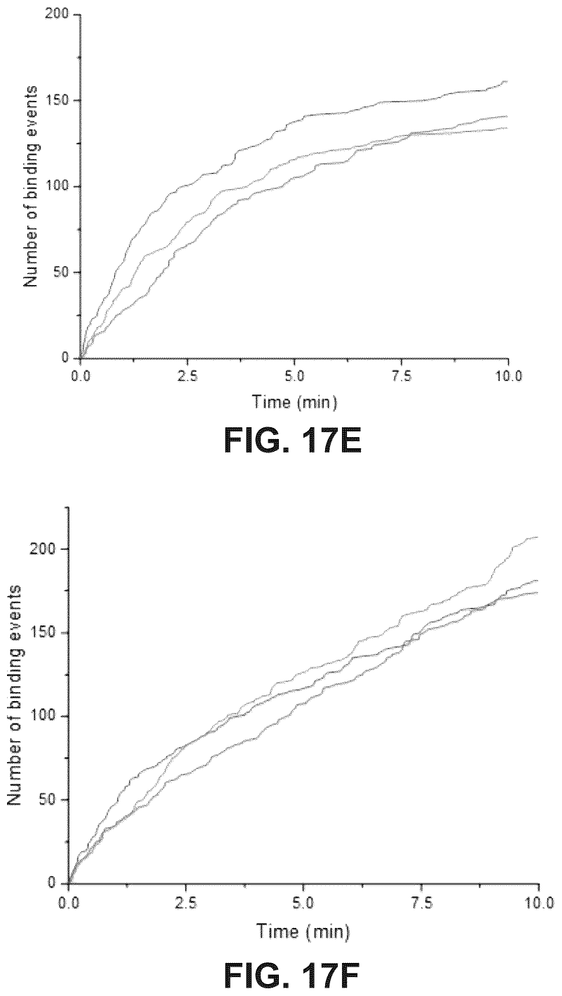

The standard curves of IL-6 detection in pure buffer and bovine serum were determined with 3 replicates for each concentration (FIG. 5e). Defined as the estimated counts of blank experiments plus three times of its corresponding s.d., the limit of detection (LOD) was calculated to be 1.89 fg/mL and 3.91 fg/mL (0.09 fM and 0.19 fM) in pure buffer and bovine serum, respectively. The level of nonspecific binding event in bovine serum was lower than in pure buffer, likely because bovine serum blocking the sensor surface better. For clarity, only part of data was shown in FIGS. 5c, 5d and 5e and complete data could be found in the supporting information. We have realized an eight log detectable dynamic range, from 1.89 fg/mL to 0.21 μg/mL (FIG. 12). The sub-femtomolar sensitivity and high dynamic range of the label-free assay results from two factors: (i) most of the nonspecific binding could be removed based on the intensity, duration and morphology of the binding events, and (ii) dynamic tracking of single particle binding is less limited by spatial optical resolution and capable of detecting binding signal even when surface is crowded by particles.

### Rapid NT-proBNP Detection Under Clinical Setting

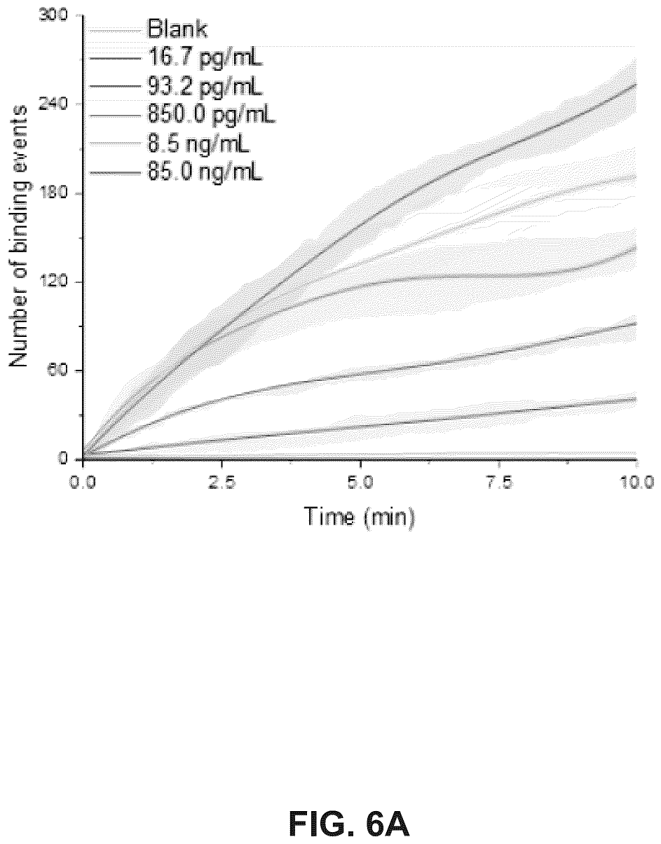

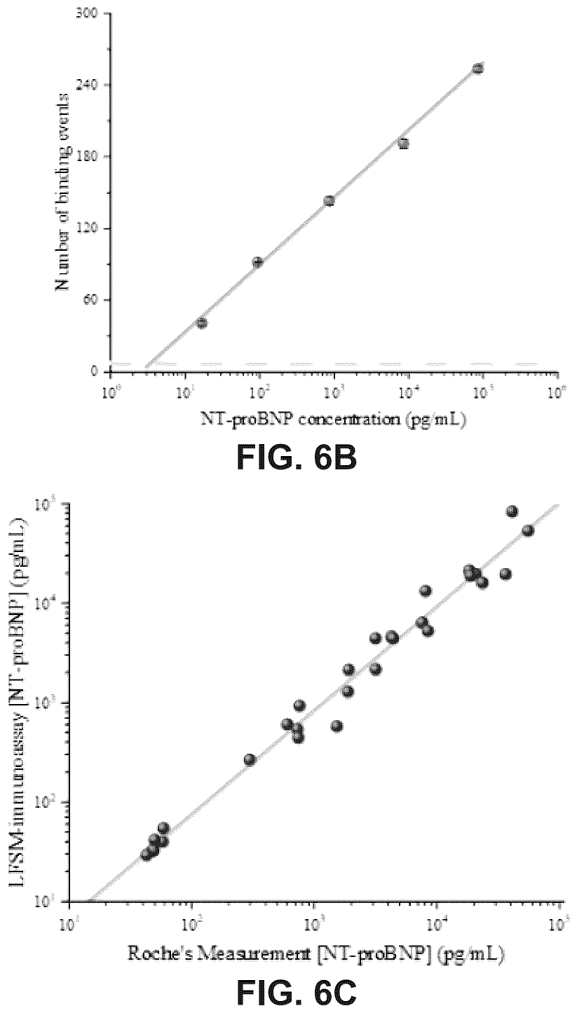

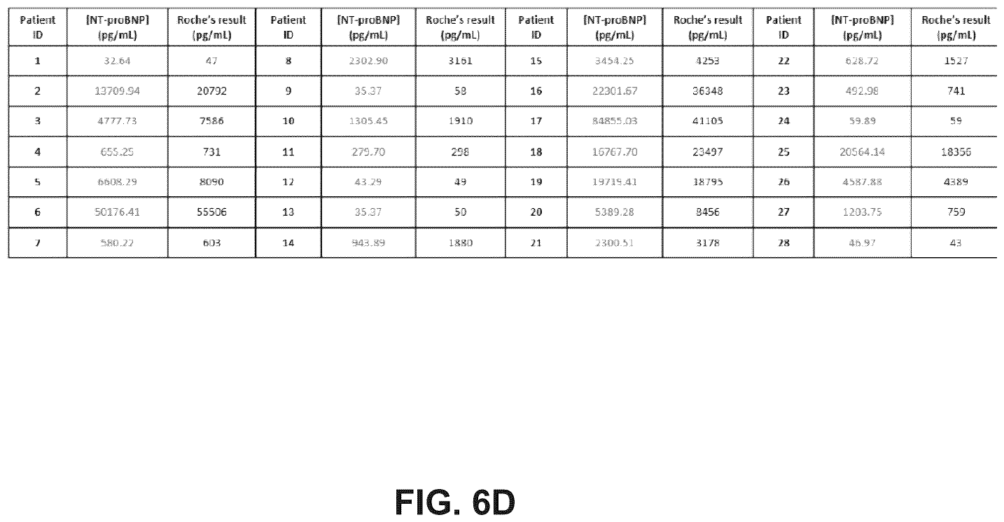

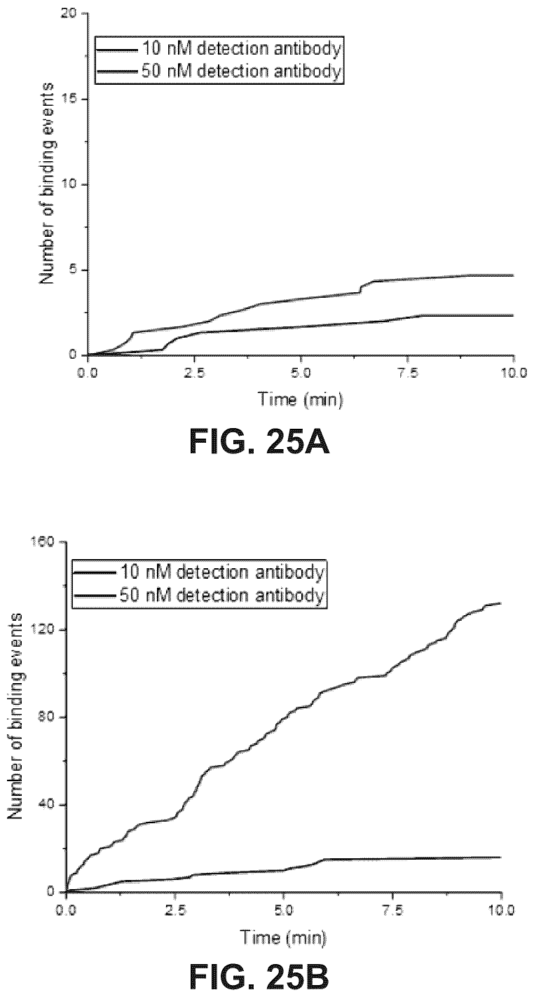

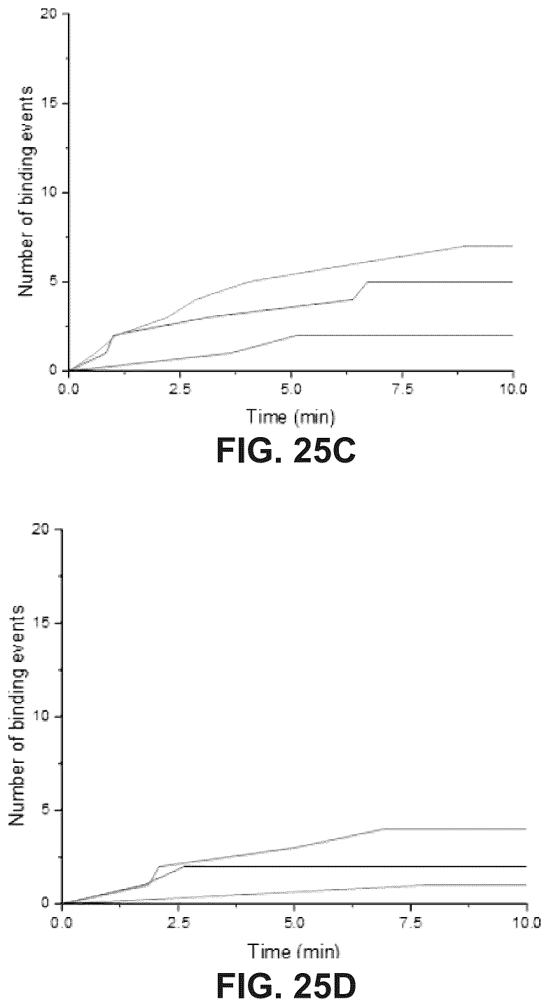

Simple, rapid and sensitive assays are always needed in clinical setting. To show the applicability of our assay for clinical use, it was evaluated for NT-proBNP detection, a key clinical biomarker of heart failure. As NT-proBNP naturally presents in human blood circulation, we first measured the baseline level of NT-proBNP in the human plasma pool used for spiking, which was determined to be 8.23 pg/mL. A series of standard samples with different concentrations of NT-proBNP were prepared by spiking recombinant NT-proBNP into the human plasma pool with baseline level corrected. To evaluate the background level, horse serum was used as blank control without human NT-proBNP. Similar to IL-6 experiment, these standard samples were introduced into the microfluidic channel for 10 mins (FIG. 5a). Then, 50 nM detection antibody was injected for 10 mins. 50 nM was selected as it could amplify the sensor response but keep the nonspecific binding low enough (FIG. 25). The same PSM setup and parameters as IL-6 detection were used. The temporal profiles of the binding event were shown in FIG. 6a. After repeating the experiments 3 times, the standard curves of NT-proBNP detection in human plasma were determined (FIG. 6b). The LOD was determined to be 3.39 pg/mL.

To evaluate the translational potential of LFSMiA for diagnostic applications, a cross-validation between the Roche's Elecsys proBNP II assay, an FDA approved routine clinical diagnostic testing for heart failure, was conducted. Serum samples from 28 patients were measured using both the Elecsys proBNP II assay and LFSMiA. The measured concentration of our method was strongly correlated with those of the Elecsys proBNP II assay (FIG. 6c, slope=1.04 and R>0.99), which indicates a high potential of our method for POC application. More details can be found in Table 1. Overall, we have realized sub-picomolar detection with a 20-min total assay time. The dynamic range and detection limit of LFSMiA also satisfies the requirement for clinical NT-proBNP testing.

### Protein Detection in Whole Blood

As modern medicine greatly depends on clinical testing for disease diagnosis and monitoring, a simple assay for direct analysis of whole blood components is always preferred. Detection method using whole blood as sample matrix could not only reduce the loss of biomarkers caused by physiolysis and manual operation, but also minimize total assay time by eliminating sample preparation steps. Recent single-molecule technologies, like single molecule enzyme-linked immunosorbent assay and single molecule fluorescence microscopy, either requires removal of blood cells or have to dilute whole blood to achieve reliable detection due to matrix effects or extreme background autofluorescence. Biomarker detection at single molecule level in undiluted whole blood was accomplished by our method owes to three important features: 1) The use of microfluidic flow helps to reduce blocking effect caused by red blood cells. 2) The label-free nature, together with the two-step assay format, dramatically lowers the background signal by separating signal readout from biomarker surface capture in whole blood, enabling reliable detection of single binding events. 3) Real-time PSM imaging removes nonspecific background signals and reduces matrix effects.

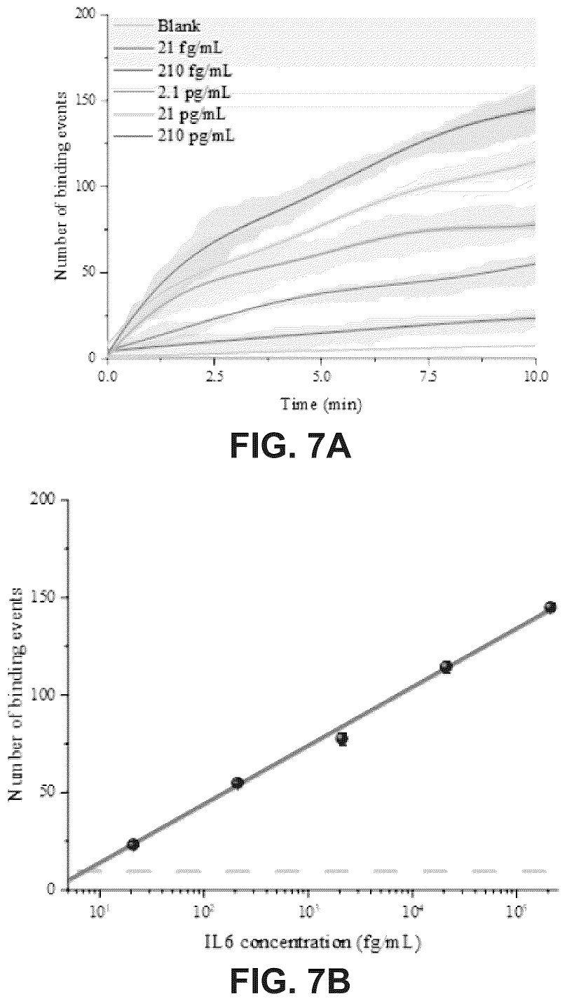

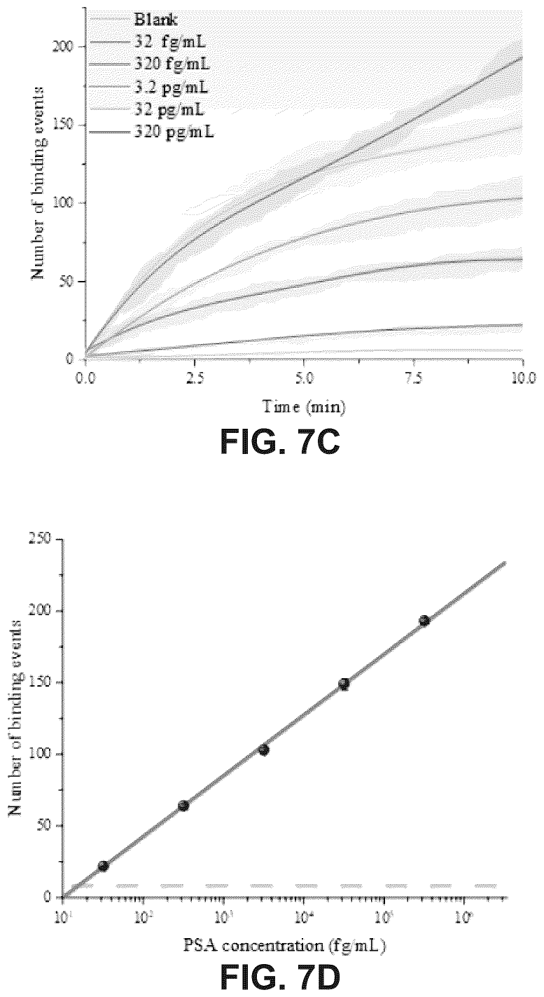

To validate the capability of this method for direct detection in whole blood, we conducted experiments using the label-free digital immunoassay for the detection of interleukin-6 (IL-6) and prostate-specific antigen (PSA). Bovine whole blood was spiked with varying concentrations of IL-6 and PSA, and these samples were analyzed using the label-free digital immunoassay, following the same protocol used in IL-6 detection. The temporal profiles for IL-6 and PSA detection are shown in FIGS. 7a and 7c, respectively. Standard curves for both biomarkers were generated after performing the experiments in triplicate (FIGS. 7b and 7d). The limit of detection (LOD) for PSA in whole blood was determined to be 16.27 fg/mL (0.51 fM), while that for IL-6 was 7.36 fg/mL (0.35 fM). As expected, the slopes of the two standard curves differed due to the variance in affinity between the respective antibody pairs.

These results confirm the feasibility and robust performance of the LFSMiA for ultra-sensitive biomarker detection. To our knowledge, this is the first demonstration of rapid, digital protein detection using label-free single-molecule technology. Our method exhibits sensitivity comparable to other single-molecule assays, with the added advantage of detecting individual biomarkers directly in undiluted complex clinical fluids such as serum, plasma, and whole blood—a capability not previously reported in single-molecule technologies.

### Point-of-Care LFSMiA

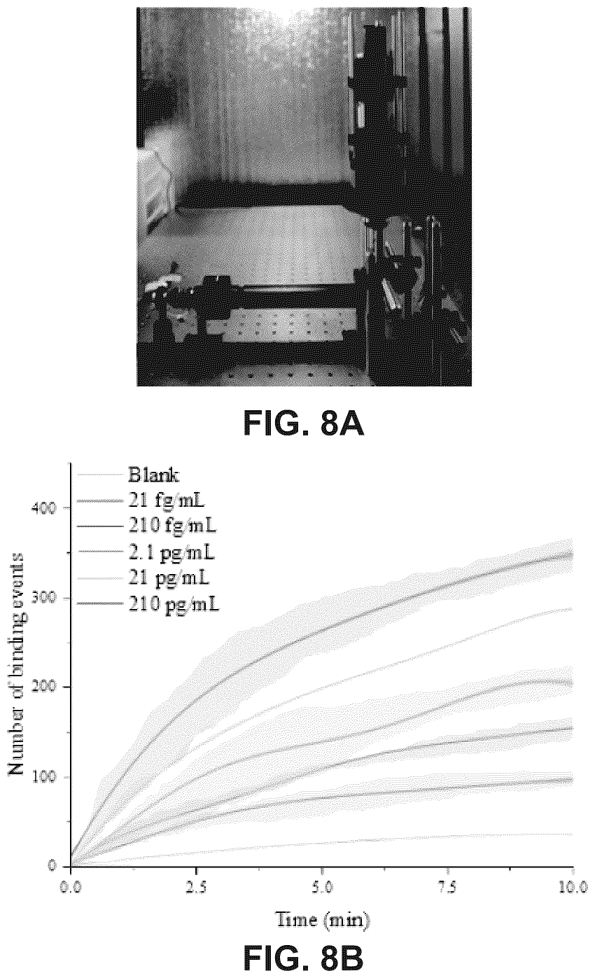

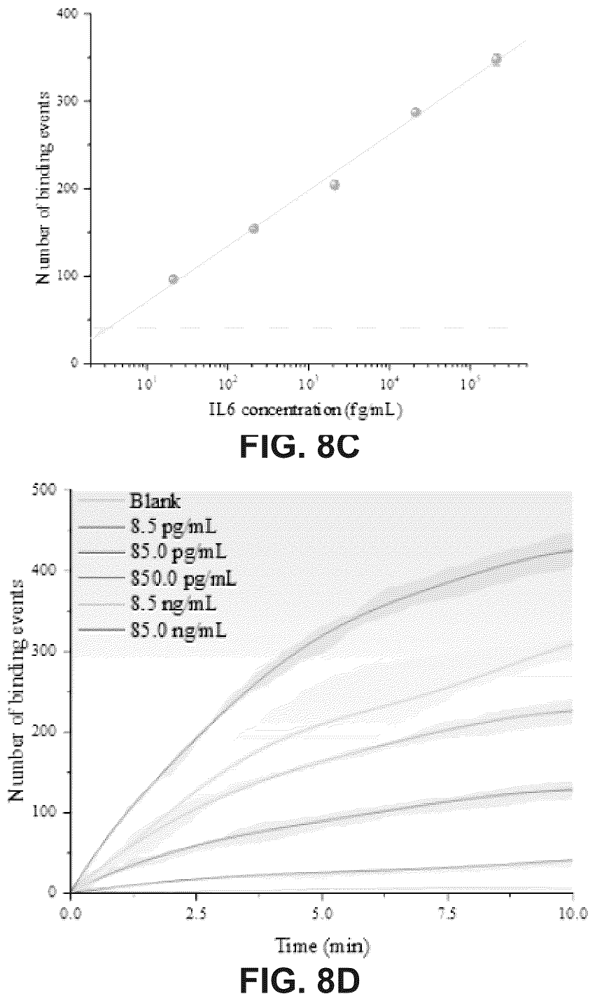

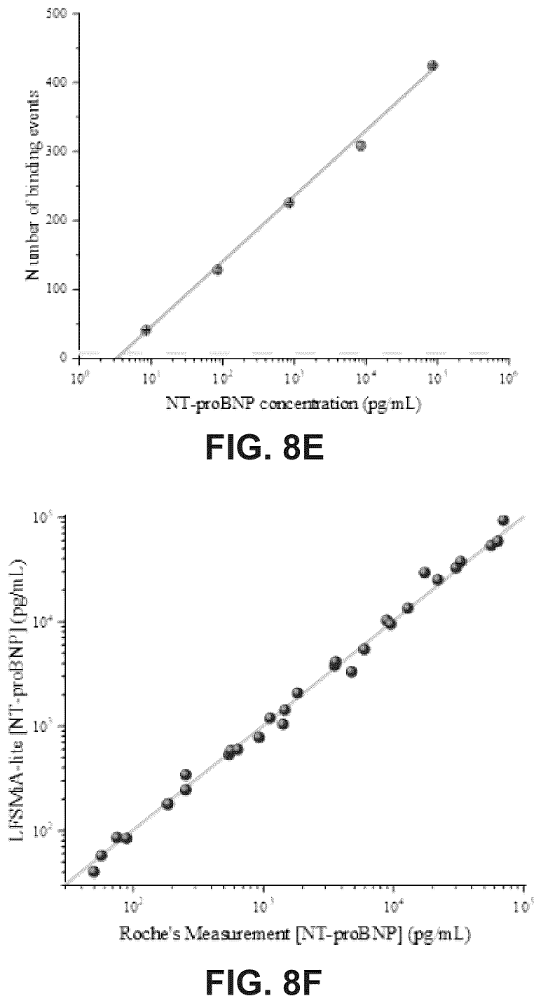

To assess the applicability of the LFSMiA in point-of-care (POC) settings, we developed an affordable label-free single-molecule microscope (LFSMiA-lite) with compact dimensions of 40×15×40 cm, constructed using commercially available optical components. The total cost of the setup is approximately USD 2,000 (FIG. 8a). Furthermore, we expanded the imaging area to achieve higher particle counts within 10 minutes, thereby enhancing the precision of the assay. To our knowledge, no commercially available microscopy system combines low cost, small size, and single-molecule sensitivity in a label-free format.

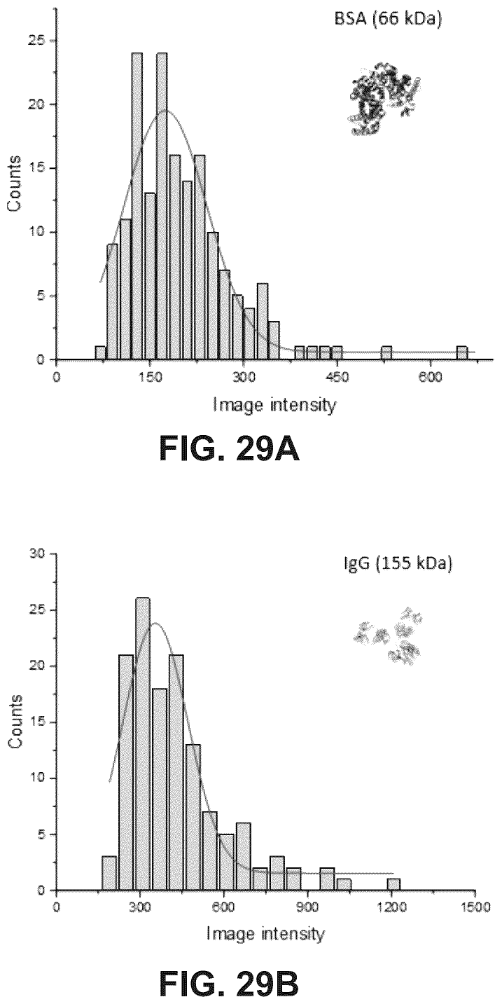

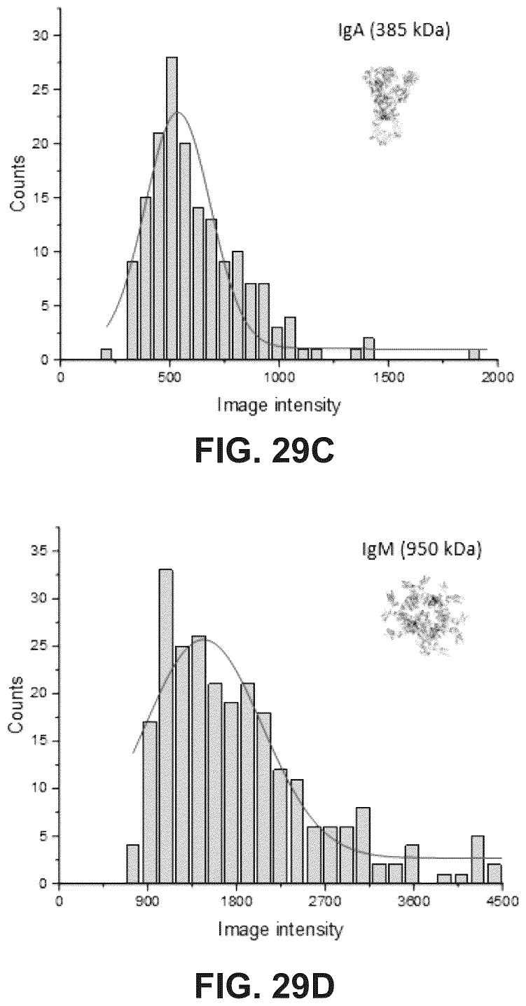

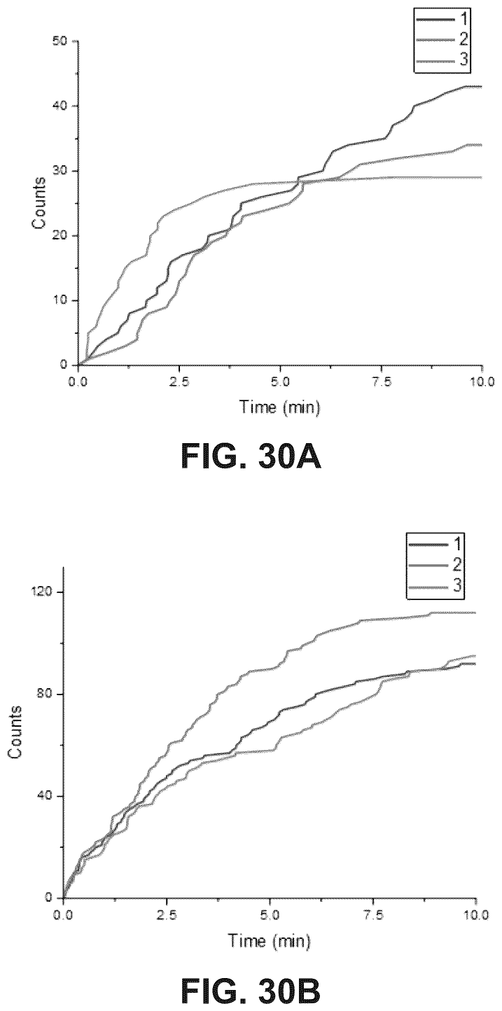

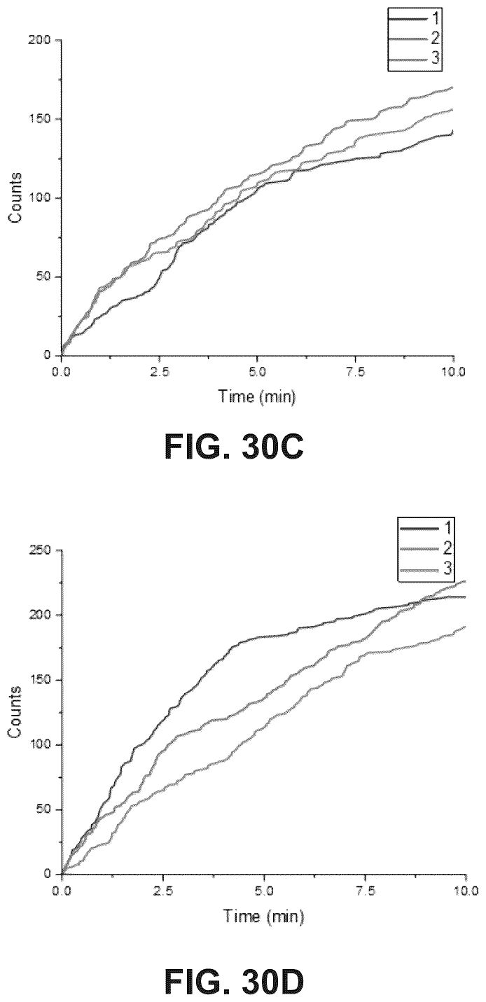

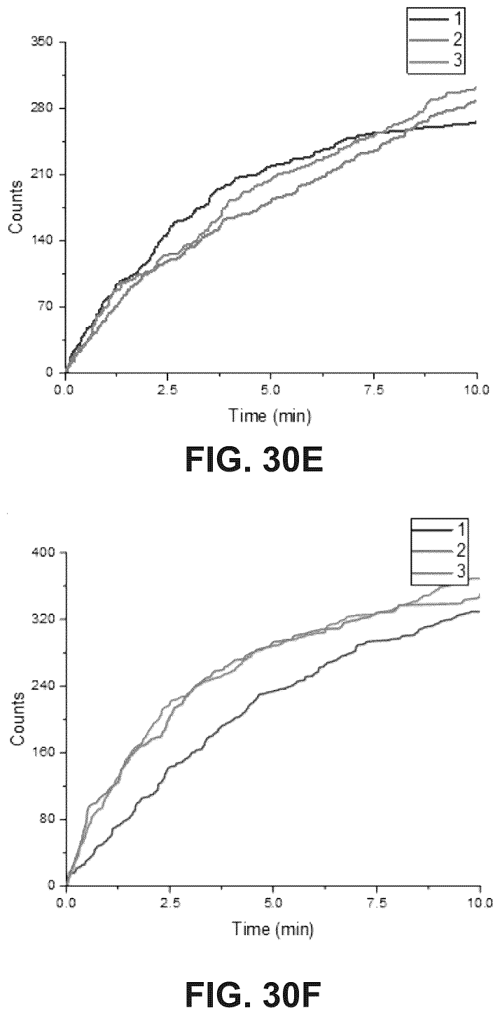

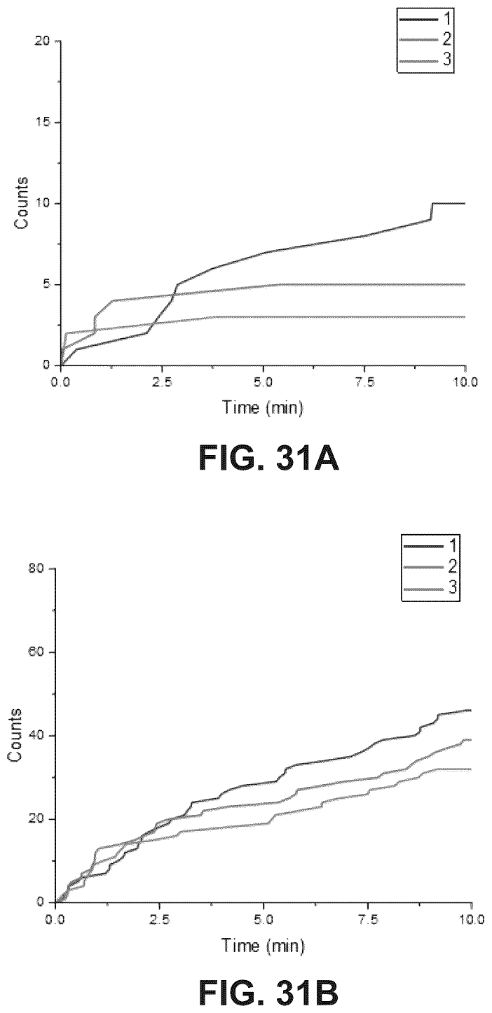

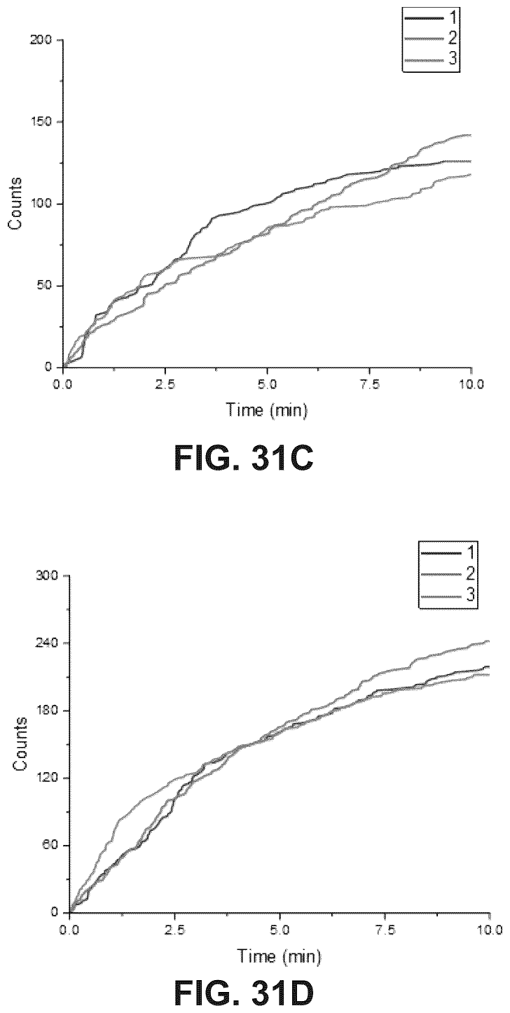

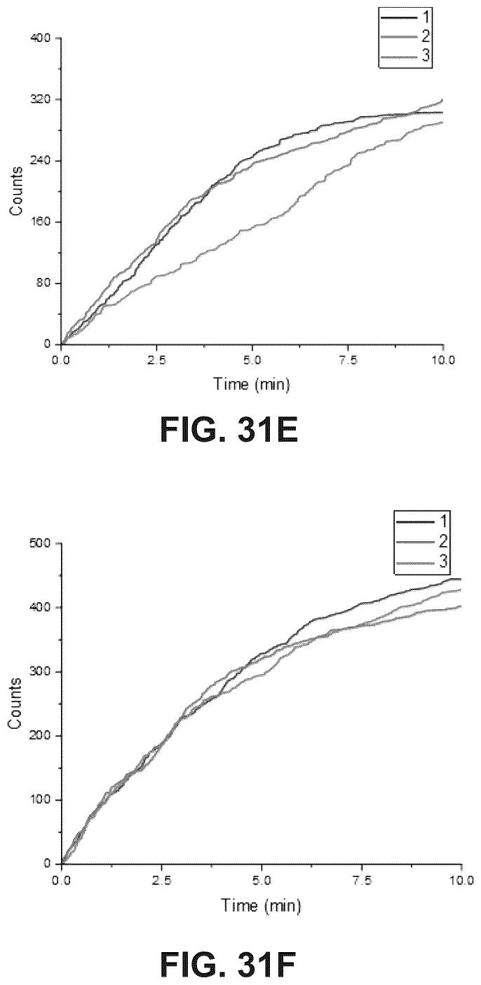

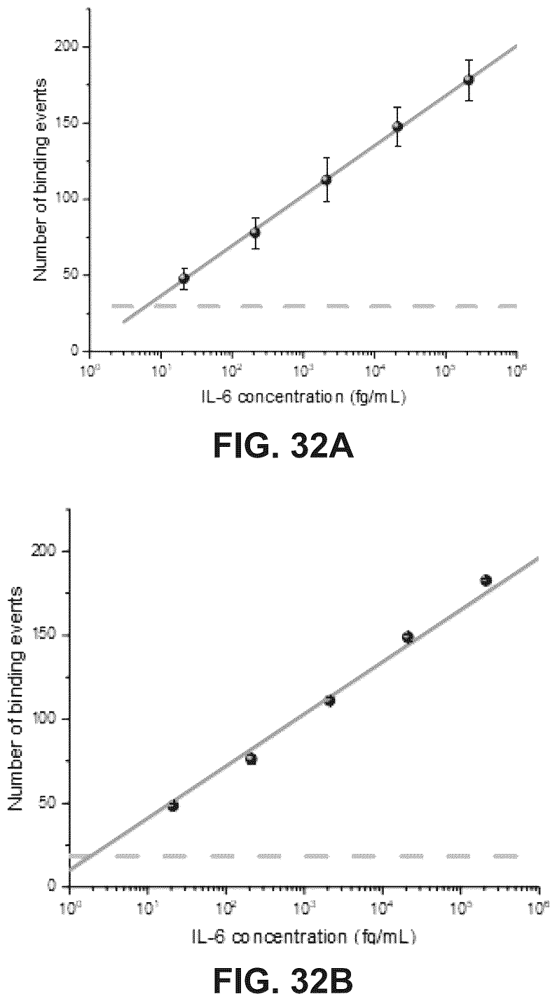

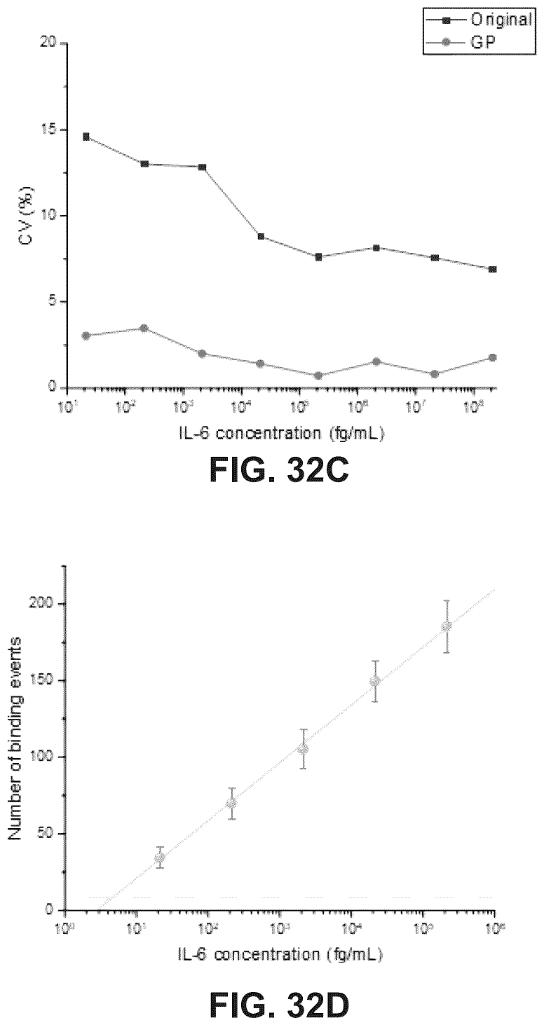

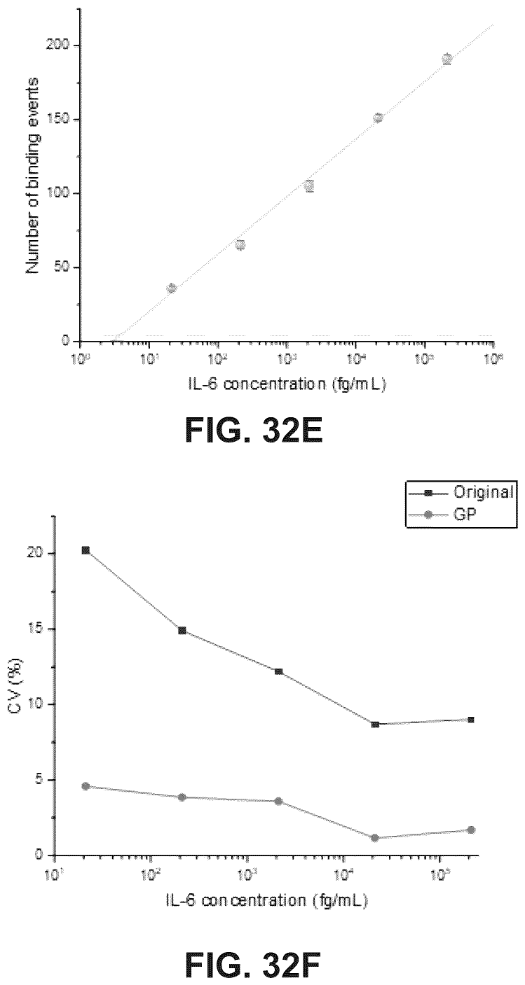

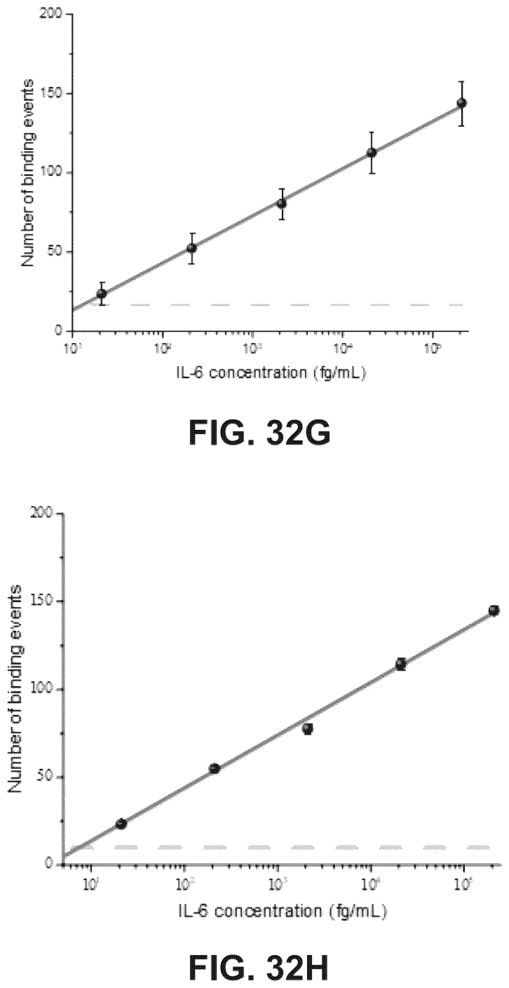

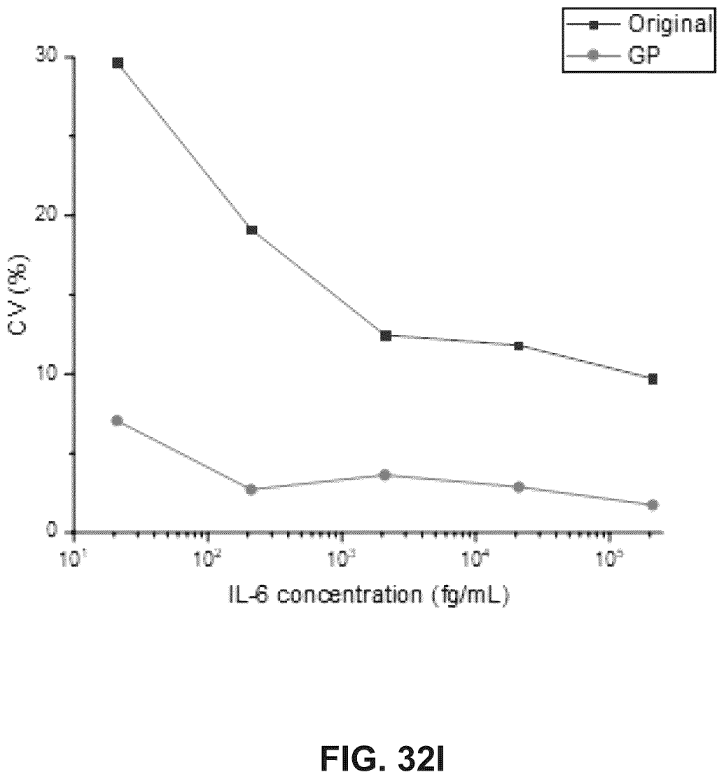

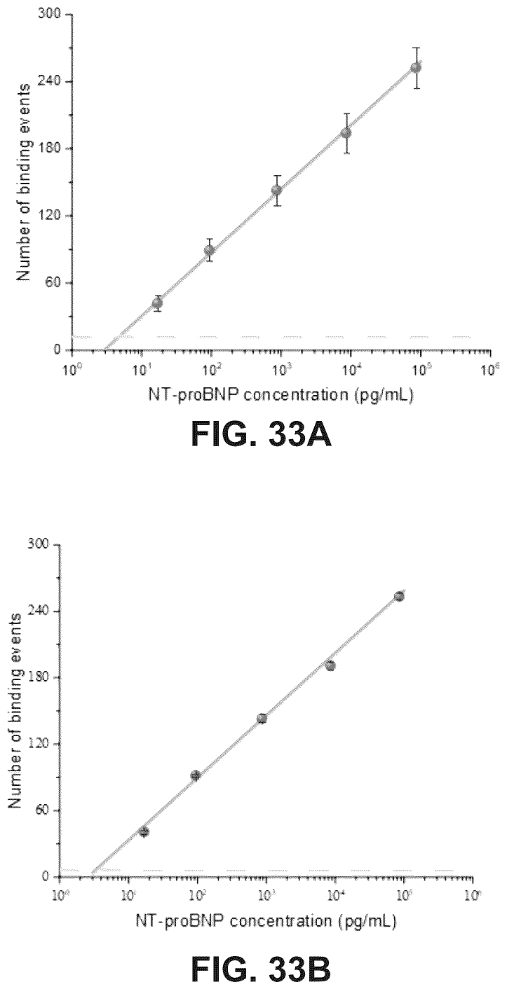

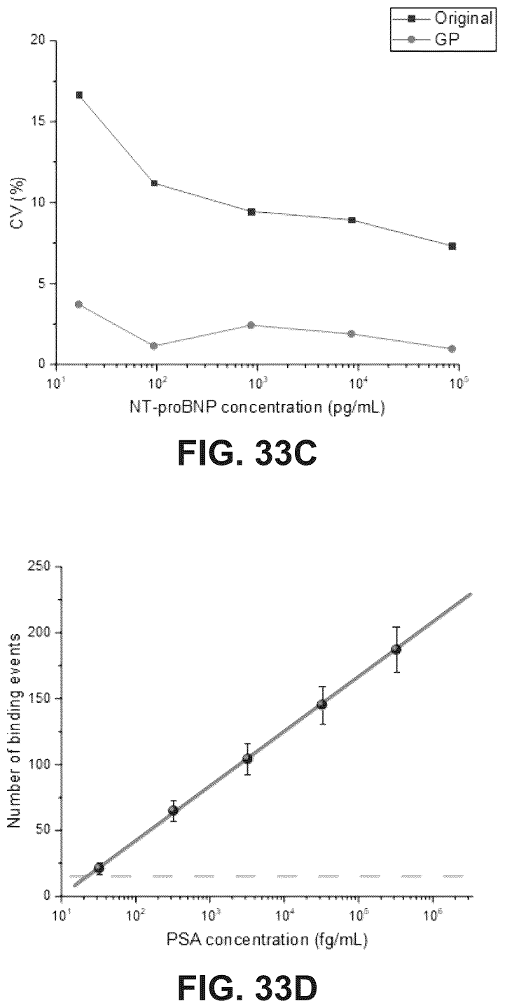

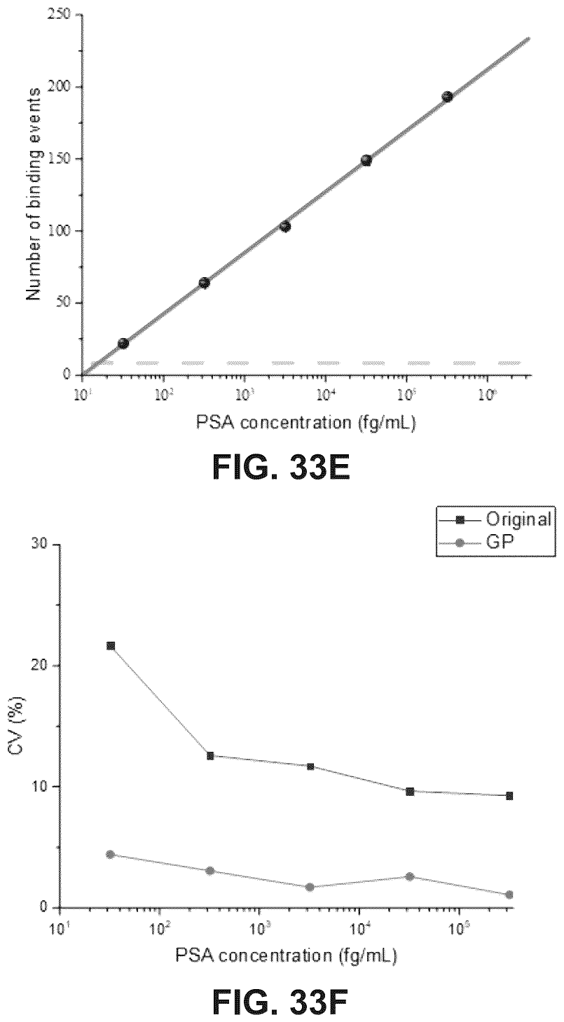

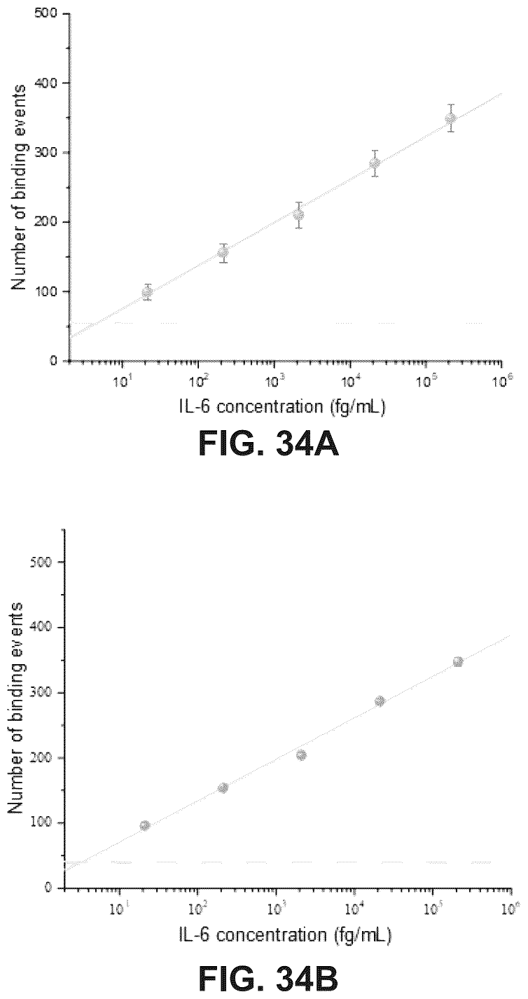

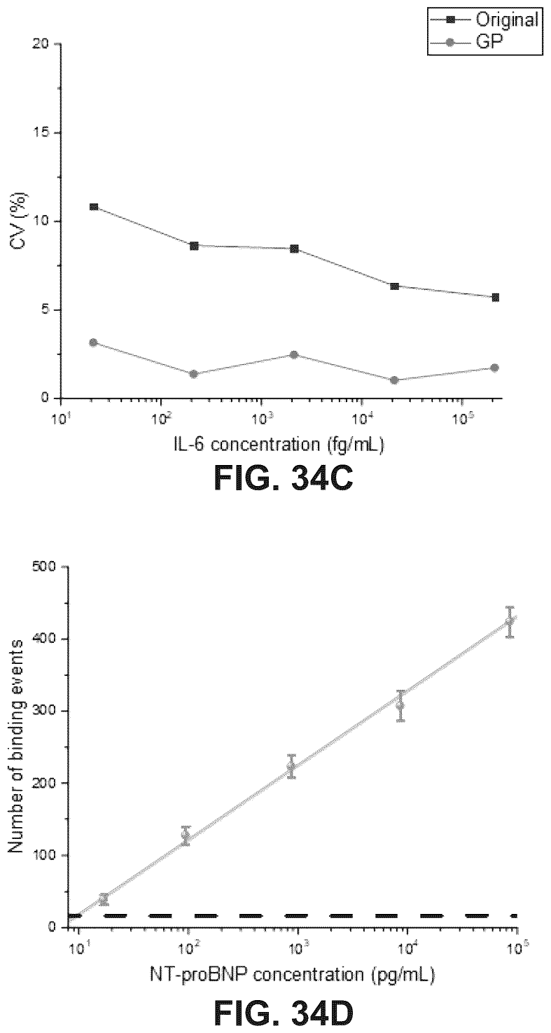

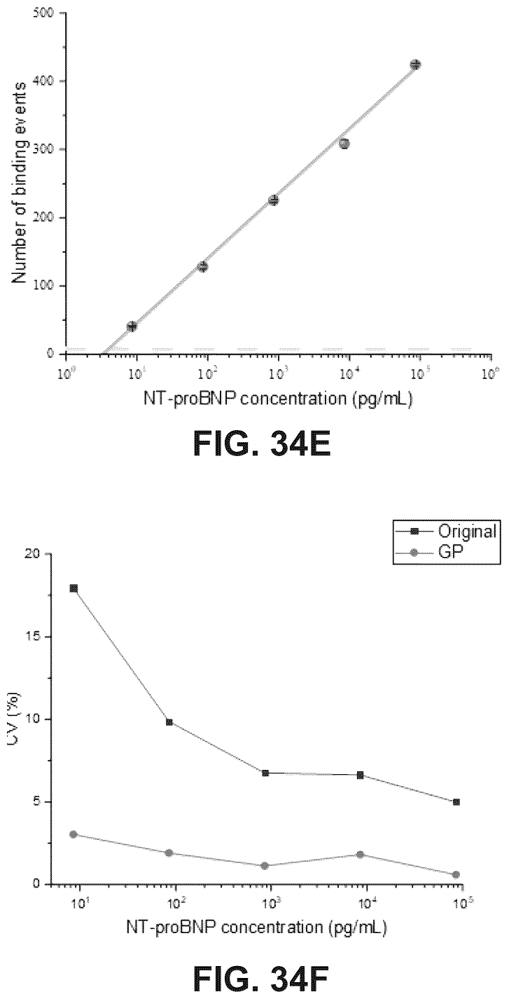

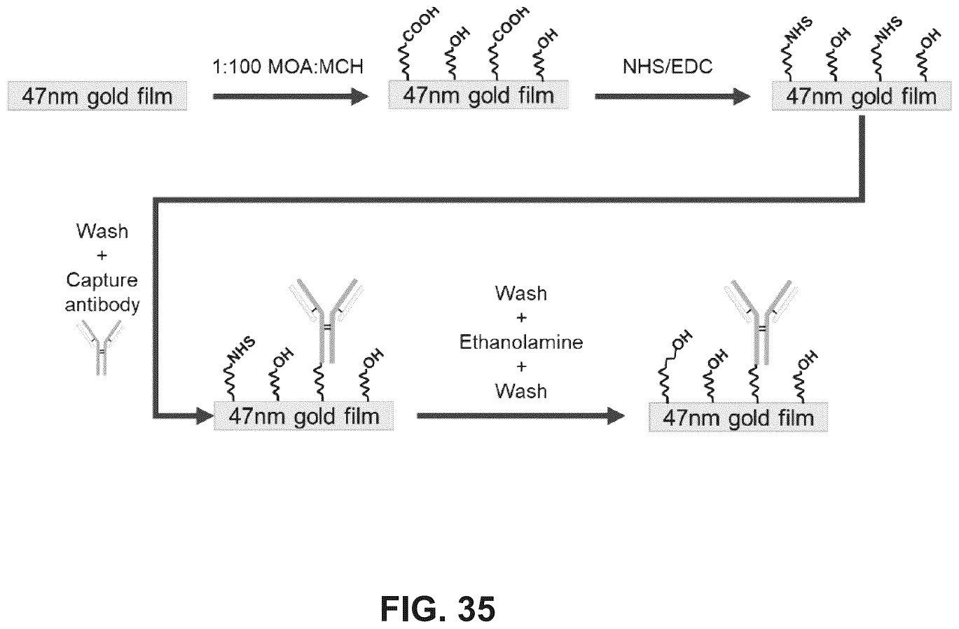

First, we established the relationship between molecular weight and particle image intensity using the new LFSMiA-lite system, enabling the determination of threshold values for optimized data analysis (FIG. 29). To compare the performance of LFSMiA-lite with our laboratory-grade microscope, we selected IL-6 as a model analyte. Following the same experimental protocol as the laboratory setup, LFSMiA-lite demonstrated a similar limit of detection (LOD), which was calculated to be 3.37 fg/mL (FIG. 8c). These results suggest that the sensitivity and quantitative detection capability of LFSMiA-lite remain uncompromised despite its smaller size and lower cost.

To demonstrate the clinical translational potential and POC applicability of LFSMiA-lite, we employed it for rapid NT-proBNP detection in whole blood. A series of bovine whole blood samples spiked with varying concentrations of NT-proBNP were analyzed using LFSMiA-lite, following the same procedure used in previous NT-proBNP detection experiments. The resulting temporal profiles are shown in FIG. 8d. After conducting the experiments in triplicate, a standard curve was generated, and the LOD was determined to be 4.14 pg/mL (FIG. 8e). Additionally, whole blood samples from 28 patients were measured using LFSMiA-lite, while the corresponding serum samples were analyzed with Roche's Elecsys proBNP II assay for cross-validation. The results obtained with LFSMiA-lite exhibited excellent correlation with the FDA-approved assay, indicating the strong potential of this method for blood biomarker diagnostics in POC settings (FIG. 8f).

### Improvements of Gaussian Process Model

Recent advancements in digital immunoassay techniques have primarily focused on endpoint detection, relying on the counting of final single-molecule events. The inherent relative counting error is fundamentally limited by \sqrt{N} (where N represents the total counts), which defines the digital noise constraint in such assays. One of the most significant distinctions between LFSMiA and other assays is that LFSMiA operates as a real-time assay. By fully utilizing both kinetic and statistical information, LFSMiA effectively surpasses the digital noise limitation inherent in traditional digital immunoassays.

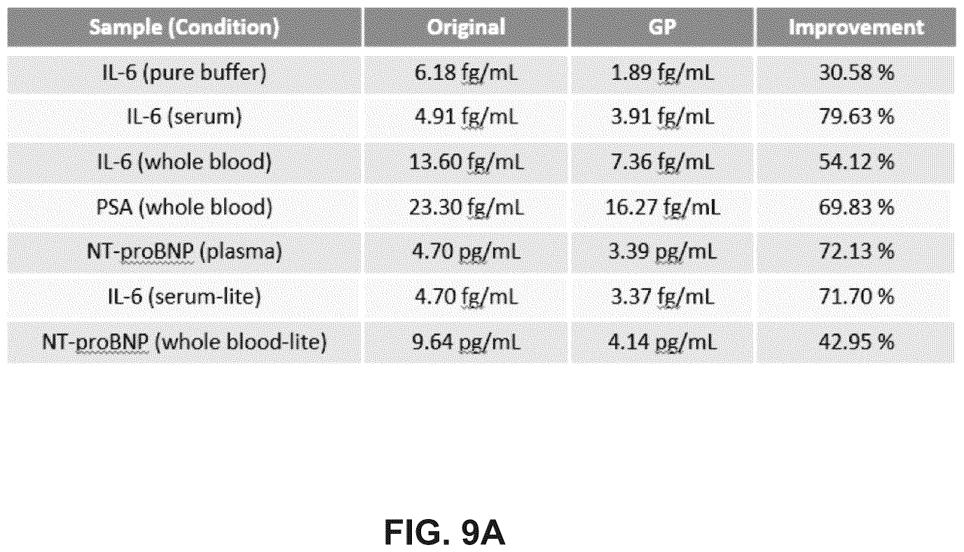

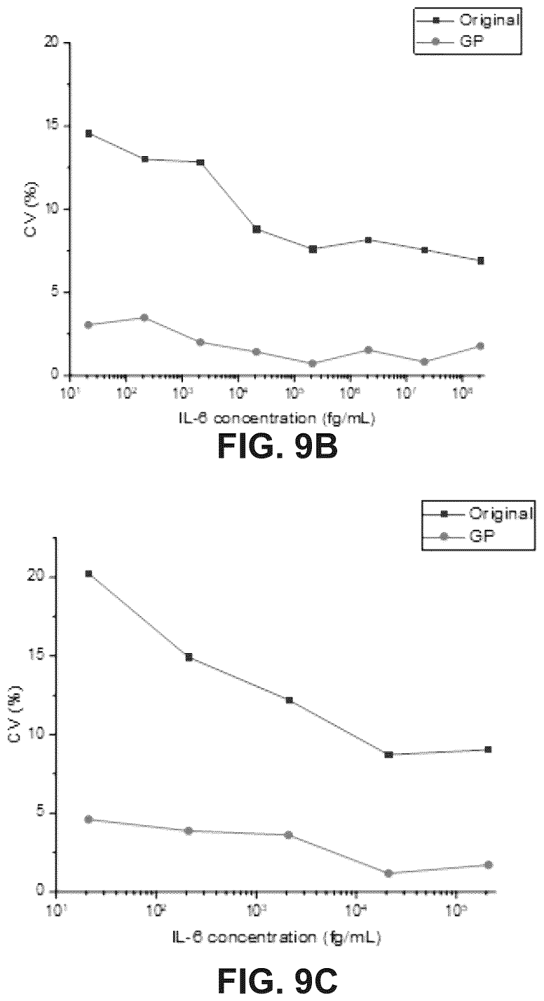

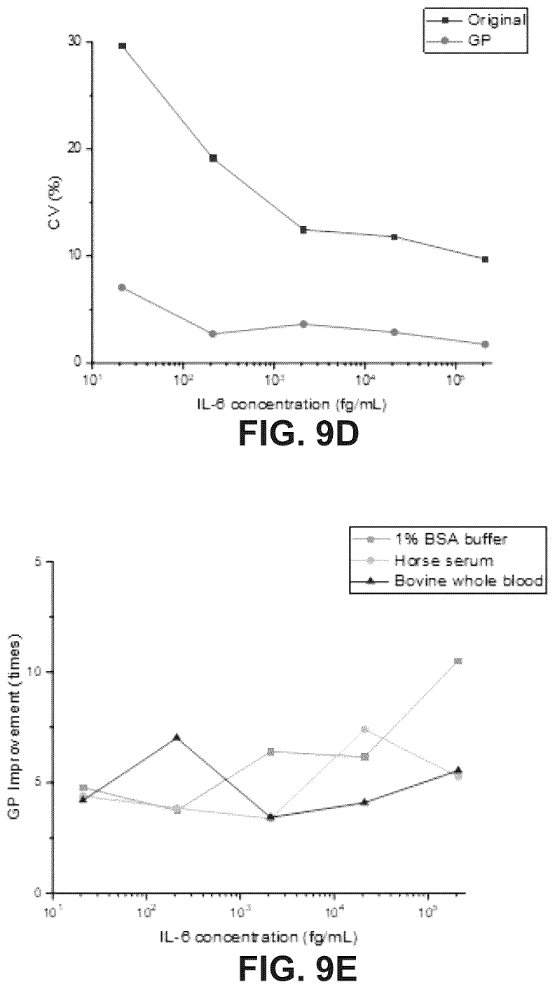

To demonstrate the improvements brought by the Gaussian process model, we first compared the limit of detection (LOD) with and without the use of the model, as illustrated in FIG. 9a. The Gaussian process model successfully reduced the original detection limit to 60.13% of its initial value. Subsequently, we evaluated the model's impact on the coefficient of variation (CV), which reflects detection precision. Improvements in the CV for IL-6 detection in pure buffer (1% BSA solution), horse serum, and whole blood are shown in FIGS. 9b, 9c, and 9d, respectively. The magnitude of improvement was also calculated and is presented in FIG. 9e. Notably, the Gaussian process model exhibits greater improvement at lower concentrations, where the total count is relatively low. Based on the results, the model yields an average improvement of 5.43-fold in CV.

### Discussion

In this work, we have developed a label-free digital kinetic immunoassay that addresses the limitations of current single-molecule immunoassays, enabling specific, rapid, and ultrasensitive detection of molecular biomarkers in complex sample matrices including undiluted serum, plasma, and whole blood. By integrating a Gaussian Bayes algorithm, the assay fully exploits kinetic information to enhance both sensitivity and precision. We validated the LFSMiA using a commercially available and well-characterized IL-6 antibody pair, achieving sub-femtomolar detection in pure buffer, undiluted serum, and whole blood. The assay demonstrated an impressive dynamic range of eight logs, surpassing the performance of most existing technologies. The results of IL-6 detection across different sample matrices reveal that interfering substances in real biological samples can impact the limit of detection (LOD) and sensitivity of the assays. In addition, the LFSMiA for rapid NT-proBNP detection in plasma exhibited superior sensitivity, a broader dynamic range, and significantly shorter sample-to-answer time compared to the gold-standard ELISA. When applied to serum samples from heart failure patients, the LFSMiA showed excellent correlation with FDA-approved routine clinical diagnostic tests, demonstrating significant potential for clinical translation. The successful ultra-sensitive detection of multiple clinically important biomarkers in undiluted whole blood highlights the potential of the LFSMiA for point-of-care (POC) testing, as it eliminates the need for sample preparation and the associated risk of biomarker loss. The inexpensive, compact label-free microscope (LFSMiA-lite) that we developed and optimized for digital immunoassays proved to be as effective as the laboratory-based microscopy. Applied to whole blood samples from heart failure patients, LFSMiA-lite, using a 40 μL sample volume and a total assay time of 20 minutes, produced results that strongly correlated with those obtained using clinical diagnostic assays. Thus, the LFSMiA represents a highly promising platform for clinical diagnostics and offers significant potential for POC diagnostics.

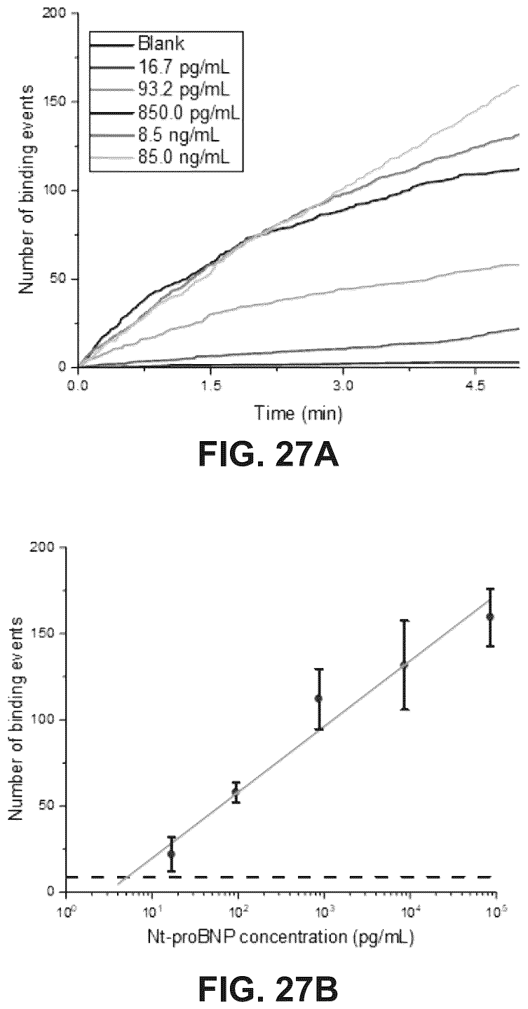

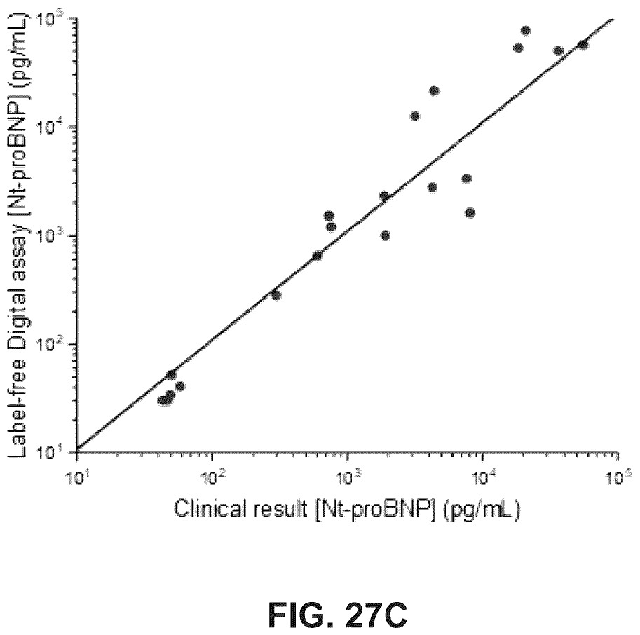

The focus of this work is to introduce a new single-molecule technology with acceptable analytical performance that can overcome the limitations of signal amplification with labeling. LFSMiA reports the total binding events N (T) of detection antibody on the surface as output signal, which is defined as

$$N ( T ) = {\int }_{0}^{T} {{{k}_{on} [ A ]}_{t} [ P ]}_{t} dt$$

where kₒₙ is the association rate constant of the detection antibody, [A]ₜ and [P]ₜ are the concentrations of free analyte and detection antibody at time t. Therefore, the signal of LFSMiA would still increase even after detection antibody binding reaches dynamic equilibrium, which makes it superior to conventional endpoint assay for detecting low-abundance biomarkers. It is worth noting that the accuracy and sensitivity of LFSMiA are primarily limited by the number of the binding event numerated, which could be improved by increasing FOV or reaction time. We compared the results for the reaction time of 5 min or 10 min and it shows that longer reaction time would improve the accuracy and sensitivity (FIG. 27). However, clinically acceptable assay time and the effects of antibody dissociation restricts the maximum length of reaction time. Increasing FOV requires a more expensive camera with more pixels and high data recording rate. A more powerful light source is also needed to enable single-molecule sensitivity in larger FOV. Fortunately, imaging technologies powered by machine learning could possibly enable the detection of IgG antibody at lower exposure time, allowing larger FOV imaging with higher frame rates.

LFSMiA still suffer from some limitations. In the current set-up, LFSMiA uses an incident illumination intensity of ˜2 kW/cm² to image single detection antibody with acceptable signal-to-noise ratio (SNR). Higher SNR can be obtained by increasing the incident intensity, which could improve the accuracy of dynamic counting of single binding events. However, the protein molecules on the surface could be damaged due to photothermal effect.

As a sandwich assay, the affinity of antibodies would influence the performance of LFSMiA. Most commercial antibodies with nanomolar K_{D} are compatible with LFSMiA. However, antibody with kₒₙ lower than 10⁴ M⁻¹s⁻¹ would result in insufficient number of binding events detected in the assay time. We could improve the binding kinetics of such antibody by construct multivalent antibody via DNA assisted super assembly technique.

Despite these limitations, LFSMiA shows obvious advantages compared with existing label-based digital immunoassays, such as SiMoAs, SIMREPS and SMAC. The label-free nature of LFSMiA avoids the cost associated with fluorescence probes. By adding an air objective on the top of imaging area, LFSMiA could be simply achieved on commercial SPR systems, which could ease the adoption of LFSMiA. The ability to detect individual biomarker in undiluted complex clinical fluids simplify the workflow of clinical testing and is POC compatible. Compared with label-free methods like (iSCAMS) and SPR, LFSMiA excels at distinguishing between specific binding of detection antibody to analyte and nonspecifically absorbed biomolecules from the complex media. Therefore, LFSMiA is a reliable technique for fundamental biological research with the potential of moving digital immunoassay into clinical application.

### Methods

### Reagents and Materials

Human cardiac troponin T (cTnT, catalog no. 8RTT5) was purchased from HyTest Ltd. Bovine serum albumin (BSA, catalog no. A7638-5G) and immunoglobulin G (IgG, catalog no. 12511-10 MG) were purchased from Sigma-Aldrich. Human colostrum immunoglobulin A (IgA, catalog no. SIA1901-R22) and human plasma immunoglobulin M (IgM, catalog no. IP2020-03) were purchased from Athens Research and Technology. IL-6 monoclonal capture antibody (clone no. MQ2-13A5, catalog no. 14-7069) and detection antibody (clone no. MQ2-39C3, catalog no. 13-7068) were purchased from ThermoFisher Scientific. Recombinant human IL-6 protein (catalog no. 206-IL) was purchased from R&D Systems. PSA monoclonal capture antibody (clone no. M612165, catalog no. 10-P21A), detection antibody (clone no. M612166, catalog no. 10-P20A) and purified native human PSA protein (catalog no. 30-1205) were purchased from Fitzgerald Industries. Human NT-proBNP capture antibody (catalog no. BRJNBNPS108), detection antibody (catalog no. BRJNBNPS102) and recombinant protein (catalog no. GRCBNPS101) were purchased from Fapon International Limited. Reagent diluent (catalog no. DY995), streptavidin conjugated horseradish peroxidase (streptavidin-HRP, catalog no. DY998), stop solution (catalog no. DY994) and high-binding microplate (catalog no. DY990) were purchased from R&D systems. Gender unspecified pooled K2EDTA plasma from healthy human donors and gender unspecified pooled bovine whole blood were purchased from BioIVT Elevating Science. Heat-inactivated horse serum (catalog no. 26050) was purchased from ThermoFisher Scientific. ACS grade denatured ethanol (catalog no. BDH1158) was purchased from VWR International. N-hydroxysuccinimide (NHS, catalog no. 130672), N-(3-dimethylaminopropyl)-N-ethylcarbodiimide hydrochloride (EDC, catalog no. 03450), Amicon Ultra-0.5 mL centrifugal filters, 6-mercapto-1-hexanol (catalog no. 725226) and 8-mercaptooctanoic acid (catalog no. 675075) were purchased from Sigma-Aldrich (St. Louis, MO). Sylgard 184 Clear Silicone Elastomer Kit (catalog no. DC4019862) was purchased from Krayden Inc.

### Microfluidic Sensor Chip Fabrication

47 nm gold coated 24×50 mm² glass coverslip was used as the substrate of the sensor chip. The gold substrate was thoroughly rinsed with ethanol and deionized water sequentially and annealed using hydrogen flaming. The annealed gold substrate was then immersed overnight in an ethanol solution containing 100 μM 8-mercaptooctanoic acid (MOA) and 10 mM 6-mercapto-1-hexanol (MCH). After the gold substrate was thoroughly rinsed with ethanol and deionized water, a layer of 50 μm thick double-sided tape with a 3×36 mm² straight channel was adhered to the gold surface. Then, a 24×40 mm² glass coverslip with 2 drilled holes located at the 2 ends of the straight channel was placed onto the double-sided tape and pressed to form an enclosed microfluidic channel. To enable convenient fluid dispensing, 2 Polydimethylsiloxane (PDMS) pieces each with a punched through hole were bonded to the top surface of the glass coverslip with the through holes aligned. Finally, epoxy glue was applied to all the edges both of the top glass coverslip and the 2 PDMS pieces and cured for 1 hour at room temperature to secure and seal the sensor chip.

### Sensor Surface Functionalization

The capture antibodies for IL-6, NT-proBNP and PSA were preprocessed by buffer exchanging into MOPS buffer (5 mM MOPS, pH 7.4) with 5 spin cycles using Amicon centrifugal filters. 100 μL of an aqueous solution containing 5 mM EDC and 10 mM NHS was injected into the microfluidic channel and incubated for 5 min. Later, this procedure was repeated 2 more times to activate the carboxylic acid functional groups on the sensor surface. The channel was flushed with 100 μL MOPS buffer. In a 1-hour total incubation time, 100 μg/mL capture antibody in MOPS buffer was injected 3 times respectively at 0, 5 and 15 min with 20 μL volume used each time. Then, 100 μL aqueous solution containing 1 M ethanolamine with pH of 9.6 was injected to quench the unreacted NHS esters. Lastly, 100 μL of 1× phosphate buffered saline (PBS) was injected to flush the channel. The sensor chips were prepared prior to experiments and used within the same day.

### Human IL-6 and PSA Detection

For IL-6 detection in pure buffer, human recombinant IL-6 was spiked into 1× reagent diluent (1% BSA in 1×PBS) to reach final concentrations of 1, 10, 100, 10³, 10⁴, 10⁵, 10⁶, and 10⁷ fM. For IL-6 detection in horse serum, 1, 10, 100, 10³, and 10⁴ fM of recombinant IL-6 were spiked into the horse serum. For IL-6 or PSA detection in bovine whole blood, the recombinant proteins of IL-6 or PSA were spiked so that the final plasma concentrations for each whole blood samples were 1, 10, 100, 10³, and 10⁴ fM. The corresponding samples matrices without spiking were used as blank control to measure their background level. These spiked samples were injected through the sensor chips for 2 hours with a flow rate of 10 μL/min. Following a flushing step with 1× PBS, the chips were installed onto PSM. 3 mL of 10 nM detection antibody in 1×PBS was flowed through the microfluidic channel for 10 min driven by gravity pump while PSM was recording images of the sensor surface in real time. The PSM images were then analyzed using an in-house dynamic tracking algorithm to count the number of detection antibodies binding to the sensor surface.

### Human NT-proBNP Detection and Clinical Evaluation

To generate the NT-proBNP calibration curve in human plasma and bovine whole blood, recombinant human NT-proBNP protein was spiked into the pooled human plasma or bovine whole blood to obtain spike-in concentrations of 1, 10, 100, 10³, 10⁴ and 10⁵ pM. Baseline level of NT-proBNP in the human plasma pool used for spiking were 0.97 pM, which was measured using conventional ELISA. As such, the corrected concentrations were 1.97, 10.97, 100.97, 10³, 10⁴ and 10⁵ pM. These standard samples were injected through the sensor chips for 10 min with a flow rate of 50 μL/min and 5 μL/min for plasma and whole blood sample respectively, and then measured using PSM following the same procedure as in IL-6 detection. Under a protocol approved by the Mayo Clinic Institutional Review Board and Biospecimens Subcommittee (IRB #19-002558/Bio00017399), 28 patient serum and whole blood samples were deidentified and provided by Mayo Clinic Arizona and measured using the LFSM-immunoassay. The patient samples were residual volume from routine clinical testing of NT-proBNP by Roche's Elecsys proBNP II assay. They were transported in icebox to our lab, stored at 4° C. and tested within 6 days.

### Conventional ELISA Measurement of NT-proBNP Baseline

To measure the baseline NT-proBNP level in the spiking human plasma pool, a 96 well microplate was coated overnight with 100 μL of 2 μg/mL capture antibody of NT-proBNP in 1×PBS. After washed 3 times with 200 μL of 0.05% PBST (1×PBS containing 0.05% v/v Tween 20), the plate wells were blocked with 200 μL 1× reagent diluent for 1 hour. Then, a series of horse serum standard samples with spike-in NT-proBNP concentrations of 25, 50, 100, and 200 pg/mL were prepared. Horse serum without spiking was used as 0 pg/mL blank control. A mixture of 50 μL NT-proBNP-spiked horse serum standard sample or the human plasma and 50 μL biotinylated detection antibody (2 μg/mL) was incubated in the wells for 1 hour. Following another washing step as previously, 100 μL streptavidin-HRP solution was incubated in the wells for 20 min. Washed again with 0.05% PBST, the wells were reacted with 100 μL TMB substrate solution for 20 min. Lastly, 50 μL stop solution was added, followed by reading the absorbance of each well using a microplate reader (EnVision 2104, Perkin Elmer).

### Experimental Setup

For lab used PSM, a 120 mW diode laser (L660P120, Thorlabs) with a central wavelength at 660 nm was used as the light source. The light was first collimated by a 20× objective and the beam size was reduced by a lens group. The sized beam was then focused on the prism surface by a short-focus lens with an incident angle of 71° to reach SPR. The scattered light from the biomolecule and gold surface was collected by a 60× air objective (Olympus, LUCPLFLN60X, NA=0.7) equipped with a 180 mm tube lens to form an image on a CMOS camera (MQ013MG-ON, XIMEA). More details could be found in FIG. 10.

For LFSMiA-lite, the basic principle was similar to the lab used setup. However, we changed the light source to a cheaper 100 mW diode laser and 60× air objective. We also simplified the lens group and the structure of the setup to minimize the size and cost for the lite setup.

### Image Processing

The raw image sequence was recorded by XIMEA Cam Tool and then was processed by custom written Matlab scripts (further details are provided herein).

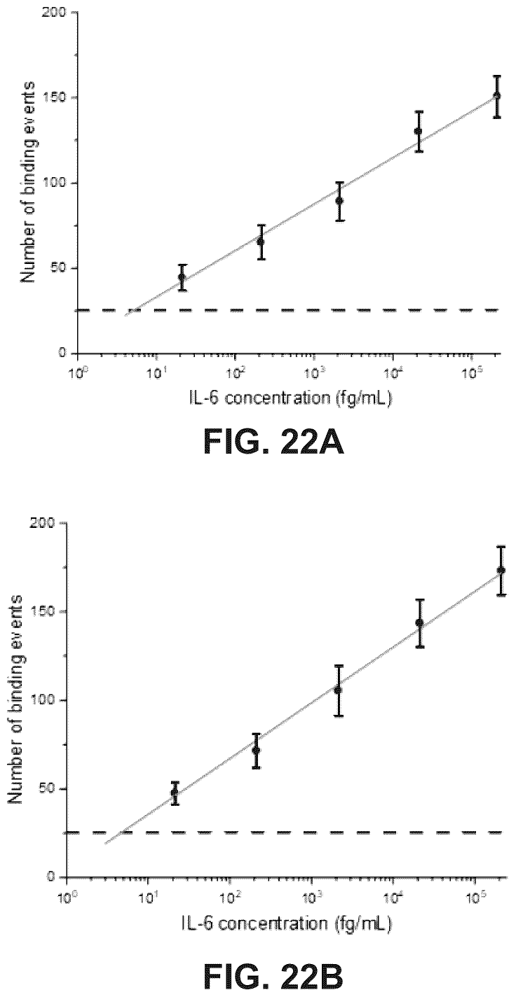

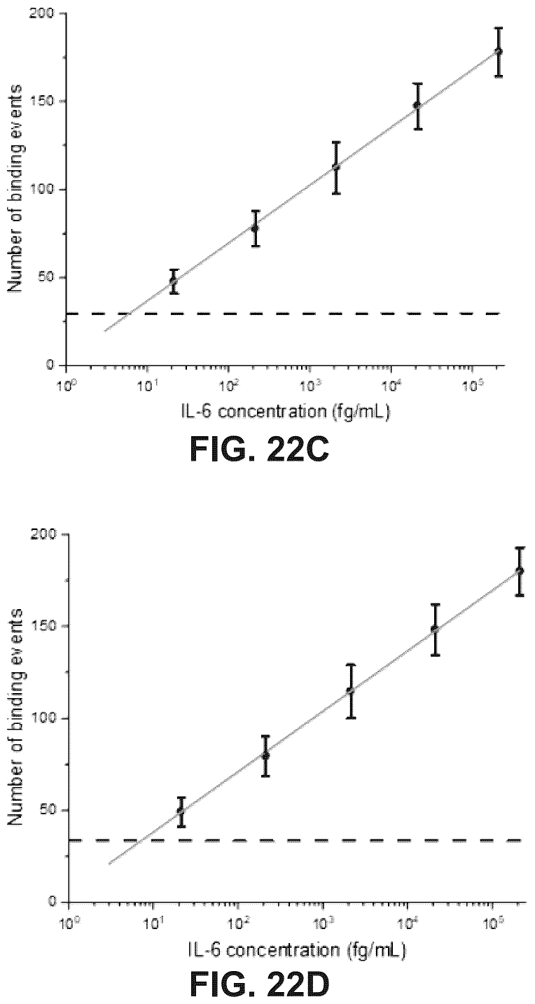

TABLE 1
The limit of detection (LOD), r-square (COD) and coefficient
of variance (CV) of the standard curves of IL-6 detection
in pure buffer for different binding frequency thresholds
(times in 10 mins) shown in FIG. 22.

| Threshold | LOD | COD | CV |
|---|---|---|---|
| 1 | 5.38 pg/mL | 0.98224 | 12.46% |
| 2 | 4.88 pg/mL | 0.99251 | 11.67% |
| 5 | 6.18 pg/mL | 0.99926 | 11.37% |
| 10 | 7.31 pg/mL | 0.99941 | 11.76% |

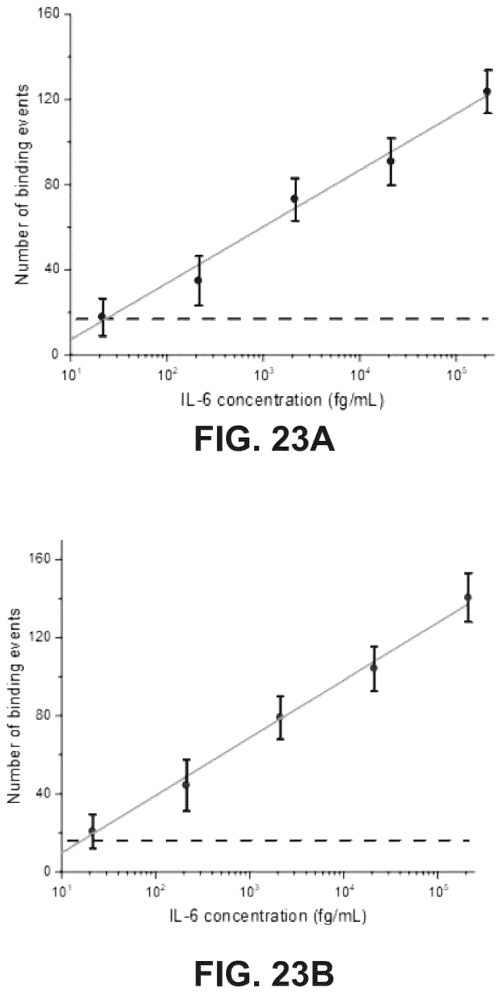

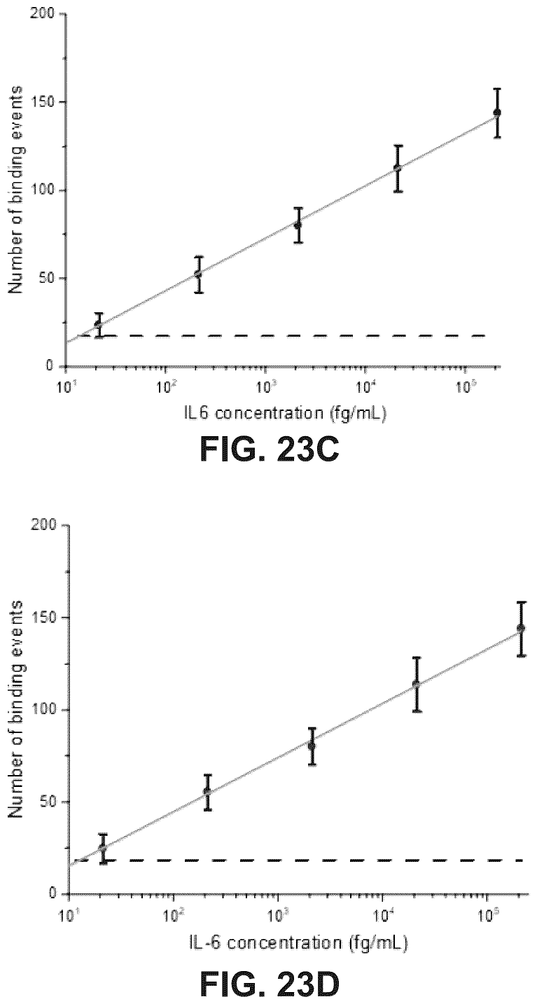

TABLE 2
The limit of detection (LOD), r-square (COD) and coefficient
of variance (CV) of the standard curves of IL-6 detection
in bovine whole blood for different binding frequency
thresholds (times in 10 mins) shown in FIG. 23.

| Threshold | LOD | COD | CV |
|---|---|---|---|
| 1 | 23.22 pg/mL | 0.98503 | 23.08% |
| 2 | 16.70 pg/mL | 0.99445 | 20.86% |
| 5 | 13.60 pg/mL | 0.99873 | 16.57% |
| 10 | 13.18 pg/mL | 0.99703 | 16.83% |

Protocol of LFSM-immunoassay. The gold substrate was first dipped in an ethanol solution with 100 μM 8-mercaptooctanoic acid (MOA) and 10 mM 6-mercapto-1-hexanol (MCH) overnight. After rinsed by ethanol and DI water for three times, the gold chip was made into microfluidic sensor chip following the procedure described in the main text. 60 μL EDC and NHS mixed solution (5 mM and 10 mM in DI water) was injected 3 times into the microfluidic channel at 0, 5 and 10 min in a 15-min total incubation time. The sensor was then cleaned by 200 μL MOPS buffer. Capture antibody with a concentration of 100 μg/mL in MOPS buffer was injected three times to improve the efficiency of surface modification. The capture antibody coated sensor was then exposed to test sample, after quenched by 1 M ethanolamine with PH of 9.6. After washing by 500 μL PBS buffer to remove the bubble in the channel, 3 mL detection antibody solution was flowed through the sensor for 10 min, during which the binding was recorded by PSM imaging.

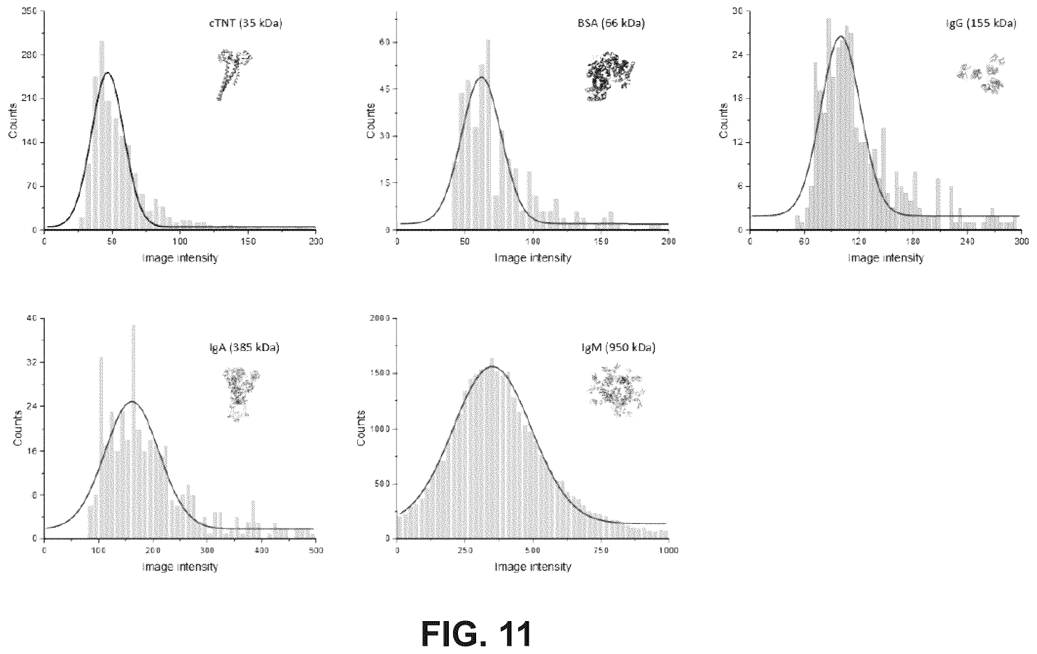

Binding event detection. The binding event was extracted following the procedure shown in FIG. 20. The noise in the image was first reduced by applying 25 frames rolling averaging of the raw video starting at frame m, Nₘ^{m+25}, and then normalizing each frame of the averaged video in terms of their mean pixel value to avoid the intensity change from the light source. A differential image sequence was obtained by subtracting each normalized frame from its subsequent frame (Nₘ₊₂₅^{m+50}−Nₘ^{m+25}), which removed the rough background and revealed the binding and unbinding events. The differential images were then convoluted with Haar-like arrays to be transferred into a probability image (PI), which extracted the morphological characteristics in the image². Pixels with intensity higher than the mean intensity+3×standard deviation (s.d.) of the whole PI were selected as candidate pixels. For each pixel of the differential image with the same coordinates as the candidate pixels of the PI, a fixed neighborhood (11×11 pixel²) was extracted and fitted with a 2D Gaussian function to get the precise center location and intensity of the particle. The 2D Gaussian model is defined as:

$$f ( x , y ) = {Ae}^{[ \frac{{( x - {x}_{0} )}^{2}}{2 {σ}_{x}^{2}} - \frac{{( y - {y}_{0} )}^{2}}{2 {σ}_{y}^{2}} ]} + c$$

where (x₀, y₀) is the position of the binding event in the differential image, (σₓ, σ_{y}) are standard deviations of the 2D Gaussian model which contains the morphological information of the binding particle, A is the intensity of the binding particle and c is the offset constant. The pixels would be rejected if the ratio between σₓ and σ_{y} was smaller than 0.7 or larger than 1.43 as the binding particle is not single molecule¹. The binding event of single particle appears as a brightening Gaussian blob and then gradually disappears in several consecutive frames (FIG. 21). To avoid over-counting particles, binding events from consecutive frames whose center positions fall within a fixed distance (|x₁−x₂|+|y₁−y₂|≤3 pixels) would be grouped as one binding event. To extract the most accurate image intensity of the binding event, a function I(t), whose value would grow linearly up to a maximum and then decrease linearly, was fitted to the profile of the binding intensity VS time and the maximum value of the fitted model was chosen as the intensity of the binding event. I(t) is defined as:

$$I ( t ) = M - \vert k ( t - {t}_{0} ) \vert$$

where I is imaging intensity, M is the accurate intensity of binding event, k is a constant represent the speed of the binding event and t₀ is the accurate time the particle hit the surface. The binding event which did not appear in 5 continuous frames would be considered as nonspecific binding and deleted. Based on the relationship between MW and image intensity (FIG. 11), the detected binding events would be considered as detection antibody binding to the analyte if their image intensity were within the range of 60 to 240 (consider the formation of dimers).

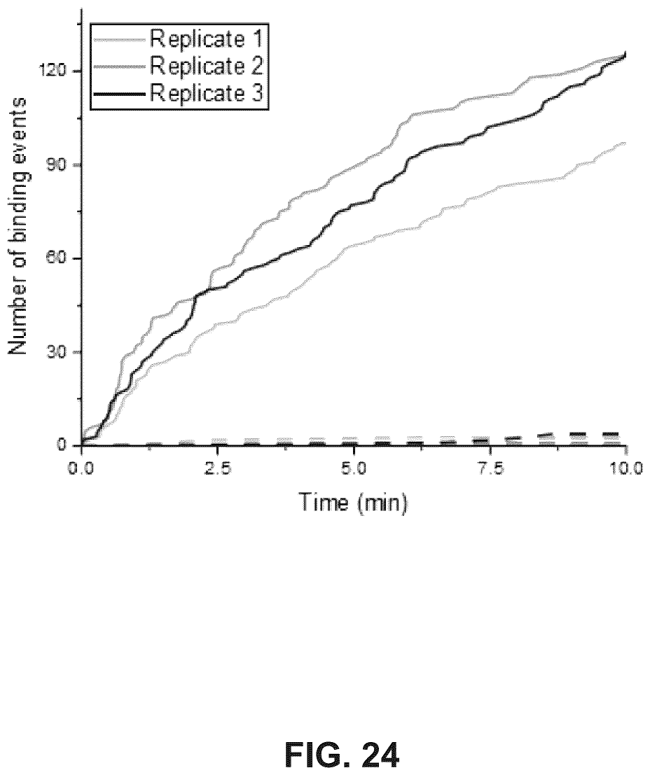

Determination of the threshold for binding time. As the clinical sample contains proteins with various sizes, label-free imaging could not confidently distinguish the dissociation event of detection antibody and that of protein with similar size after the sensor was incubated in complex sample. We analyzed the binding of IL-6 detection antibody to surface captured IL-6 (FIG. 24). After immobilizing capture antibody, BSA solution was used to passivate the surface. 100 fM IL-6 in pure buffer was flowed over the sensor surface for 1 hour. Then 3 mL IL-6 detection antibody with concentration of 10 nM was introduced into the system for 10 mins, which was recorded by PSM imaging. Similar to the algorithm of detecting binding event, the only difference in detecting dissociation event was that only the dissociation events with an image intensity in the range of −60 to −240 were considered as detection antibody leaving the surface. As shown in the FIG. 24, the number of dissociation events can be ignored compared with the number of binding events. Therefore, we only counted the binding event in our assay time.

As shown in support video, the main difference between nonspecific binding and specific binding is that the nonspecific binding would hit the same place on the sensor surface more times. To estimate the probability of two detection antibody binding to the same area on the surface, we can assume that the surface is composed of 1000 blocks with evenly distributed antigen molecules. So, the probability for a detection antibody binding to a certain block is 0.001. The probability of the event where n detection antibodies in a total of 100 detection antibodies bind to the same block can be calculated as

$$p ( n , 100 ) = {C}_{100}^{n} {0.001}^{n} 0.99 {9}^{100 - n}$$

The probabilities for n=0, 1 and 2 are 0.9048, 0.0906 and 0.0045 respectively. Therefore, the probability for several detection antibodies binding to the same position is low and a threshold of binding frequency can be used to distinguish specific binding and nonspecific binding. We used different thresholds of binding time to process the data of IL-6 detection in pure buffer and whole blood. The standard curves were shown in FIGS. 22 and 23. The details of the standard curves of pure buffer and whole blood were shown in Tables 1 and 2, respectively. Comparing the LOD, COD and CV of different thresholds, the threshold value of “5 times in 10 minutes” has at least two-thirds of the parameters better than the other threshold values in both pure buffer and whole blood group.

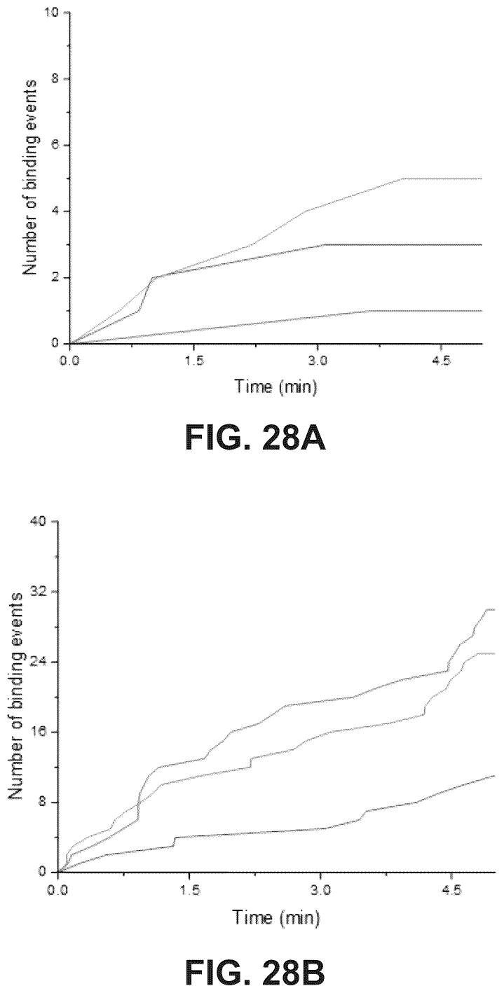

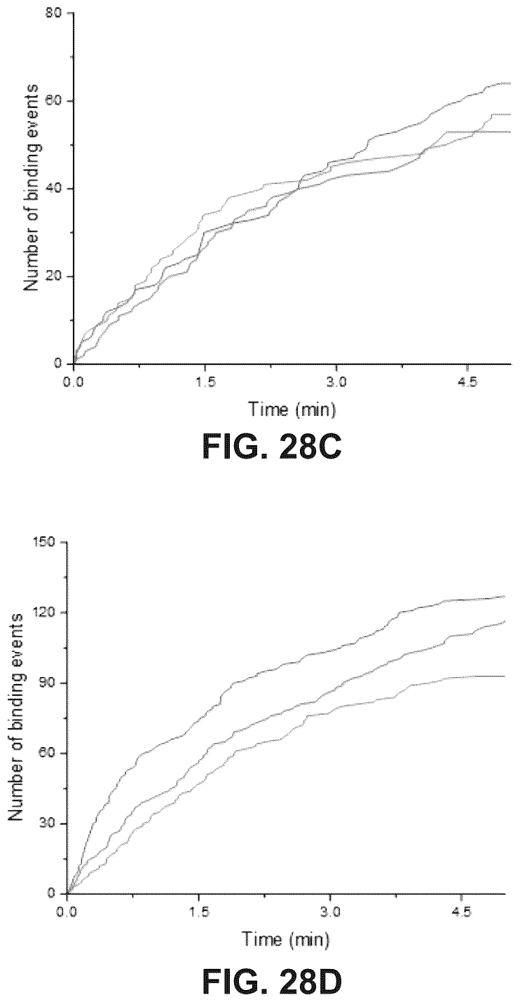

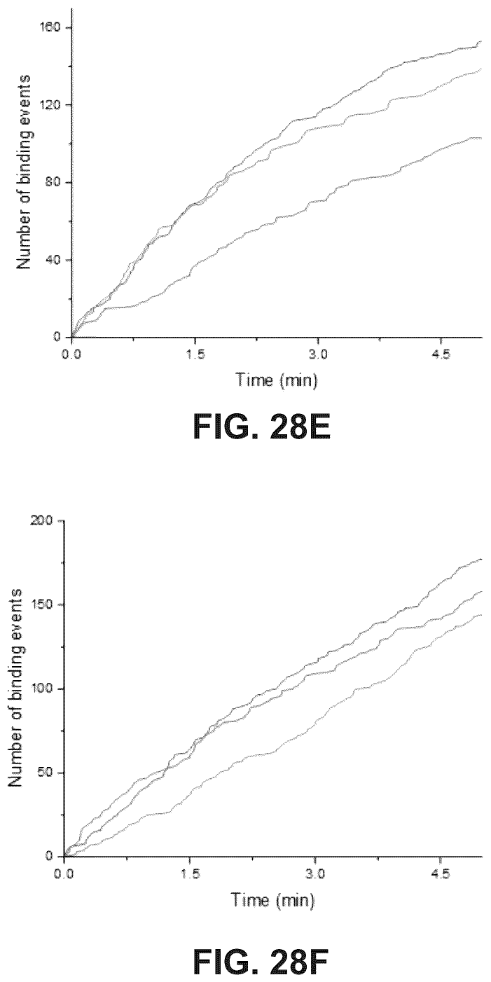

Comparison of LFSM-immunoassay at different counting time. We studied the influence of counting time on the detection limit and the precision by comparing the standard curves of NT-proBNP detection under clinical setting for counting time of 5 and 10 mins. The first 5 mins data in FIG. 6 was processed in the same way and shown in FIG. 27 (details of each replicate could be found in FIG. 28). The detection limits are 5.22 pg/mL for 5 mins and 4.70 pg/mL for 10 mins. Comparing with the fitting precision of 10 mins counting time, the r-square of 5 mins drops from 0.99 to 0.97 (FIG. 27b). The correlation coefficient between our method and Roche's assay also drops from to 0.99 to 0.95 (FIG. 27c). Therefore, longer counting time within clinically acceptable range would improve the sensitivity and precision of LFSM-immunoassay by enabling more binding events to be counted⁵.

The effect of detection antibody concentration on NT-proBNP detection. We compared NT-proBNP detection results for detection antibody concentrations of 10 nM and 50 nM. The number of binding events in blank experiment are similar for the two concentrations (FIG. 25a). However, the response signal for the 50 nM is bigger than the 10 nM, after the sensor was incubated in human plasma with 100 fM NT-proBNP for 10 mins.

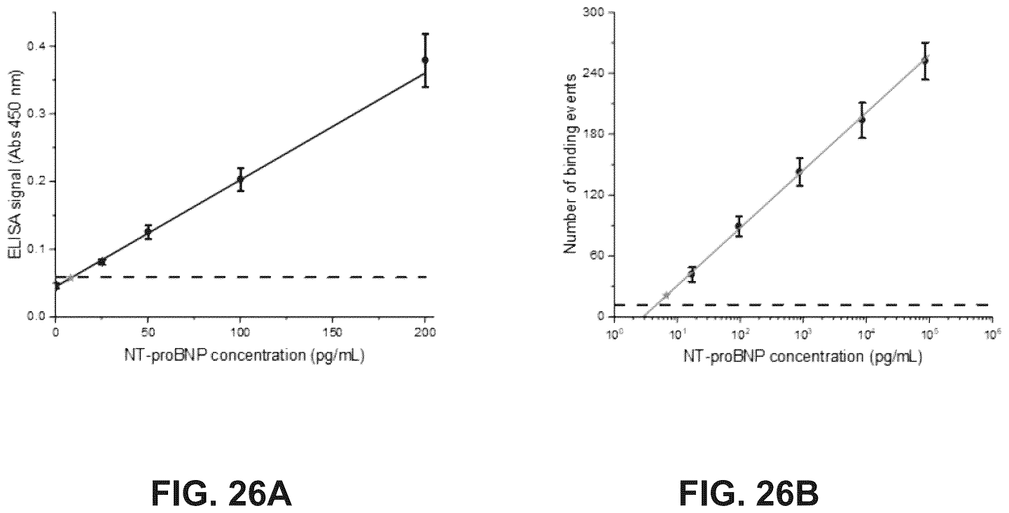

NT-proBNP concentration in pooled human plasma. As NT-proBNP is a stable protein and exists in the blood of both patients and healthy people, the endogenous concentration of NT-proBNP in pooled human plasma should be determined to get the accurate spiked concentration. The concentration of NT-proBNP in human plasma pool was measured to be 8.23 pg/mL by conventional ELISA (FIG. 26a). We also used our method to measure the baseline level of NT-proBNP in pooled human plasma, which was determined to be 6.71 pg/mL (FIG. 26b). Based on these results, the recovery was found to be 18.5%.

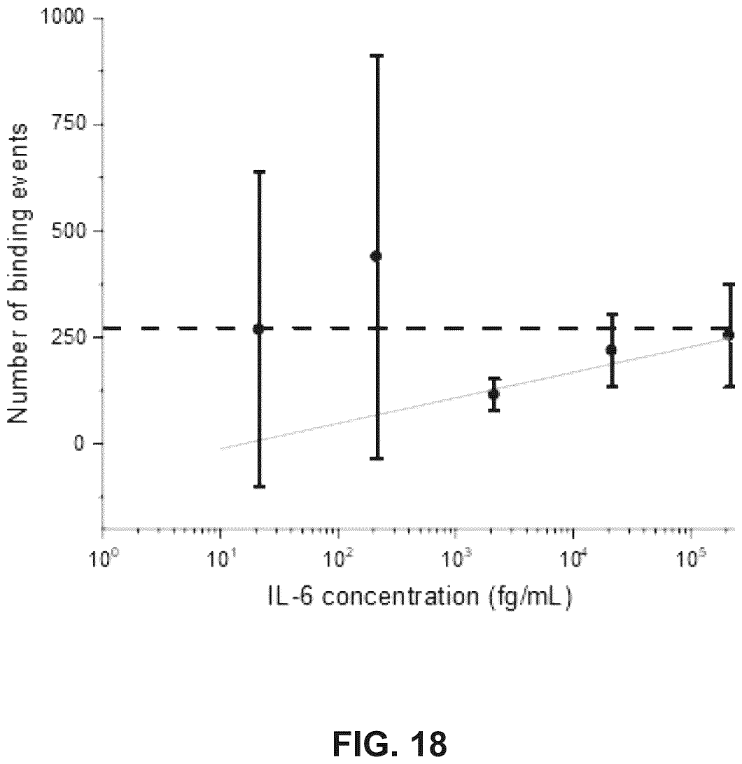

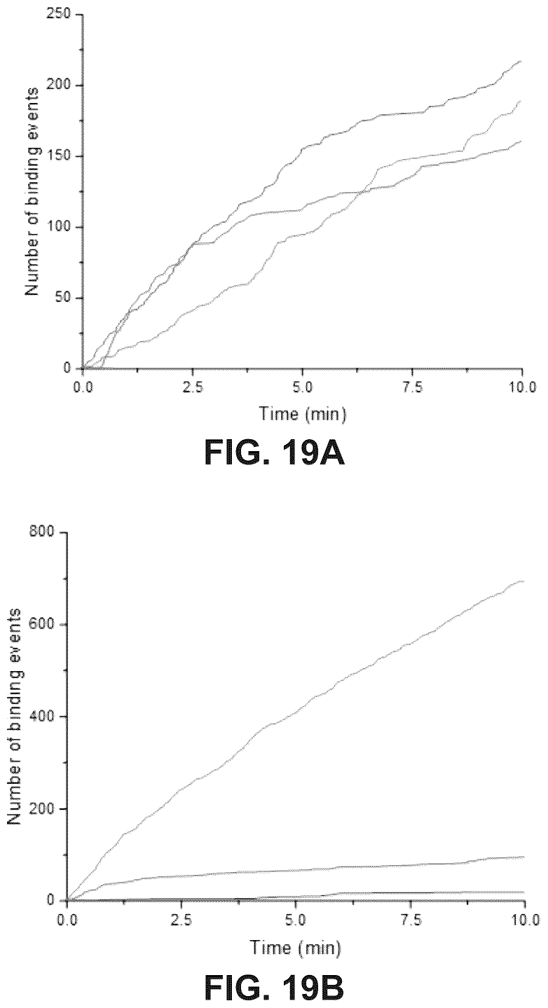

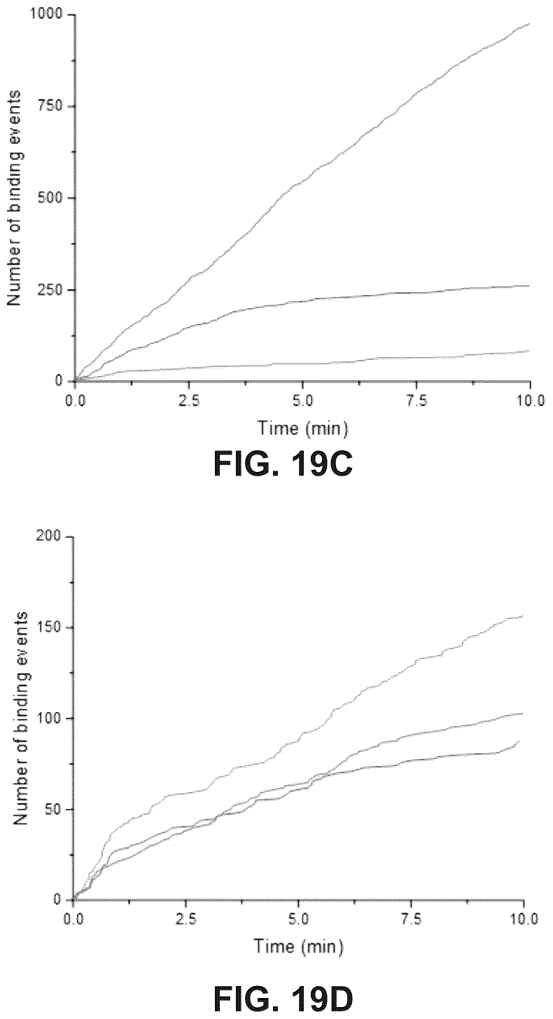

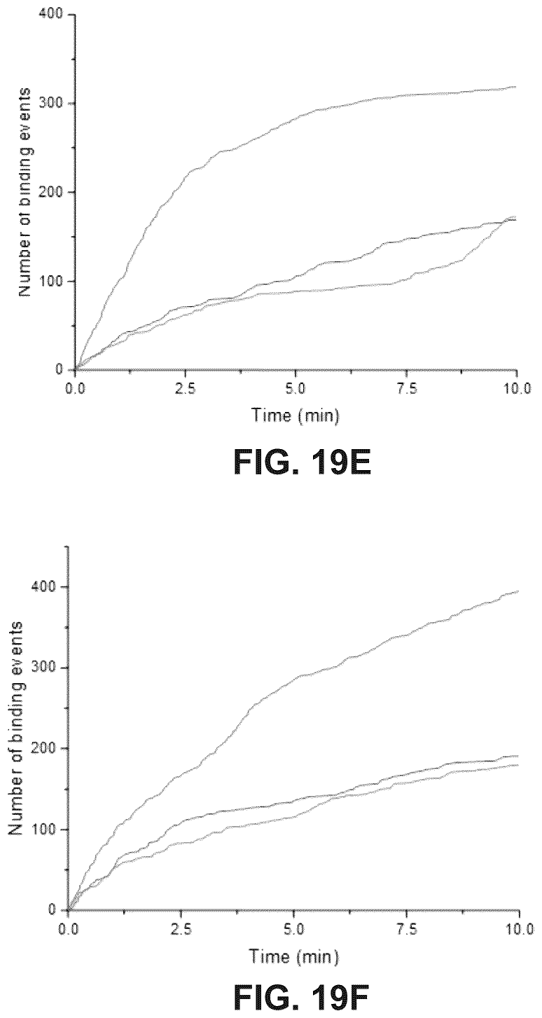

Comparison of standard curves of IL-6 detection in whole blood with/without filtering by our real-time counting algorithm. To demonstrate the ability of our algorithm to remove nonspecific binding, standard curve of IL-6 detection in whole blood without applying intensity and binding time filtering to the binding events was shown in FIG. 18 (Details of each data points could be found in FIG. 19). Based on the results, the IL-6 detection in whole blood could not be realized without the filtering of our real-time counting algorithm.

Gaussian process model for binding event. From a statistical perspective, we model the real-time binding event with a stochastic process indexed by time, and the measured binding counts are viewed as observations of the underlying binding process at the designated time points. As the binding process is continuous over time and it contains the dependence between count values at any two time points, a Gaussian process becomes a natural fit for the binding process. Gaussian process modeling is a famous supervised machine learning method due to its flexibility to generate smooth realizations. The associated covariance kernel function controls the dependence among function values, which allows the estimation to borrow information from the neighborhood. In particular, we implemented Gaussian process model under a Bayesian framework to not only obtain the binding count estimates but also provide quantification of uncertainties (posterior standard deviation) in the obtained estimates.

Gaussian process modeling of binding event. We describe the binding process as a continuous real-valued function of time, defined as ƒ: [symbol] → [symbol] , a mapping from the time interval [symbol] of interest to the real line [symbol] . We assume that the binding count function follows a zero-mean Gaussian process ƒ: [symbol] → [symbol] as ƒ˜GP(0, c(⋅, ⋅)) with a bivariate covariance kernel function c(⋅, ⋅): [symbol] × [symbol] → [symbol] ⁺, here [symbol] ⁺ denotes the positive part of the real line. The covariance kernel function only produces positive correlation by default. The type of covariance kernel function determines the correlation level between the binding counts measured at two different time points, which will be chosen later. As there is no evidence that the binding process follows a certain trend, we set the mean of Gaussian process to be constant zero over time. By the definition of a Gaussian process, at the designated time points {t₁, . . . , tₙ}, the evaluated binding counts [ƒ(t₁), . . . , ƒ(tₙ)] jointly have a n-dimensional multivariate normal distribution as

$${f}_{n} := [ \begin{matrix}f ( {t}_{1} ) \\ f ( {t}_{2} ) \\ ⋮ \\ f ( {t}_{n} )\end{matrix} ] ∼ N ( {0}_{n} , {𝒯}_{n} ) , \qquad 1$$

where the mean vector is a n-dimensional zero vector 0ₙ and the variance-covariance matrix [symbol] is a n×n symmetric and positive definite matrix with its (i, j)-th element [symbol] =c(tᵢ, tⱼ) for 1≤i,j≤n. As the binding process is expected to be relatively smooth, we consider a squared-exponential covariance kernel function of the form

$${c}_{SE} ( x , y ; ℓ ) = exp \{ - \frac{{( x - y )}^{2}}{2 {ℓ}^{2}} \} , for any x , y ∈ 𝒯 , \qquad 2$$

where [symbol] >0 is the associated length-scale parameter that controls the correlation level between the function evaluations at different locations. The squared-exponential kernel function is commonly used to generate smooth GP realizations which contains strong dependence. Implementing the squared-exponential kernel in Equation 1 yields that ƒₙ˜N(0ₙ, [symbol] ), where [symbol] is the correlation matrix with its (i, j)-th element [symbol] =c_{SE}(x, y; [symbol] ) for any 1≤i, j≤n.

Bayesian inference. The main goal is to recover the binding counts from the noised observations as well as to quantify the uncertainty in the estimates. We specifically take a Bayesian approach for estimation due to its advantage in uncertainty control. Given the data Y, one may define a likelihood function P_{θ}(Y) that links the data and the assumed model associated with unknown parameter θ to be estimated. Under a Bayesian framework, one may treat the unknown parameter θ as random quantities that are endowed with some distribution Π(θ), named as the prior distribution. The prior distribution is determined to incorporate the a priori knowledge on the parameter. Bayesian inference is conducted through the posterior distribution which combines the prior information and the likelihood. That being said, the posterior distribution is obtained by applying the Bayes' rule,

$$Π ( θ \vert Y ) = \frac{{P}_{θ} ( Y ) Π ( θ )}{\int {P}_{θ} ( Y ) Π ( θ ) d θ} \qquad 3$$

The posterior distribution Π(θ|Y) is a well-defined distribution of the parameter θ conditioning on the data Y. As follows, we will specify the likelihood function that links the binding count observations and the Gaussian process model. The prior distributions of the associated unknown parameters shall be provided as follows.

Gaussian process hierarchical model. We denote the collected data points {yᵢ, i=1, . . . , n} of size n are noisy observations of the binding process at given time points {tᵢ, i=1, . . . , n}. We also assume that the measurement error, systematic error, and other unknown errors are random and additive to the true binding count values. Under the above assumptions, we now propose our Bayesian hierarchical model

$${y}_{i} = f ( {t}_{i} ) + {ε}_{i} , {ε}_{i} ∼ N ( 0 , {σ}^{2} ) , \qquad 4$$

$$[ f ( {t}_{1} ) , … , f ( {t}_{n} ) ] ∼ N ( {0}_{n} , {2}^{} {𝒯}_{ℓ} ) , \qquad 5$$

$${σ}^{2} ∼ IG ( {a}_{σ} , {a}_{σ} ) , {2}^{} ∼ IG ( , ) , ℓ ∼ Unif ( {a}_{ℓ} , {b}_{ℓ} ) . \qquad 6$$

Equation 4 describes the likelihood function that links the observations {yᵢ, i=1, . . . , n} to the assumed Gaussian process values {ƒ(tᵢ), i=1, . . . , n} evaluated at time {tᵢ, i=1, . . . , n}. We assume that the additive unknown random errors E's are independently and identically distributed (i.i.d.) with a centered normal distribution with some positive variance parameter σ². The variance σ² is assumed to be unknown and is assigned with an Inverse-Gamma (IG) prior. As the binding function ƒ is modeled with a Gaussian process associated with a squared-exponential covariance kernel function, Equation 1 implies a multivariate normal prior distribution for [ƒ(t₁), . . . , ƒ(tₙ)] defined in Equation 5, where the correlation matrix [symbol] is defined in Equation 2, and the associated length-scale parameter [symbol] is also assumed to be unknown. We consider a Uniform prior on [symbol] . In addition, in Equation 5, the prior covariance matrix contains a positive scale parameter [symbol] to control the magnitude of the variances, as the matrix [symbol] only controls correlations. The scale [symbol] is unknown and is endowed with an Inverse-Gamma prior. More details on prior distributions are discussed in the hyper parameter specification section.

Hyperparameter specification. To simplify the fitting process, we standardize the observed binding counts to ensure that the observations are on the same scale in different cases, and also scale the time points to be all within [0, 1]. Specifically, for the error variance parameter σ², we specify that σ²˜IG(α_{σ}, α_{σ}) with α_{σ}=0.01. This prior is considered as weakly informative⁶ in the sense that the prior does not enforce a strong subjective preference on the value of σ², thus the posterior lets the data to update σ². The standardized binding counts in different cases range mainly in [−2, 2] with a unit standard deviation. A proper prior on ƒₙ shall assign the major amount of the prior probability on the values of ƒₙ which roughly are the same scale as standardized binding counts. It is easy to see that the values of ƒₙ depend on the scale of t, the prior standard deviation. Therefore, the prior distribution of [symbol] is determined such that ƒₙ is also on the same scale as the standardized binding counts. Based on this discussion, we consider [symbol] ˜IG( [symbol] , [symbol] ) with [symbol] =5 so that the prior mean is 1.25 and the prior standard deviation is approximately 0.7, also, there is around 0.9 prior probability that the prior standard deviation of ƒₙ is less than 1.5. Our empirical results indicate that the performance of the model is not sensitive to the choice of [symbol] provided [symbol] ∈(2, 10). At least, for the length-scale parameter [symbol] associated with the Gaussian process, we consider the prior choice [symbol] ˜Unif( [symbol] , [symbol] ) with [symbol] =0.1, [symbol] =2. This setting allows for the correlation between two furthest time points in [0, 1] varying in (5*10⁻⁵, 0.6), allowing both a weak and strong long-range dependence possibly in the covariance matrix. The Uniform prior distribution is also noninformative so that the posterior distribution lets the data update the parameter [symbol] .

Posterior Inference. For simplicity of notation, we rewrite the likelihood function in Equation 4 in a matrix form. Denoted all of observed binding counts by Y=[y₁, . . . , yₙ] at the time points T=[t₁, . . . , tₙ], then the likelihood function P_{ƒ}_{ₙ}(Y)=N(Y; ƒₙ, σ²Iₙ), we denote by Iₙ the identity matrix of dimension n. Here we use N(Y; ƒₙ, σ²Iₙ) to denote the normal distribution of Y with the mean vector (ƒₙ) and the variance-covariance matrix (σ²Iₙ). Applying Bayes' rule to the hierarchical model in Equation 4-6 yields the joint posterior distribution

$$Π [ {f}_{n} , {σ}^{2} , {2}^{} , ℓ \vert Y , T ] ∝ N ( Y ; {f}_{n} , {σ}^{2} {I}_{n} ) N ( {f}_{n} , {0}_{n} , {2}^{} {𝒯}_{ℓ} ) IG ( {σ}^{2} ; {a}_{σ} , {a}_{σ} ) IG ( {2}^{} ; , ) Unif ( ℓ ; {a}_{ℓ} , {b}_{ℓ} ) . \qquad 7$$

The posterior distribution is proportional to the product of the likelihood function and prior density functions, however the evidence (the normalizing constant) is intractable. Thus, it is not feasible to directly use the joint posterior distribution for inference since the posterior distribution does not commit to a standard distribution family. Instead, we implemented the Markov Chain Monte Carlo (MCMC)⁷ method to draw a set of posterior samples of size S,

$$\{ ( {f}^{( s )} ( {t}_{1} ) , … , {f}^{( s )} ( {t}_{n} ) , {σ}^{{2}^{( s )}} , {{2}^{( s )}}^{} , {ℓ}^{( s )} ) , s = 1 , … , S \} .$$

We used the posterior sample mean as the estimate of the binding counts, as

$$\hat{f} ( {t}_{i} ) = \frac{1}{S} {\sum }_{s = 1}^{S} {f}^{( s )} ( {t}_{i} ) , i = 1 , … , n . \qquad 8$$

The posterior standard deviation in estimating \hat{ƒ}(tᵢ) is estimated by the standard deviation of the posterior samples

$${\hat{s}}_{i} = \sqrt{\frac{1}{S - 1} {\sum }_{s = 1}^{S} {\{ {f}^{( s )} ( {t}_{i} ) - \hat{f} ( {t}_{i} ) \}}^{2}} , i = 1 , … , n . \qquad 9$$

Equation 8 and 9 are used to provide posterior estimates of binding counts with S=1000 posterior samples when analyzing the real data.

Posterior computation. Markov Chain Monte Carlo (MCMC) algorithm generates Markov Chain samples iteratively, of which the distribution converges to the target (posterior) distribution as the number of iterations goes to infinity. In practice, it is justified that the distribution of the MCMC samples approximates the posterior distributions, we consider using the Gibbs sampling technique which is one of the common MCM algorithms used for Bayesian inference⁸. The Gibbs sampler iteratively samples the unknown parameters separately from their conditional posterior distributions, forming an updating circle: 1) sample from [ƒₙ|σ², [symbol] , [symbol] ]; 2) sample from [ [symbol] |ƒₙ, σ², [symbol] ]; 3) samples from [σ²|ƒₙ, [symbol] , [symbol] ] and 4) sample from [ [symbol] |σ², [symbol] , ƒₙ]. It is verified that the joint distribution of samples drawn from the updating circle also converges to the true posterior distribution. The detailed conditional distributions and corresponding sampling procedures are laid out as follows
1. Update [ƒₙ|σ², [symbol] , [symbol] , Y, T]˜N(μ_{ƒ}, Σ_{ƒ}) where

$${\sum }_{f} = {( \frac{{𝒯}_{ℓ}^{- 1}}{{2}^{}} + {σ}^{- 2} {I}_{n} )}^{- 1} and {μ}_{f} = \frac{{\sum }_{f} Y}{{σ}^{2}} ;$$

2. Update [ [symbol] |ƒₙ, σ², [symbol] , Y, T]˜IG(ãₜ, \tilde{b}ₜ) where ãₜ=aₜ+n/2 and

$$= + \frac{{f}_{n}^{T} {𝒯}_{ℓ}^{- 1} {f}_{n}}{2} .$$

We denote by ƒₙ^{T} the transpose of vector ƒₙ.
3. Update [σ²|ƒₙ, [symbol] , [symbol] , Y, T]˜IG(ã_{σ}, \tilde{b}_{σ}) where ã_{σ}=a_{σ}+n/2 and

$${\tilde{b}}_{σ} = {a}_{σ} + \frac{{( Y - {f}_{n} )}^{T} ( Y - {f}_{n} )}{2} .$$

4. Update

$$[ ℓ \vert {σ}^{2} , {2}^{} , f , Y , T ] ∝ {\vert {𝒯}_{ℓ} \vert}^{- 1 / 2} exp \{ \frac{{f}_{n}^{T} {𝒯}_{ℓ}^{- 1} {f}_{n}}{2} \} {𝕝}_{( {a}_{ℓ} , {b}_{ℓ} )} ( ℓ )$$

using Metropolis-Hasting algorithm. We write | [symbol] | as the determinant of matrix [symbol] , and [symbol] (x) denotes the indicator function of x in set A, that [symbol] (x)=1 if x∈A and [symbol] (x)=0 otherwise.

In fitting the real binding count data, we implemented 3000 iterations and discard the first 2000 iterations as burn-ins, and stored the last 1000 iterations as our posterior samples. Our empirical results also indicated that the Markov chain mixed well, the details are omitted here.

Some further aspects are also defined in the following clauses:
Clause 1: A method of detecting a target molecule, comprising: contacting a sample comprising the target molecule with a substrate that comprises a plurality of capture antibodies, or antigen binding portions thereof, that specifically bind to the target molecule to form captured target molecules, wherein the plurality of capture antibodies, or antigen binding portions thereof, are unlabeled; contacting the captured target molecules with a plurality of detection antibodies, or antigen binding portions thereof, that bind to the captured target molecules to form target molecule complexes, wherein the plurality of detection antibodies, or antigen binding portions thereof, are unlabeled; taking a series of dynamically tracked real-time images of the target molecule complexes over one or more selected periods of time to produce imaged target molecule complexes; and, quantifying an amount of target molecules in the sample using the imaged target molecule complexes, thereby detecting the target molecule.
Clause 2: The method of Clause 1, wherein the detection antibodies, or antigen binding portions thereof, specifically bind to a first epitope of the target molecules, wherein the capture antibodies, or antigen binding portions thereof, specifically bind to a second epitope of the target molecules, and wherein the first and second epitopes differ from one another.
Clause 3: The method of Clause 1 or Clause 2, wherein the target molecule complexes each comprise a single bound target molecule.
Clause 4: The method of any one of Clauses 1-3, wherein the quantifying step comprises digitally counting the imaged target molecule complexes in the images to quantify the amount of target molecule in the sample.
Clause 5: The method of any one of Clauses 1-4, wherein the quantifying step comprises determining a concentration of the target molecule in the sample.
Clause 6: The method of any one of Clauses 1-5, wherein the target molecule is a compound selected from the group consisting of: an interleukin-6 (IL-6) molecule, a prostate-specific antigen (PSA) molecules, and a N-terminal pro b-type natriuretic peptide (NT-proBNP) molecule.
Clause 7: The method of any one of Clauses 1-6, wherein the sample comprises buffer, serum, and/or whole blood.
Clause 8: The method of any one of Clauses 1-7, comprising flowing the captured target molecules through a plasma separator prior to contacting the captured target molecules with the plurality of detection antibodies, or antigen binding portions thereof.
Clause 9: The method of any one of Clauses 1-8, wherein the plurality of capture antibodies, or antigen binding portions thereof, are disposed on a surface of a solid support.
Clause 10: The method of any one of Clauses 1-9, comprising performing at least a portion of the method in a microfluidic device or system.
Clause 11: The method of any one of Clauses 1-10, comprising obtaining the sample from a subject.
Clause 12: The method of any one of Clauses 1-11, comprising administering, or discontinuing administering, therapy to the subject based at least in part on the amount of target molecule in the sample obtained from the subject.
Clause 13: The method of any one of Clauses 1-12, comprising generating a therapy recommendation for the subject based at least in part on the amount of target molecule in the sample obtained from the subject.
Clause 14: The method of any one of Clauses 1-13, wherein the quantifying step comprises filtering in terms of position, molecular weight, binding duration, and/or binding frequency of detected binding events in the imaged target molecule complexes, and determining and fitting a time course of a total count of specific binding of the detection antibodies, or antigen binding portions thereof, using a Gaussian Bayes algorithm.
Clause 15: A microfluidic device, comprising: a body structure comprising at least one microfluidic channel disposed at least partially in the body structure; a sample inlet area disposed at least partially in the body structure and in fluid communication with the microfluidic channel, wherein the sample inlet area is configured to receive sample aliquots that comprise mixtures of substantially unprocessed target molecules and a plurality of detection antibodies, or antigen binding portions thereof, that specifically bind to the target molecules in the sample; an assay area disposed at least partially in the body structure and in fluid communication with the microfluidic channel; a plurality of capture antibodies, or antigen binding portions thereof, disposed on a surface of the assay area, wherein the capture antibodies, or antigen binding portions thereof, specifically bind to the target molecules when the target molecules are conveyed from the sample inlet area to the assay area through at least a portion of the microfluidic channel into contact with the plurality of capture antibodies, or antigen binding portions thereof, to form captured target molecules; and, wherein the detection antibodies, or antigen binding portions thereof, specifically bind to the target molecules in the captured target molecules when the detection antibodies, or antigen binding portions thereof, are conveyed from the sample inlet area to the assay area through at least a portion of the microfluidic channel into contact with the captured target molecules to form target molecule complexes; wherein the microfluidic device is configured to operably connect to a fluid conveyance mechanism that effects fluid conveyance through the microfluidic channel to and/or from the sample inlet area and the assay area; and wherein the microfluidic device is configured to operably interface with a detection mechanism that images the target molecule complexes in the assay area to produce imaged target molecule complexes such that a controller operably connected to the detection mechanism quantifies an amount of target molecule in the sample aliquots from the imaged target molecule complexes.
Clause 16: The microfluidic device of Clause 15, wherein the detection antibodies, or antigen binding portions thereof, specifically bind to a first epitope of the target molecules, wherein the capture antibodies, or antigen binding portions thereof, specifically bind to a second epitope of the target molecules, and wherein the first and second epitopes differ from one another.
Clause 17: The microfluidic device of Clause 15 or Clause 16, wherein the target molecule complexes each comprise a single bound target molecule.
Clause 18: The microfluidic device of any one of Clauses 15-17, wherein the controller comprises a processor, and a memory communicatively coupled to the processor, the memory storing non-transitory computer executable instructions which, when executed on the processor, perform operations comprising: digitally counting the imaged target molecule complexes in the images to quantify the amount of target molecule in the sample aliquots.
Clause 19: The microfluidic device of any one of Clauses 15-18, wherein the amount of target molecule in the sample aliquots comprises a concentration of the target molecule in the sample aliquots.
Clause 20: The microfluidic device of any one of Clauses 15-19, wherein the detection mechanism comprises a bright-field microscope.
Clause 21: A kit comprising the microfluidic device of any one of Clauses 15-20.
Clause 22: The microfluidic device of any one of Clauses 15-21, wherein the sample inlet area comprises a sample inlet port.
Clause 23: The microfluidic device of any one of Clauses 15-22, wherein a microfluidic chip or cartridge comprises the microfluidic device.
Clause 24: The microfluidic device of any one of Clauses 15-23, wherein a point-of-care device or system comprises or is configured to receive the microfluidic device.
Clause 25: The microfluidic device of any one of Clauses 15-24, wherein the target molecule is a compound selected from the group consisting of: an interleukin-6 (IL-6) molecule, a prostate-specific antigen (PSA) molecules, and a N-terminal pro b-type natriuretic peptide (NT-proBNP) molecule.
Clause 26: The microfluidic device of any one of Clauses 15-25, wherein the sample comprises buffer, serum, and/or whole blood.
Clause 27: A system comprising the microfluidic device of any one of Clauses 15-26.
Clause 28: A computer readable media comprising non-transitory computer executable instruction which, when executed by at least electronic processor, perform at least: taking a rolling average of a raw image sequence to remove background noise to produce denoised images; converting the denoised images to probability images; identifying candidate pixels of a binding event in one or more frames of the probability images to produce identified candidate pixels; fitting a Gaussian function pixels of the denoised images with the same coordinates as the identified candidate pixels to produce a Gaussian fitting; filtering out invalid binding events and determining positions and intensities of valid binding events based on the Gaussian fitting to produce detected binding events; and, mapping the detected binding events in all frames of the denoised images over time with respect to spatial locations of the detected binding events.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of detecting a target molecule, comprising:
contacting a sample comprising the target molecule with a substrate that comprises a plurality of capture antibodies, or antigen binding portions thereof, that specifically bind to the target molecule to form captured target molecules, wherein the plurality of capture antibodies, or antigen binding portions thereof, are unlabeled;
contacting the captured target molecules with a plurality of detection antibodies, or antigen binding portions thereof, that bind to the captured target molecules to form target molecule complexes, wherein the plurality of detection antibodies, or antigen binding portions thereof, are unlabeled;
taking a series of dynamically tracked real-time images of the target molecule complexes over one or more selected periods of time to produce imaged target molecule complexes; and,
quantifying an amount of target molecules in the sample using the imaged target molecule complexes, thereby detecting the target molecule.

2. The method of claim 1, wherein the detection antibodies, or antigen binding portions thereof, specifically bind to a first epitope of the target molecules, wherein the capture antibodies, or antigen binding portions thereof, specifically bind to a second epitope of the target molecules, and wherein the first and second epitopes differ from one another.

3. The method of claim 1, wherein the quantifying step comprises filtering in terms of position, molecular weight, binding duration, and/or binding frequency of detected binding events in the imaged target molecule complexes, and determining and fitting a time course of a total count of specific binding of the detection antibodies, or antigen binding portions thereof, using a Gaussian Bayes algorithm.

4. The method of claim 1, wherein the quantifying step comprises digitally counting the imaged target molecule complexes in the images to quantify the amount of target molecule in the sample.

5. The method of claim 1, wherein the quantifying step comprises determining a concentration of the target molecule in the sample.

6. The method of claim 1, wherein the target molecule is a compound selected from the group consisting of: an interleukin-6 (IL-6) molecule, a prostate-specific antigen (PSA) molecules, and a N-terminal pro b-type natriuretic peptide (NT-proBNP) molecule.

7. The method of claim 1, wherein the sample comprises buffer, serum, and/or whole blood.

8. The method of claim 1, comprising flowing the captured target molecules through a plasma separator prior to contacting the captured target molecules with the plurality of detection antibodies, or antigen binding portions thereof.

9. The method of claim 1, wherein the plurality of capture antibodies, or antigen binding portions thereof, are disposed on a surface of a solid support.

10. The method of claim 1, comprising performing at least a portion of the method in a microfluidic device or system.

11. The method of claim 1, comprising obtaining the sample from a subject.

12. The method of claim 11, comprising administering, or discontinuing administering, therapy to the subject based at least in part on the amount of target molecule in the sample obtained from the subject.

13. The method of claim 11, comprising generating a therapy recommendation for the subject based at least in part on the amount of target molecule in the sample obtained from the subject.

14. A microfluidic device, comprising:
a body structure comprising at least one microfluidic channel disposed at least partially in the body structure;
a sample inlet area disposed at least partially in the body structure and in fluid communication with the microfluidic channel, wherein the sample inlet area is configured to receive sample aliquots that comprise mixtures of substantially unprocessed target molecules and a plurality of detection antibodies, or antigen binding portions thereof, that specifically bind to the target molecules in the sample;
an assay area disposed at least partially in the body structure and in fluid communication with the microfluidic channel;
a plurality of capture antibodies, or antigen binding portions thereof, disposed on a surface of the assay area, wherein the capture antibodies, or antigen binding portions thereof, specifically bind to the target molecules when the target molecules are conveyed from the sample inlet area to the assay area through at least a portion of the microfluidic channel into contact with the plurality of capture antibodies, or antigen binding portions thereof, to form captured target molecules; and,
wherein the detection antibodies, or antigen binding portions thereof, specifically bind to the target molecules in the captured target molecules when the detection antibodies, or antigen binding portions thereof, are conveyed from the sample inlet area to the assay area through at least a portion of the microfluidic channel into contact with the captured target molecules to form target molecule complexes;
wherein the microfluidic device is configured to operably connect to a fluid conveyance mechanism that effects fluid conveyance through the microfluidic channel to and/or from the sample inlet area and the assay area; and
wherein the microfluidic device is configured to operably interface with a detection mechanism that images the target molecule complexes in the assay area to produce imaged target molecule complexes such that a controller operably connected to the detection mechanism quantifies an amount of target molecule in the sample aliquots from the imaged target molecule complexes.

15. The microfluidic device of claim 14, wherein the detection antibodies, or antigen binding portions thereof, specifically bind to a first epitope of the target molecules, wherein the capture antibodies, or antigen binding portions thereof, specifically bind to a second epitope of the target molecules, and wherein the first and second epitopes differ from one another.

16. The microfluidic device of claim 14, wherein the controller comprises a processor, and a memory communicatively coupled to the processor, the memory storing non-transitory computer executable instructions which, when executed on the processor, perform operations comprising: digitally counting the imaged target molecule complexes in the images to quantify the amount of target molecule in the sample aliquots.

17. The microfluidic device of claim 14, wherein the detection mechanism comprises a bright-field microscope.

18. A kit comprising the microfluidic device of claim 14.

19. The microfluidic device of claim 14, wherein the target molecule is a compound selected from the group consisting of: an interleukin-6 (IL-6) molecule, a prostate-specific antigen (PSA) molecules, and a N-terminal pro b-type natriuretic peptide (NT-proBNP) molecule.

20. A computer readable media comprising non-transitory computer executable instruction which, when executed by at least electronic processor, perform at least:
taking a rolling average of a raw image sequence to remove background noise to produce denoised images;
converting the denoised images to probability images;
identifying candidate pixels of a binding event in one or more frames of the probability images to produce identified candidate pixels;
fitting a Gaussian function pixels of the denoised images with the same coordinates as the identified candidate pixels to produce a Gaussian fitting;
filtering out invalid binding events and determining positions and intensities of valid binding events based on the Gaussian fitting to produce detected binding events; and,
mapping the detected binding events in all frames of the denoised images over time with respect to spatial locations of the detected binding events.
